SEQUENCE LISTING (2 sequences)

SEQ ID NO: 1 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0304688 A1
(43) Pub. Date: Oct. 2, 2025

(54) Methods and Compositions Comprising Anti-CD3 Antibodies and DYRK1A Inhibitors for Treating Diabetes

(71) Applicant: Provention Bio, Inc., Red Bank, NJ (US)

(72) Inventor: Francisco LEON, Bethesda, MD (US)

(73) Assignee: Provention Bio, Inc., Red Bank, NJ (US)

(21) Appl. No.: 18/691,228

(22) PCT Filed: Sep. 13, 2022
(86) PCT No.: PCT/US2022/043383

§ 371 Date: Mar. 12, 2024

Related U.S. Application Data
(60) Provisional application No. 63/243,666, filed on Sep. 13, 2021.
(60) Provisional application No. 63/318,363, filed on Mar. 9, 2022.

(51) Int. Cl. C07K 16/28; A61K 39/00; A61K 45/06

(52) U.S. Cl. CPC C07K 16/2809; A61K 45/06; A61K 2039/545; C07K 2317/24

(57) ABSTRACT

Provided herein are methods and compositions for treating type 1 diabetes. Also provided herein are methods and compositions for preventing or delaying the onset of type 1 diabetes. In some embodiments, such method can include administering to a subject in need thereof a 12-day to 14-day course of teplizumab at a total dose of about 9000 μg/m² to about 14000 μg/m² and administering an effective amount of a DYRK1A inhibitor.

### RELATED APPLICATIONS

This application is a national stage application under 35 U.S.C. § 371 of International Patent Application No. PCT/US2022/043383, filed Sep. 13, 2022, which claims priority to and the benefit of U.S. Provisional Application No. 63/243,666, filed Sep. 13, 2021 and U.S. Provisional Application No. 63/318,363, filed Mar. 9, 2022, the entire disclosures of each of which are incorporated herein by reference.

### SEQUENCE LISTING

This specification includes a sequence listing submitted electronically herewith that was created on Nov. 18, 2024, is entitled 122548.US056.xml, and has the size of 3,464 bytes. The contents of the sequence listing are incorporated by reference herein.

### FIELD

The present disclosure relates in general to methods and compositions for treating diabetes in subjects in need thereof.

### BACKGROUND

Type 1 diabetes (T1D) is caused by the autoimmune destruction of insulin producing beta cells in the islets of Langerhans leading to dependence on exogeneous insulin injections for survival. Approximately 1.6 million Americans have Type 1 diabetes, and after asthma, it remains one of the most common diseases of childhood. Despite improvements in care, most affected individuals with T1D are not able to consistently achieve desired glycemic targets. For individuals with type 1 diabetes, there are persisting concerns for increased risk of both morbidity and mortality. Two recent studies noted loss of 17.7 life-years for children diagnosed before age 10, and 11 and 13 life-years lost for adult-diagnosed Scottish men and women respectively.

Thus, a need exists for improved T1D treatment methods and compositions.

### SUMMARY

One aspect of the disclosure relates to a method of treating type 1 diabetes (T1D), comprising: administering to a subject in need thereof a 12-day to 14-day course of teplizumab at a total dose of about 9000 μg/m² to about 14000 μg/m², and administering to the subject an effective amount of a DYRK1A inhibitor.

In some embodiments, the method comprises administering to the subject in need thereof a 12-day course, wherein the 12-day course comprises a first dose of 106 μg/m² teplizumab on day 1, a second dose of 425 μg/m² teplizumab on day 2, and one dose of 850 μg/m² on each of days 3-12, and wherein the total dose is approximately 9031 μg/m².

In some embodiments, the method comprises administering to the subject in need thereof a 12-day course, wherein the 12-day course comprises a first dose of 211 μg/m² teplizumab on day 1, a second dose of 423 μg/m² teplizumab on day 2, and one dose of 840 μg/m² on each of days 3-12, and wherein the total dose is approximately 9034 μg/m².

In some embodiments, the method includes administering a first and a second 12-day courses of teplizumab. In some embodiments, the first and the second 12-day courses are administered at about 6 months interval. In some embodiments, the first and the second 12-day courses are administered at about 1-6 months, about 2-5 months or about 3 months interval.

In some embodiments, the method includes administering to the subject in need thereof a third or more 12-day course of teplizumab, each course at a total dose of about 9000 μg/m² to about 14000 μg/m².

In some embodiments, the third or more 12-day course of teplizumab comprises a first dose of 106 μg/m² teplizumab on day 1, a second dose of 425 μg/m² teplizumab on day 2, and one dose of 850 μg/m² on each of days 3-12, and wherein the total dose of each course is approximately 9031 μg/m².

In some embodiments, the third or more 12-day course of teplizumab comprises a first dose of 211 μg/m² teplizumab on day 1, a second dose of 423 μg/m² teplizumab on day 2, and one dose of 840 μg/m² on each of days 3-12, and wherein the total dose of each course is approximately 9034 μg/m².

In some embodiments, the third or more 12-day course of teplizumab is administered to the subject in need thereof at about a 12 month to about a 24-month interval.

In some embodiments, the method further includes determining, at baseline and about 3 months after the administration, the level of TIGIT+KLRG1+CD8+ cells with respect to all CD3+ T cells; monitoring the level of the TIGIT+KLRG1+CD8+CD3+ T-cells; and administering an additional 12-day course of teplizumab when the level of the TIGIT+KLRG1+CD8+CD3+ T-cells returns to the baseline level. In some embodiments, the determining of TIGIT+KLRG1+CD8+CD3+ T-cells is by flow cytometry. In some embodiments, the monitoring of TIGIT+KLRG1+CD8+CD3+ T-cells is by flow cytometry. In some embodiments, if the subject has more than about 10% TIGIT+KLRG1+CD8+ T-cells in all CD3+ T cells, subsequent monitoring is annual. In some embodiments, if the subject has less than about 10% TIGIT+KLRG1+CD8+ T-cells in all CD8+ T cells, subsequent monitoring is every about 3-6 months or every about 6 months.

In some embodiments, the administrating step results in reduction by at least 10% of exogeneous insulin use, HbA1c levels, hypoglycemic episodes, or combinations thereof as compared to pre-treatment levels.

In some embodiments, each dose of teplizumab is administered parenterally.

In some embodiments, each dose of teplizumab is administered by intravenous infusion.

In some embodiments, each dose of teplizumab is administered by subcutaneously.

In some embodiments, each dose of teplizumab is administered by orally.

In some embodiments, the effective amount of DYRK1A inhibitor is administered orally, intraperitoneally, subcutaneously or by intravenous infusion.

In some embodiments, the teplizumab and DYRK1A inhibitor are co-administered.

In some embodiments, the subject in need thereof is about 8 to 17 years old.

In some embodiments, the subject in need thereof has a peak C-peptide level of ≥0.2 pmol/mL during a mixed meal tolerance test (MMTT).

In some embodiments, the subject receiving teplizumab has a higher mean C-peptide value after treatment, compared with a control receiving placebo.

In some embodiments, the method further includes assessing the area under the time-concentration curve (AUC) of C-peptide following a mixed meal tolerance test (MMTT), at 78 weeks.

In some embodiments, the subject in need thereof has at least 20% of beta-cell function prior the administration of the teplizumab and the DYRK1A inhibitor.

In some embodiments, the reduction of exogeneous insulin use, HbA1c levels, hypoglycemic episodes, or combinations thereof is over a period of 12 months or more.

In some embodiments, the method comprises administering a 14 day course at about 60 μg/m² on day 1, about 125 μg/m² on day 2, about 250 μg/m² on day 3, and about 500 μg/m² on day 4, and one dose of about 1000 μg/m² on each of days 5-14. In some embodiments, the subject in need thereof is a non-diabetic subject who is at risk for TID.

In some embodiments, the method comprises administering a 14 day course at about 60 μg/m² on day 1, about 125 μg/m² on day 2, about 250 μg/m² on day 3 and about 500 μg/m² on day 4, respectively, and one dose of about 1030 μg/m² on each of days 5-14. In some embodiments, the subject in need thereof is a non-diabetic subject who is at risk for TID.

In some embodiments, the method comprises administering a 14 day course at about 100 μg/m² on day 1, about 425 μg/m² on day 2, about 850 μg/m² on day 3, and about 850 μg/m² on day 4, and one dose of about 1000 μg/m² on each of days 5-14. In some embodiments, the subject in need thereof is a non-diabetic subject who is at risk for T1D.

In some embodiments, the method comprises administering a 14 day course at about 65 μg/m² on day 1, about 125 μg/m² on day 2, about 250 μg/m² on day 3, and about 500 μg/m² on day 4, and one dose of about 1070 μg/m² on each of days 5-14. In some embodiments, the subject in need thereof is a non-diabetic subject who is at risk for T1D.

In some embodiments, each dose of teplizumab is administered parenterally. In some embodiments, each dose of teplizumab is administered by intravenous infusion. In some embodiments, each dose of teplizumab is administered subcutaneously. In some embodiments, each dose of teplizumab is administered orally.

Aspects of the disclosure relate to methods of preventing or delaying onset of type 1 diabetes (T1D), comprising: administering prophylactically to a subject in need thereof a 14-day course of teplizumab at a total dose of about 9000 μg/m² to about 14000 μg/m² and administering to the subject in need thereof an effective amount of a DYRK1A inhibitor.

In some embodiments, the prophylactically effective amount of teplizumab is administered subcutaneously (SC) or intravenously (IV) or orally. [0035]39 In some embodiments, the method comprises administering a 14 day course IV infusion at about 60 μg/m² on day 1, about 125 μg/m² on day 2, about 250 μg/m² on day 3, and about 500 μg/m² on day 4, and one dose of about 1000 μg/m² on each of days 5-14.

In some embodiments, the method comprises administering a 14 day course IV infusion at about 60 μg/m² on day 1, about 125 μg/m² on day 2, about 250 μg/m² on day 3 and about 500 μg/m² on day 4, respectively, and one dose of about 1030 μg/m² on each of days 5-14.

In some embodiments, the method comprises administering a 14 day course IV infusion at about 100 μg/m² on day 1, about 425 μg/m² on day 2, about 850 μg/m² on day 3, and about 850 μg/m² on day 4, and one dose of about 1000 μg/m² on each of days 5-14.

In some embodiments, the method comprises administering a 14 day course IV infusion at about 65 μg/m² on day 1, about 125 μg/m² on day 2, about 250 μg/m² on day 3, and about 500 μg/m² on day 4, and one dose of about 1070 μg/m² on each of days 5-14.

Aspects of the disclosure relate to methods of treating type 1 diabetes (T1D), the method comprising: administering intravenously to a subject in need thereof a 12-day course of teplizumab at a total dose of about 9000 μg/m² to about 14000 μg/m²; and administering to the subject in need thereof an effective amount of a DYRK1A inhibitor.

Aspects of the disclosure relate to methods of treating type 1 diabetes (T1D), the method comprising administering subcutaneously to a subject in need thereof a 12-day course of teplizumab at a total dose of about 9000 μg/m² to about 14000 μg/m²; and administering to the subject in need thereof an effective amount of a DYRK1A inhibitor.

Aspects of the disclosure relate to a combination of teplizumab and DYRK1A inhibitor for treating type 1 diabetes comprising: administering subcutaneously to a subject in need thereof a 12-day course of teplizumab at a total dose of about 9000 μg/m² to about 14000 μg/m²; and administering to the subject in need thereof an effective amount of a DYRK1A inhibitor.

Aspects of the disclosure relate to a combination of teplizumab and DYRK1A inhibitor for preventing or delaying onset of type 1 diabetes, comprising: administering a prophylactically to a subject in need thereof a 14-day course of teplizumab at a total dose of about 9000 μg/m² to about 14000 μg/m² and administering to the subject in need thereof an effective amount of a DYRK1A inhibitor.

### BRIEF DESCRIPTION OF THE DRAWINGS

FIG. 1: Simulated Concentrations for Three Dosing Regimens: Population Predictions for a Typical Male Patient with WT=60 kg, Age=18 years, BSA=1.67 m², and no Detected ADAs.

FIG. 2: Comparison of Concentrations for Dosing Regimens 1 and 2: Model-based Simulations for a Typical Male Patients with WT=60 kg, Age=18 years, BSA=1.67 m², and no Detected ADAs.

FIG. 3: Comparison of Concentrations for Herold Dosing Regimen and Dosing Regimen 1: Model-based Simulations for a Typical Male Patients with WT=60 kg, Age=18 years, BSA=1.67 m², and no Detected ADAs.

FIG. 4: Comparison of Concentrations on the last dosing day for Herold Dosing Regimen and Dosing Regimen 1: Model-based Simulations for a Typical Male Patients with WT=60 kg, Age=18 years, BSA=1.67 m², and no Detected ADAs.

FIG. 5: Simulated Concentrations For Three Dosing Regimens: Population Predictions for a Typical Male Patient with WT=60 kg, Age=18 years, BSA=1.67 m², and High Level of Detected ADAs.

FIG. 6: Comparison of Concentrations for Dosing Regimens 1 and 2: Model-based Simulations for Typical Male Patient with WT=60 kg, Age=18 years, BSA=1.67 m², and High Level of Detected ADAs.

FIG. 7: Comparison of Concentrations for Herold Dosing Regimen and Dosing Regimen 1: Model-based Simulations for Typical Male Patients with WT=60 kg, Age=18 years, BSA=1.67 m², and High Level of Detected ADAs.

FIG. 8: Comparison of Concentrations on the last dosing day for Herold Dosing Regimen and Dosing Regimen 1: Model-based Simulations for Typical Male Patients with WT=60 kg, Age=18 years, BSA=1.67 m², and High Level of Detected ADAs.

FIG. 9: Simulated Concentrations For Three Dosing Regimens: Population Predictions for a Typical Male Patient with WT=45 kg, Age=13 years, BSA=1.33 m², and no Detected ADAs.

FIG. 10: Comparison of Concentrations for Dosing Regimens 1 and 2: Model-based Simulations for Typical Male Patients with WT=45 kg, Age=13 years, BSA=1.33 m², and no Detected ADAs.

FIG. 11: Comparison of Concentrations for Herold Dosing Regimen and Dosing Regimen 1: Model-based Simulations for Typical Male Patients with WT=45 kg, Age=13 years, BSA=1.33 m², and no Detected ADAs.

FIG. 12: Comparison of Concentrations on the last dosing day for Herold Dosing Regimen and Dosing Regimen 1: Model-based Simulations for Typical Male Patients with WT=45 kg, Age=13 years, BSA=1.33 m², and no Detected ADAs.

FIG. 13: Simulated Concentrations For Three Dosing Regimens: Population Predictions for a Typical Male Patient with WT=45 kg, Age=13 years, BSA=1.33 m², and High Level of Detected ADAs.

FIG. 14: Comparison of Concentrations for Dosing Regimens 1 and 2: Model-based Simulations for Typical Male Patients with WT=45 kg, Age=13 years, BSA=1.33 m², and High Level of Detected ADAs.

FIG. 15: Comparison of Concentrations for Herold Dosing Regimen and Dosing Regimen 1: Model-based Simulations for Typical Male Patients with WT=45 kg, Age=13 years, BSA=1.33 m², and High Level of Detected ADAs.

FIG. 16: Comparison of Concentrations on the last dosing day for Herold Dosing Regimen and Dosing Regimen 1: Model-based Simulations for Typical Male Patients with WT=45 kg, Age=13 years, BSA=1.33 m², and High Level of Detected ADAs.

FIG. 17: Comparison of Concentrations for Herold Regimen and Dosing Regimen 2: Model-based Simulations for Male Patients with WT=60 kg, Age=18 years, BSA=1.67 m², and no Detected ADAs (42 days).

FIG. 18: Comparison of Median Concentrations for Herold Regimen and Dosing Regimen 2: Model-based Simulations for Male Patients with WT=60 kg, Age=18 years, BSA=1.67 m², and no Detected ADAs (35 days).

FIG. 19: Comparison of Concentrations for Herold Regimen and Dosing Regimen 2: Model-based Simulations for Male Patients with WT=60 kg, Age=18 years, BSA=1.67 m², and High Level of Detected ADAs (42 days)

FIG. 20: Comparison of Median Concentrations for Herold Regimen and Dosing Regimen 2: Model-based Simulations for Male Patients with WT=60 kg, Age=18 years, BSA=1.67 m², and High Level of Detected ADAs (35 days).

FIG. 21: Comparison of Concentrations for Herold Regimen and Dosing Regimen 2: Model-based Simulations for Male Patients with WT=45 kg, Age=13 years, BSA=1.33 m², and no Detected ADAs (42 days).

FIG. 22: Comparison of Median Concentrations for Herold Regimen and Dosing Regimen 2: Model-based Simulations for Male Patients with WT=45 kg, Age=13 years, BSA=1.33 m², and no Detected ADAs (35 days).

FIG. 23: Comparison of Concentrations for Herold Regimen and Dosing Regimen 2: Model-based Simulations for Male Patients with WT=45 kg, Age=13 years, BSA=1.33 m², and High Level of Detected ADAs (42 days).

FIG. 24: Comparison of Median Concentrations for Herold Regimen and Dosing Regimen 2: Model-based Simulations for Male Patients with WT=45 kg, Age=13 years, BSA=1.33 m², and High Level of Detected ADAs (35 days).

FIG. 25 shows a diagram of the study design according to one embodiment.

FIG. 26: Predicted Mean Difference Between Teplizumab and Control in the Change from Baseline in C-Peptide AUC (nmol/L) at 1 Year Follow-up in Supportive Study Meta-Analysis.

FIG. 27: Predicted Mean Difference Between Teplizumab and Control in the Change from Baseline in C-peptide AUC (nmol/L) at 2 Year Follow-up in Supportive Study Meta-Analysis.

FIG. 28: TN-10: C-Peptide AUCs (nmol/L) in Patients with T1D.

FIG. 29: Average Insulin Use at Each Visit.

FIG. 30: Predicted Mean Teplizumab Serum Concentration Versus Time Profile Following 14-Day Regimen Across Different Body Weights.

### DETAILED DESCRIPTION

Type 1 diabetes usually develops in childhood and adolescence; however, it can also present in adulthood as late as the 5th and 6th decades of life, although much less frequently (Atkinson 2014, Bluestone 2010, Streisand 2014). In addition to being more prone to some short- and long-term complications, there are differences in the clinical course and response to immune therapies between children/young adults and older adults. In the days or weeks before initial diagnosis, children and adolescents often suffer from severe diabetes symptoms, including polydipsia, polyuria, and weight loss, which could result in a clinical presentation of DKA and shock which requires hospitalization (Atkinson 2014, Bluestone 2010, Streisand 2014, Mittermayer 2017). Children and young adults with new-onset T1D usually have an immediate need for exogenous insulin.

This sharply contrasts with the experience of adults who develop TiD who often have months or years of non-specific symptoms or present asymptomatically from routine glycemic screening. These individuals can often be managed for prolonged periods of time (months or years) with diet or oral hypoglycemic agents before a demonstrable exogeneous insulin need. More definitive studies have shown a different rate of decline of β cells according to age (Greenbaum 2012; Ludvigsson 2013). Following decades of study, the Diabetes TrialNet network has concluded that “age is the most important factor impacting the rate of decline of C-peptide post diagnosis” in that a significantly more rapid rate of decline occurs in children and adolescents compared to younger and older adults with new-onset disease. This more rapid decline appears to be due to a much more virulent and aggressive autoimmune process in children compared to adults, ostensibly supporting that there are important differences in T1D immuno-pathoetiology in younger versus older individuals (Greenbaum 2012, Campbell-Thompson 2016). Due to these fundamental differences, it is reasonable to expect that adults and children may respond differently to an immune-based disease modifying therapy. In other words, one treatment may be very effective in children but not effective at all in adults and vice versa (Rigby 2014).

Children and adolescents are those at highest risk of developing disease and suffer most substantially from short- and long-term morbidity and mortality, and thus this group has the most to benefit from a disease modifying therapy (Wherrett 2015). This has recently been reinforced by a large study showing that those diagnosed with T1D in childhood and adolescence have a 4-6-fold increase in lifetime mortality risk, including seven times the risk of mortality from cardiovascular disease, compared to counterparts without T1D. This mortality risk is in sharp contrast to individuals diagnosed with T1D in adulthood, who have a ˜3-fold higher risk from all-cause and cardiovascular disease-related mortality compared to their otherwise healthy peers (Rawshani 2017, Rawshani 2018). Recent reports indicate that those with T1D have a life expectancy ˜11-13 years less than otherwise healthy-age matched individuals (Lind 2014, Huo 2016). While it is a goal in T1D research to reduce the morbidity and mortality for all with T1D, it is apparent that the most urgent need is for those who develop T1D in childhood and adolescence.

There is therefore a need to develop a therapy for children who can most likely benefit from it. Such therapy benefits adolescents and adults with T1D as well.

T1D is characterized by destruction of most insulin-producing beta cells by an autoimmune response. Patients with established T1D have residual beta cells, but these do not thrive due to the autoimmune disease, which destroys them upon proliferation. There are 4 stages of T1D: stage 1—multiple (at least 2) islet antibodies, normal blood glucose, pre-symptomatic; stage 2—multiple islet antibodies, raised blood glucose, pre-symptomatic; stage 3—islet autoimmunity, raised blood glucose, symptomatic; stage 4—long standing type 1 diabetes. In some aspects of the disclosure, the method results in regeneration of beta cells.

High-throughput screening has been used to identify small molecules able to stimulate beta cell regeneration and expansion (Aamodt et al., Am J Physiol Endocrinol Metab. 2016 Nov. 1; 311(5):E859-E868). It has been found that harmine-mediated inhibition of DYRK1A increased pancreatic beta cell replication (Wang et al, Nat Med. 2015 April; 21(4):383-8; Kumar et al., J Med Chem. 2018 Sep. 13; 61(17):7687-7699). These inhibitors are synergistic with GLP-1 receptor agonists, which may additionally be cross-linked to the DYRK1A inhibitors to increase their pancreatic tropism (Ackeifi et al., JCI Insight. 2020 Jan. 16; 5(1):e132594). Aspects of the disclosure relate to methods of treating type 1 diabetes (T1D) in subjects in need thereof. Provided herein are methods that preserve β cell function, regenerate β cells/increase pancreatic β cells proliferation and/or improve clinical management of T1D in subjects in need thereof compared with the natural course of disease and current standard of care including exogenous insulin therapy. The preservation and/or amelioration of β cell function is anticipated to translate to clinical and/or metabolic benefits consistent with improved ability to maintain glycemic control and short- and/or long-term outcomes.

Aspects of the disclosure relate to combination therapy for the treatment of T1D. In some embodiments, the methods of treating type 1 diabetes (T1D) comprises administering to the subject in need thereof an effective amount of one or more anti-CD3 antibody in combination with an effective amount of one or more DYRK1A inhibitor. DYRK1A inhibitors induce proliferation in human beta cells via their ability to induce nuclear translocation of nuclear factor of activated T-cells (“NFaTs”), transcription factors that then trans-activate cell cycle-activating genes and repress cell cycle inhibitor genes.

In some embodiments, the method comprises administering to the subject in need thereof a combination therapy comprising a course of one or more anti-CD3 antibody and a course of one or more DYRK1A inhibitor to induce immune tolerance to the regenerated beta cells, in patients with T1D. In some embodiments, the one or more anti-CD3 antibody comprises or consists of teplizumab. In some embodiments, the anti-CD3 antibody can be ChAglyCD3 (otelixizumab), visilizumab or foralumab.

In some embodiments, the course of anti-CD3 antibody is a 8- to 16-day course. In some embodiments, the course of anti-CD3 antibody is a 12-day course (see U.S. application No. 63/192,402, filed May 24, 2021, which is incorporated herein by reference in its entirety). In some embodiments, the method comprises administering to a subject in need thereof a first course of daily doses of teplizumab for 12 days, and a second course of daily doses of teplizumab for 12 days, wherein the first and second courses are separated with a 6-month interval.

In some embodiments, the anti-CD3 antibody is teplizumab.

In some embodiments, the method comprises administering to the subject in need thereof a combination therapy comprising a 12-day anti-CD3 antibody course in combination with a course of DYRK1A inhibitor to induce immune tolerance to the regenerated beta cells, in patients with T1D. In some embodiments, the anti-CD3 antibody comprises or consists of teplizumab. In some embodiments, the anti-CD3 agent is not teplizumab. In some embodiments, the anti-CD3 antibody can be ChAglyCD3 (otelixizumab), visilizumab or foralumab.

In some embodiments, the method comprises diagnosing patients with T1D. In some embodiments, the patients are in Stage 2 (pre-symptomatic), Stage 3 (newly diagnosed) or Stage 4 (established disease).

In some embodiments, patients have other variants of autoimmune diabetes: LADA, type 2 diabetes with autoimmune phenomena (evidenced by at least 1 T1D AA), Stage 1 with a single high-affinity AA, post-viral diabetes, post-checkpoint inhibitor diabetes, or post-gestational diabetes T1D.

In some embodiments, the method comprises determining presence of residual beta cell mass: by (1) oral-glucose-tolerance test (OGTT, 2 or 4 hours test) prior to treatment, (2) demonstration of detectable C-peptide prior to treatment, or combination thereof. In some embodiments, the subject in need thereof has at least 20% of beta-cell function prior the administration of the combination therapy.

In some embodiments, the method comprises administering to the subject in need thereof within 6 weeks of diagnosis a first course of daily doses of teplizumab for 12 days, and a second course of daily doses of teplizumab for 12 days, wherein the first and second courses are separated with a 6-month interval.

In some embodiments, the method further comprises assessing the area under the time-concentration curve (AUC) of C-peptide following a mixed meal tolerance test (MMTT), at 78 weeks (18 months or 1.5 years), and/or evaluating clinical endpoints such exogenous insulin use, HbA1c levels, and hypoglycemic episodes.

In some embodiments, the method comprises diagnosing patients with T1D, administering to the patients (e.g., within 6 weeks of diagnosis) a first course of daily doses of teplizumab for 12 days, and a second course of daily doses of teplizumab for 12 days, wherein the first and second courses are separated with a 6-month interval. In some embodiments, the method further comprises assessing the area under the time-concentration curve (AUC) of C-peptide following a mixed meal tolerance test (MMTT), at 78 weeks (18 months or 1.5 years), and/or evaluating clinical endpoints such exogenous insulin use, HbA1c levels, and hypoglycemic episodes.

### Definitions

Certain terms are defined herein below. Additional definitions are provided throughout the application.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one”.

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

As used herein, the term “onset” of disease with reference to Type-1 diabetes refers to a patient meeting the criteria established for diagnosis of Type-1 diabetes by the American Diabetes Association (see, Mayfield et al., 1998, Am. Fam. Physician 58:1355-1362).

As used herein, a “protocol” includes dosing schedules and dosing regimens. The protocols herein are methods of use and include therapeutic protocols. A “dosing regimen”, “dosage regimen” or “course of treatment” may include administration of several doses of a therapeutic agent over 1 to 20 days.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey or a human), and more preferably a human. In some embodiments, the patient population comprises children. In some embodiments, the patient population comprises children newly diagnosed with T1D. In some embodiments, the patient population is treated within 6 weeks of the T1D diagnosis. In some embodiments, the patient population comprises children who are positive for at least one T1D-associated autoantibody and have a peak stimulated C-peptide of ≥0.2 pmol/mL at screening.

As used herein, the term “children” (and variations thereof) includes those being around 8 to 17 years of age.

As used herein, the terms “effective amount” and “therapeutically effective amount” are used interchangeably and refer to that amount of active agents sufficient to result in the treatment of T1D. For example, an effective amount of teplizumab is the amount of teplizumab sufficient to result in the delay or prevention of the development, recurrence or onset of one or more symptoms of T1D. An effective amount of DYRK1A inhibitors is the mount sufficient to result in the regeneration of beta cells.

As used herein, the terms “treat”, “treatment” and “treating” refer to the amelioration of one or more symptoms associated with TiD that results from the administration of one or more CD3 binding molecules and one or more DYRK1A inhibitors. In some embodiments, such terms refer to a reduction in a human's average number of hypoglycemic episodes. In other embodiments, such terms refer to the maintenance of a reference level of C-peptide in the peripheral blood.

In some embodiments, the effective amount reduces one or more T1D symptoms by at least 5%, by at least 10%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 96%, by at least 97%, by at least 98% or by at least 99%.

As used herein, the term “beta-cell regeneration” refers to at least a partial restoration of normal beta-cell function by increasing the number of functional insulin secreting beta-cells and/or by restoring normal function in functionally impaired beta-cells.

The terms “synergy” and “synergistic” as used herein, means that the effect achieved with the compounds used together is greater than the sum of the effects that results from using the compounds separately, i.e. greater than what would be predicted based on the two active ingredients administered separately.

As used herein, the term “prophylactic agent” refer to CD3 binding molecules such as teplizumab which can be used in the prevention, delay, treatment, management or amelioration of one or more symptoms of T1D.

As used herein, the term “prophylactically effective amount” refers to that amount of teplizumab sufficient to result in the delay or prevention of the development, recurrence or onset of one or more symptoms of T1D. In some embodiments, a prophylactically effective amount preferably refers to the amount of teplizumab that delays a subject's onset of T1D by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%.

As used herein, the terms “prevent”, “preventing” and “prevention” refer to the prevention or delay of the onset of one or more symptoms of T1D in a subject resulting from the administration of a prophylactic or therapeutic agent.

As used herein, the term “co-administration”, “co-administer”, “co-administered” is a reference to the administration of more than one active agent to treat a disease or condition in a patient. The term “co-administration” includes, but is not limited to, administration of two or more active agents simultaneously in a single dosage form (e.g. a fixed-dose combination tablet), administration of two or more active agents simultaneously in separate dosage forms, and administration of two or more active agents sequentially in separate dosage forms. When two or more active agents are co-administered sequentially, they are administered separately with a time interval between each administration.

Various aspects of the disclosure are described in further detail below. Additional definitions are set out throughout the specification.

### Anti-CD3 Antibodies and Pharmaceutical Compositions Comprising Anti-CD3 Antibodies

The terms “anti-CD3 antibody” and “an antibody that binds to CD3” refer to an antibody or antibody fragment that is capable of binding cluster of differentiation 3 (CD3) with sufficient affinity such that the antibody is useful as a prophylactic, diagnostic and/or therapeutic agent in targeting CD3. In some embodiments, the extent of binding of an anti-CD3 antibody to an unrelated, non-CD3 protein is less than about 10% of the binding of the antibody to CD3 as measured, e.g., by a radioimmunoassay (RIA). In some embodiments, an antibody that binds to CD3 has a dissociation constant (Kd) of <1 μM, <100 nM, <10 nM, <1 nM, <0.1 nM, <0.01 nM, or <0.001 nM (e.g. 10⁻⁸ M or less, e.g. from 10⁻⁸ M to 10⁻¹³ M, e.g., from 10⁻⁹ M to 10⁻¹³ M). In some embodiments, an anti-CD3 antibody binds to an epitope of CD3 that is conserved among CD3 from different species.

In some embodiments, the anti-CD3 antibody can be ChAglyCD3 (otelixizumab). Otelixizumab is a humanized Fc nonbinding anti-CD3, which was evaluated initially in phase 2 studies by the Belgian Diabetes Registry (BDR) and then developed by Tolerx, which then partnered with GSK to conduct the phase 3 DEFEND new onset T1D trials (NCT00678886, NCT01123083, NCT00763451). Otelixizumab is administered IV with infusions over 8 days. See, e.g., Wiczling et al., J. Clin. Pharmacol. 50 (5) (May 2010) 494-506; Keymeulen et al., N Engl J Med. 2005; 352:2598-608; Keymeulen et al., Diabetologia. 2010; 53:614-23; Hagopian et al., Diabetes. 2013; 62:3901-8; Aronson et al., Diabetes Care. 2014; 37:2746-54; Ambery et al., Diabet Med. 2014; 31:399-402; Bolt et al., Eur. J. Immunol. 1YY3. 23: 403-411; Vlasakakis et al., Br J Clin Pharmacol (2019) 85 704-714; Guglielmi et al, Expert Opinion on Biological Therapy, 16:6, 841-846; Keymeulen et al., N Engl J Med 2005; 352:2598-608; Keymeulen et al., Blood 2010, Vol. 115, No. 6; Sprangers et al., Immunotherapy (2011) 3(11), 1303-1316; Daifotis et al., Clinical Immunology (2013) 149, 268-278; all incorporated herein by reference.

In some embodiments, the anti-CD3 antibody can be visilizumab (also called HuM291; Nuvion). Visilizumab is a humanized anti-CD3 monoclonal antibody characterized by a mutated IgG2 isotype, lack of binding to Fcγ receptors, and the ability to induce apoptosis selectively in activated T cells. It was evaluated in patients in graft-versus-host disease (NCT00720629; NCT00032279) and in ulcerative colitis (NCT00267306) and Crohn's Disease (NCT00267709). See, e.g., Sandborn et al., Gut 59 (11) (November 2010) 1485-1492, incorporated herein by reference.

In some embodiments, the anti-CD3 antibody can be foralumab (also called TZLS-401). Foralumab is a fully human monoclonal antibody that binds to CD3 epsilon. (see U.S. Pat. No. 10,688,186 incorporated by reference in its entirety.)

### Teplizumab

In some embodiments, the anti-CD3 antibody can be teplizumab. Teplizumab, also known as hOKT3yl(Ala-Ala) (containing an alanine at positions 234 and 235) is an anti-CD3 antibody that had been engineered to alter the function of the T lymphocytes that mediate the destruction of the insulin-producing beta cells of the islets of the pancreas. Teplizumab binds to an epitope of the CD3E chain expressed on mature T cells and by doing so changes their function. Circulating T cells (and other lymphocytes) are transiently reduced following teplizumab treatment, in a process that may include margination and depletion (Long 2017, Sherry 2011). In addition to reduced effector function of T cells, teplizumab appears to both increase the number and function of regulatory T cells (Tregs) (Ablamunits 2010, Bisikirska 2005, Long 2017, Waldron-Lynch 2012). More recent studies indicate that teplizumab induces immunologic “exhaustion” in a subset of effector CD8+ T cells, perhaps making them more susceptible to regulation or deletion (Long 2016, Long 2017). Taken together, these mechanistic data suggest that teplizumab not only exerts a “suppressive” effect on β cell immune destructive processes but rather is an immune “modulator” favoring a rebalancing of effector and regulatory arms involved with T1D autoimmunity and supporting the notion that teplizumab may have the ability to contribute to the re-introduction of β cell self-tolerance (Lebastchi 2013).

Sequences and compositions of teplizumab are disclosed in U.S. Pat. Nos. 6,491,916; 8,663,634; and 9,056,906, each incorporated herein by reference in its entirety. The molecular weight of teplizumab is approximately 150 KD. The full sequences of light and heavy chains are set forth below. Bolded portions are the complementarity determining regions.

Teplizumab Light Chain (SEQ ID NO: 1):
DIQMTQSPSSLSASVGDRVTITCSASSSVSYMNWYQQTPGKAPKRWIY
DTSKLASGVPSRFSGSGSGTDYTFTISSLQPEDIATYYCQQWSSNPFT
FGQGTKLQITRTVAAPSVFIFPPSDEQLKSGTASVVCLLNNFYPREAK
VQWKVDNALQSGNSQESVTEQDSKDSTYSLSSTLTLSKADYEKHKVYA
CEVTHQGLSSPVTKSFNRGEC
Teplizumab Heavy Chain (SEQ ID NO: 2):
QVQLVQSGGGVVQPGRSLRLSCKASGYTFTRYTMHWVRQAPGKGLEWI
GYINPSRGYTNYNQKVKDRFTISRDNSKNTAFLQMDSLRPEDTGVYFC
ARYYDDHYCLDYWGQGTPVTVSSASTKGPSVFPLAPSSKSTSGGTAAL
GCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGGP
SVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHE
ALHNHYTQKSLSLSPGK

In some embodiments, provided herein, is a pharmaceutical composition. Such compositions comprise an effective amount of an anti-CD3 antibody, and a pharmaceutically acceptable carrier. In some embodiments, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like (See, for example, Handbook of Pharmaceutical Excipients, Arthur H. Kibbe (ed., 2000, which is incorporated by reference herein in its entirety), Am. Pharmaceutical Association, Washington, D.C.

The composition, if desired, can also contain minor amounts of wetting or emulsifying agents, or pH buffering agents. These compositions can take the form of solutions, suspensions, emulsion, tablets, pills, capsules, powders, sustained release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions contain a therapeutically effective amount of a therapeutic agent preferably in purified form, together with a suitable amount of carrier so as to provide the form for proper administration to the patient. The formulation should suit the mode of administration. In some embodiments, the pharmaceutical compositions are sterile and in suitable form for administration to a subject, preferably an animal subject, more preferably a mammalian subject, and most preferably a human subject.

In some embodiments, it may be desirable to administer the pharmaceutical compositions locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering the anti-CD3 antibody, care must be taken to use materials to which the anti-CD3 antibody does not absorb.

In some embodiments, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In some embodiments, the composition can be delivered in a controlled release or sustained release system. In some embodiments, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In some embodiments, polymeric materials can be used to achieve controlled or sustained release of the antibodies of the disclosure or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), poly acrylamide, poly(ethylene glycol), polylactides (PLA), poly(lactide-co-glycolides) (PLGA), and polyorthoesters. In some embodiments, the polymer used in a sustained release formulation is inert, free of leachable impurities, stable on storage, sterile, and biodegradable. In some embodiments, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the disclosure or fragments thereof. See, e.g., U.S. Pat. No. 4,526,938; PCT Publication No. WO 91/05548; PCT Publication No. WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

A pharmaceutical composition can be formulated to be compatible with its intended route of administration. Examples of routes of administration include, but are not limited to, parenteral, e.g., intravenous, intradermal, subcutaneous, oral, intranasal (e.g., inhalation), transdermal (topical), transmucosal, and rectal administration. In some embodiments, the composition is formulated in accordance with routine procedures as a pharmaceutical composition adapted for intravenous, subcutaneous, intramuscular, oral, intranasal or topical administration to human beings. In some embodiments, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

The compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In some embodiments, the disclosure provides dosage forms that permit administration of the anti-CD3 antibody continuously over a period of hours or days (e.g., associated with a pump or other device for such delivery), for example, over a period of 1 hour, 2 hours, 3 hours, 4 hours, 6 hours, 8 hours, 10 hours, 12 hours, 16 hours, 20 hours, 24 hours, 30 hours, 36 hours, 4 days, 5 days, 7 days, 10 days or 12 days. In some embodiments, the disclosure provides dosage forms that permit administration of a continuously increasing dose, for example, increasing from 106 μg/m²/day to 850 μg/m²/day or 211 μg/m²/day to 840 μg/m²/day over a period of 24 hours, 30 hours, 36 hours, 4 days, 5 days, 7 days, 10 days or 12 days.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of the compositions disclosed herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In particular, the disclosure provides that the anti-CD3 antibodies, or pharmaceutical compositions thereof, can be packaged in a hermetically sealed container such as an ampoule or sachette indicating the quantity of the agent. In some embodiments, the anti-CD3 antibody, or pharmaceutical compositions thereof is supplied as a dry sterilized lyophilized powder or water free concentrate in a hermetically sealed container and can be reconstituted, e.g., with water or saline to the appropriate concentration for administration to a subject. Preferably, the anti-CD3 antibody, or pharmaceutical compositions thereof is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized agents, or pharmaceutical compositions herein should be stored at between 2° C. and 8° C. in its original container and the therapeutic agents, or pharmaceutical compositions of the disclosure should be administered within 1 week, preferably within 5 days, within 72 hours, within 48 hours, within 24 hours, within 12 hours, within 6 hours, within 5 hours, within 3 hours, or within 1 hour after being reconstituted. In some embodiments, the pharmaceutical composition is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/ml, at least 25 mg/ml, at least 50 mg/ml, at least 75 mg/ml or at least 100 mg/ml. The liquid form should be stored at between 2° C. and 8° C. in its original container.

In some embodiments, the disclosure provides that the composition of the disclosure is packaged in a hermetically sealed container such as an ampoule or sachette indicating the quantity of the anti-CD3 antibody.

The compositions may, if desired, be presented in a pack or dispenser device that may contain one or more unit dosage forms containing the active ingredient. The pack may, for example, comprise metal or plastic foil, such as a blister pack.

The amount of the anti-CD3 antibody or composition comprising the anti-CD3 antibody which is effective in the treatment of one or more symptoms associated with T1D can be determined by standard clinical techniques. The precise dose to be employed in the formulation can also depend on the route of administration and the seriousness of the condition, and should be decided according to the judgment of the practitioner and each patient's circumstances. Effective doses may be extrapolated from dose-response curves derived from in vitro or animal model test systems.

### Dual-Specificity Tyrosine-Regulated Kinase 1A (“DYRK1A”)

Dual-specificity Tyrosine-Regulated Kinase 1A (“DYRK1A”) is a downstream component of a signaling cascade that is able to induce human and rodent beta cell proliferation according to the following scenario (Wang et al., “A High-Throughput Chemical Screen Reveals that Harmine-Mediated Inhibition of DYRK1A Increases Human Pancreatic Beta Cell Replication,” Nat. Med. 21(4):383-388 (2015).

The DYRK1A inhibitor class of drugs include, but are not limited to, harmine (Wang et al., “A High-Throughput Chemical Screen Reveals that Harmine-Mediated Inhibition of DYRK1A Increases Human Pancreatic Beta Cell Replication,” Nat. Med. 21(4):383-388 (2015)), INDY (Wang et al., “A High-Throughput Chemical Screen Reveals that Harmine-Mediated Inhibition of DYRK1A Increases Human Pancreatic Beta Cell Replication,” Nat. Med. 21(4):383-388 (2015)), leucettine (Tahtouh et al., “Selectivity, Co-Crystal Structures and Neuroprotective Properties of Leucettines, a Family of Protein Kinase Inhibitors Derived from the Marine Sponge Alkaloid Leucettamine B,” J. Med. Chem. 55(21):9312-9330 (2012), GNF4877 (Shen et al., “Inhibition of DYRK1A and GSK3B Induces Human Beta Cell Proliferation,” Nat. Commun. 6:8372 (2015)), 5-iodotubericidin (“5-IT”) (Dirice et al., “Inhibition of DYRK1A Stimulates Human Beta Cell Proliferation,” Diabetes 65(6):1660-1671 (2016)); CC-401 (Abdolazimi et al., “CC-401 Promotes Beta Cell Replication via Pleiotropic Consequences of DYRK1A/B Inhibition,” Endocrinology 159(9):3143-3157 (2018)), and others (Wang et al., “A High-Throughput Chemical Screen Reveals that Harmine-Mediated Inhibition of DYRK1A Increases Human Pancreatic Beta Cell Replication,” Nat. Med. 21(4):383-388 (2015); Shen et al., “Inhibition of DYRK1A and GSK3B Induces Human Beta Cell Proliferation,” Nat. Commun. 6:8372 (2015); Dirice et al., “Inhibition of DYRK1A Stimulates Human Beta Cell Proliferation,” Diabetes 65(6):1660-1671 (2016); Abdolazimi et al., “CC-401 Promotes Beta Cell Replication via Pleiotropic Consequences of DYRK1A/B Inhibition,” Endocrinology 159(9):3143-3157 (2018); Aamodt et al., “Development of a Reliable Automated Screening System to Identify Small Molecules and Biologics that Promote Human Beta Cell Regeneration,” AJP Endo. Metab. 311:E859-68 (2016); and Wang et al., “Single Cell Mass Cytometry Analysis of Human Endocrine Pancreas,” Cell Metab. 24(4):616-626 (2016).

Harmine is a derivative compound of ayahuasca, a traditional American medicinal plant which has hallucinogenic effects. Synthetic DYRK1A inhibitors have been recently developed (Kumar et al., J Med Chem. 2020 Mar. 26; 63(6):2986-3003) that are not expected to have hallucinogenic effects.

In some embodiments, the DYRK1A inhibitor comprises or consists of harmine, or harmine derivatives.

In some embodiments, the DYRK1A inhibitor comprises or consists of synthetic DYRK1A inhibitors.

Suitable DYRK1A inhibitors include, but are not limited to, the inhibitors described in PCT Application No. PCT/US2017/058498, filed Oct. 26, 2017, PCT Application No. PCT/US2019/012442, filed Jan. 5, 2019, PCT Application No. PCT/US2018/062023, filed Nov. 20, 2018, PCT Application No. PCT/US2019/069057, filed Dec. 31, 2019, PCT Application No. PCT/US2019/069059, filed Dec. 31, 2019, and PCT Application No. PCT/US2019/023206, filed Mar. 20, 2019, all of which are hereby incorporated by reference in their entireties.

In some embodiments, the DYRK1A inhibitor comprises or consists of small molecule including, but not limited to, epigallocatechin (EGCG) and other flavan-3-ols, leucettines, quinalizarine, peltogynoids Acanilol A and B, benzocoumarins (dNBC), indolocarbazoles (staurosporine, rebeccamycin, and their analogues), INDY, DANDY, and FINDY, pyrazolidinediones, amino-quinazolines, meriolins, pyridine and pyrazines, chromenoidoles, 11H-indolo[3,2-c]quinoline-6-carboxylic acids, thiazolo[5,4-f]quinazolines (EHT 5372), CC-401, and 5-iodotubercidin.

In some embodiments the DYRK1A inhibitors are optionally cross-linked to beta-cells-targeting agents or pancreatic-targeting agents.

The amount of the DYRK1A inhibitors or composition comprising the DYRK1A inhibitors which is effective in the treatment of one or more symptoms associated with T1D can be determined by standard clinical techniques. The precise dose to be administered can depend on the route of administration and the seriousness of the condition. Effective doses may be extrapolated from dose-response curves derived from in vitro or animal model test systems.

Suitable doses include, but are not limited, to those described in PCT Application No. PCT/US2017/058498, filed Oct. 26, 2017, PCT Application No. PCT/US2019/012442, filed Jan. 5, 2019, PCT Application No. PCT/US2018/062023, filed Nov. 20, 2018, PCT Application No. PCT/US2019/069057, filed Dec. 31, 2019, PCT Application No. PCT/US2019/069059, filed Dec. 31, 2019, PCT Application No. PCT/US2019/023206, filed Mar. 20, 2019, which are hereby incorporated by reference in their entireties.

### Methods and Use

In some embodiments, the method of the present disclosure encompasses administration of anti-human CD3 antibodies, such as teplizumab, in combination with DYRK1A inhibitors to patients diagnosed with T1D having a residual beta cell mass. In some embodiments, the patients diagnosed with T1D have a peak C-peptide level of ≥0.2 pmol/mL during a mixed meal tolerance test (MMTT). In some embodiments, the peak C-peptide level at screening ranges from 0.2 pmol/mL (inclusive) to 0.7 pmol/mL (inclusive)

In some embodiments, the present disclosure encompasses administration of anti-human CD3 antibodies, such as teplizumab, in combination with DYRK1A inhibitors to patients 8 through 17 years old 6 weeks from T1D diagnosis having a peak C-peptide level of ≥0.2 pmol/mL during a mixed meal tolerance test (MMTT). In some embodiments, the peak C-peptide level at screening ranges from 0.2 pmol/mL (inclusive) to 0.7 pmol/mL (inclusive).

In some embodiments, T1D diagnosis is according to the American Diabetes Association (ADA) criteria. As defined by the American Diabetes Association (ADA) for the clinical diagnosis of diabetes, the individual must meet one of the following 4 criteria:

A fasting plasma glucose (FPG) of 2126 mg/dL (7.0 mmol/L). Fasting is defined as no caloric intake for at least 8 hours.

A 2-hour plasma glucose (PG) of ≥200 mg/dL (11.1 mmol/L) during an oral glucose tolerance test (OGTT). The test should be performed as described by the World Health Organization (WHO), using a glucose load containing the equivalent of 75 g anhydrous glucose dissolved in water.

A hemoglobin A1C (HbA1c) of ≥6.5% (48 mmol/mol). The test should be performed in a laboratory using a method that is National Glycohemoglobin Standardization Program (NGSP) certified and standardized to the Diabetes Control and Complications Trial (DCCT) assay.

In a patient with classic symptoms of hyperglycemia or hyperglycemic crisis, a random PG of ≥200 mg/dL (11.1 mmol/L).

For the diagnosis of clinical Type 1 diabetes (T1D), the ADA suggests that plasma blood glucose rather than HbA1C should be used to diagnose the acute onset of T1D in individuals with symptoms of hyperglycemia.

According to ADA, a patient with classic symptoms, measurement of plasma glucose is sufficient to diagnose clinical diabetes (symptoms of hyperglycemia or hyperglycemic crisis plus a random plasma glucose≥200 mg/dL [11.1 mmol/L]). In these cases, knowing the plasma glucose level is critical because, in addition to confirming that symptoms are due to diabetes, it will inform management decisions. Some providers may also want to know the HbA1C to determine how long a patient has had hyperglycemia. In addition, T1D, previously called “insulin-dependent diabetes” or “juvenile-onset diabetes,” accounts for 5-10% of diabetes and is due to cellular-mediated autoimmune destruction of the pancreatic β-cells. Autoimmune markers include islet cell autoantibodies and autoantibodies to GAD (GAD65), insulin, the tyrosine phosphatases IA-2 and IA-2 β, and ZnT8. T1D is defined by the presence of one or more of these autoimmune markers.

In some embodiments, the patient diagnosed with clinical T1D has a positive result on testing for at least one of the following T1D-related autoantibodies: Glutamic acid decarboxylase 65 (GAD65) autoantibodies, Islet antigen 2 (IA-2) autoantibodies, Zinc transporter 8 (ZnT8) autoantibodies Islet cell cytoplasmic autoantibodies (ICA) or Insulin autoantibodies (if testing obtained within the first 14 days of insulin treatment). In some embodiments, the presence of the autoantibodies is detected by ELISA, electrochemoluminescence (ECL), radioassay (see, e.g., Yu et al., 1996, J. Clin. Endocrinol. Metab. 81:4264-4267), agglutination PCR (Tsai et al, ACS Central Science 2016 2 (3), 139-147) or by any other method for immunospecific detection of antibodies described herein or as known to one of ordinary skill in the art.

It is recognized that β cells continue to be lost following T1D diagnosis. To maximize the effect of β cell preservation in patients with a recoverable level endogenous insulin production, the patients to be treated are within 6 weeks from T1D diagnosis and have a peak C-peptide level of ≥0.2 pmol/mL during a mixed meal tolerance test (MMTT).

In some embodiments, the methods provided herein prevents or delays the need for administration of exogenous insulin to the patients.

β-cell function prior to, during, and after therapy may be assessed by methods described herein or by any method known to one of ordinary skill in the art. For example, the Diabetes Control and Complications Trial (DCCT) research group has established the monitoring of percentage glycosylated hemoglobin (HA1 and HA1c) as the standard for evaluation of blood glucose control (DCCT, 1993, N. Engl. J. Med. 329:977-986). Alternatively, characterization of daily insulin needs, C-peptide levels/response, hypoglycemic episodes, and/or FPIR may be used as markers of β-cell function or to establish a therapeutic index (See Keymeulen et al., 2005, N. Engl. J. Med. 352:2598-2608; Herold et al., 2005, Diabetes 54:1763-1769; U.S. Pat. Appl. Pub. No. 2004/0038867 A1; and Greenbaum et al., 2001, Diabetes 50:470-476, respectively). For example, FPIR is calculated as the sum of insulin values at 1 and 3 minutes post IGTT, which are performed according to Islet Cell Antibody Register User's Study protocols (see, e.g., Bingley et al., 1996, Diabetes 45:1720-1728 and McCulloch et al., 1993, Diabetes Care 16:911-915).

In some embodiments, the method comprises a 8 to 16-day course of administration of anti-CD3 antibodies. In some embodiments, anti-CD3 antibodies can be administered orally, by intravenous injection, intramuscular injection, subcutaneous injection, intraperitoneal injection, topical, sublingual, intraarticular, intradermal, buccal, ophthalmic (including intraocular), intranasally or by any other routes. Anti-CD3 antibodies can be administered orally, e.g., as a tablet or cachet containing a predetermined amount of the anti-CD3 antibodies, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation, via any formulations known in the art or in some other form.

In some embodiments, the effective amount comprises a 12-day course of subcutaneous intravenous (IV) infusion of the anti-CD3 antibody such as teplizumab at 106-850 micrograms/meter squared (μg/m²). In some embodiments, the total dosage over the duration of the regimen is about 14000 μg/m², 13500 μg/m², 13000 μg/m², 12500 μg/m², 12000 μg/m², 11500 μg/m², 11000 μg/m², 10500 μg/m², 10000 μg/m², 9500 μg/m², 9000 μg/m², 8000 μg/m², 7000 μg/m², 6000 μg/m², and may be less than 5000 μg/m², 4000 μg/m², 3000 μg/m², 2000 μg/m², or 1000 μg/m². In some embodiments, the total dosage over the duration of the regimen is from about 9030 μg/m² to about 14000 μg/m², about 9030 μg/m² to about 13500 μg/m², about 9000 μg/m² to about 13000 μg/m², about 9000 μg/m² to about 12500 μg/m², about 9000 μg/m² to about 12000 μg/m², about 9000 μg/m² to about 11500 μg/m², about 9000 μg/m² to about 11000 μg/m², about 9000 μg/m² to about 10500 μg/m², about 9000 μg/m² to about 10000 μg/m², about 9000 μg/m² to about 9500 μg/m². In some embodiments, the total dosage over the duration of the regimen is from about 9030 μg/m² to about 14000 μg/m², about 9030 μg/m² to about 13500 μg/m², about 9030 μg/m² to about 13000 μg/m², about 9030 μg/m² to about 12500 μg/m², about 9030 μg/m² to about 12000 μg/m², about 9030 μg/m² to about 11500 μg/m², from about 9030 μg/m² to about 11000 μg/m², about 9030 μg/m² to about 10500 μg/m², about 9030 μg/m² to about 10000 μg/m², about 9030 μg/m² to about 9500 μg/m².

In some embodiments, the effective amount comprises a 12-day course of subcutaneous intravenous (IV) infusion of the anti-CD3 antibody such as teplizumab at 106-850 micrograms/meter squared (μg/m²). In some embodiments, the effective amount comprises a 12-day course IV infusion of teplizumab at a first dose of 106 μg/m² teplizumab on day 1, a second dose of 425 μg/m² teplizumab on day 2, and one dose of 850 μg/m² teplizumab on each of days 3-12. In some embodiments, the effective amount comprises a 12-day course IV infusion of teplizumab at a first dose of 211 μg/m² teplizumab on day 1, a second dose of 423 μg/m² teplizumab on day 2, and one dose of 840 μg/m² teplizumab on each of days 3-12.

In some embodiments, the effective amount comprises a 12-day course IV infusion of teplizumab at a first dose of approximately 100 μg/m² teplizumab on day 1, a second dose of approximately 400 μg/m² teplizumab on day 2, a third dose of approximately 850 μg/m² teplizumab on day 3, and approximately 1200 μg/m² teplizumab on each of days 4-12. In some embodiments, the effective amount comprises a 12-day course IV infusion of teplizumab at a first dose of approximately 100 μg/m² teplizumab on day 1, a second dose of approximately 400 μg/m² teplizumab on day 2, a third dose of approximately 850 μg/m² teplizumab on day 3, and approximately 1300 μg/m² teplizumab on each of days 4-12. In some embodiments, the effective amount comprises a 12-day course IV infusion of teplizumab at a first dose of approximately 100 μg/m² teplizumab on day 1, a second dose of approximately 400 μg/m² teplizumab on day 2, a third dose of approximately 850 μg/m² teplizumab on day 3, and approximately 1400 μg/m² teplizumab on each of days 4-12. In some embodiments, each 12-day course can include a 2-day ramp-up phase and a 10-day fixed, maximal dosing period. In some embodiments, 106 μg/m² teplizumab is administered on day 1425 μg/m² teplizumab is administered on day 2, and 850 μg/m² teplizumab is administered on each of days 3-12.

In some embodiments, the effective amount comprises a 12-day course IV infusion of teplizumab at a first dose of approximately 200 μg/m² teplizumab on day 1, a second dose of approximately 400 μg/m² teplizumab on day 2, a third dose of approximately 850 μg/m² teplizumab on day 3, and approximately 1200 μg/m² teplizumab on each of days 4-12. In some embodiments, the effective amount comprises a 12-day course IV infusion of teplizumab at a first dose of approximately 200 μg/m² teplizumab on day 1, a second dose of approximately 400 μg/m² teplizumab on day 2, a third dose of approximately 850 μg/m² teplizumab on day 3, and approximately 1300 μg/m² teplizumab on each of days 4-12. In some embodiments, the effective amount comprises a 12-day course IV infusion of teplizumab at a first dose of approximately 200 μg/m² teplizumab on day 1, a second dose of approximately 400 μg/m² teplizumab on day 2, a third dose of approximately 850 μg/m² teplizumab on day 3, and approximately 1400 μg/m² teplizumab on each of days 4-12.

Provided herein is a dosing regimen comprising two or more courses of dosing with an anti-CD3 antibody such as teplizumab comprising a first course of dosing at week 1 and second course of dosing at week 26. In some embodiments, teplizumab is administered via IV infusion in two courses, with the first course starting on Day 1 (Week 1) and the second course on approximately Day 182 (Week 26), each course of treatment including daily infusions for 12 days, with a cumulative teplizumab dose of 9000 μg/m² for each course of treatment. In some embodiments, the 12 days course has a 2-day ramp-up phase and a 10-day fixed-, maximal dosing period. In some embodiments, 106 μg/m² teplizumab is administered on day 1, 425 μg/m² teplizumab is administered on day 2, and 850 μg/m² teplizumab is administered on each of days 3-12. In some embodiments, teplizumab is administered via IV infusion in two courses, with the first course starting on Day 1 (Week 1) and the second course on approximately Day 182 (Week 26), each course of treatment including daily infusions for 12 days, with a cumulative teplizumab dose of 9500 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 12 days, with a cumulative teplizumab dose of 10000 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 12 days, with a cumulative teplizumab dose of 10500 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 12 days, with a cumulative teplizumab dose of 11000 μg/m² for each course In some embodiments, teplizumab is administered via IV infusion in two courses, with the first course starting on Day 1 (Week 1) and the second course on approximately Day 182 (Week 26), each course of treatment including daily infusions for 12 days, with a cumulative teplizumab dose of 11500 μg/m² for each course of treatment. of treatment. In some embodiments, teplizumab is administered via IV infusion in two courses, with the first course starting on Day 1 (Week 1) and the second course on approximately Day 182 (Week 26), each course of treatment including daily infusions for 12 days, with a cumulative teplizumab dose of 12000 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 12 days, with a cumulative teplizumab dose of 12500 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 12 days, with a cumulative teplizumab dose of 13000 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 12 days, with a cumulative teplizumab dose of 13500 μg/m² for each course of treatment. In some embodiments, teplizumab is administered via IV infusion in two courses, with the first course starting on Day 1 (Week 1) and the second course on approximately Day 182 (Week 26), each course of treatment including daily infusions for 12 days, with a cumulative teplizumab dose of 14000 μg/m² for each course of treatment. In some embodiments, the 12 days course has a 2-day ramp-up phase and a 10-day fixed-, maximal dosing period. In some embodiments, 106 μg/m² teplizumab is administered on day 1425 μg/m² teplizumab is administered on day 2, and 850 μg/m² teplizumab is administered on each of days 3-12.

In other embodiments, the course of dosing can be repeated at 2 month, 3 month, 4 month, 5 month, 6 month, 8 month, 9 month, 10 month, 12 month, 15 month, 18 month, 24 month, 30 month, or 36 month intervals. In some embodiments, efficacy of the treatment with the anti-CD3 antibody such as teplizumab is determined as described herein, or as is known in the art, at 2 months, 3 months, 4 months, 5 month, 6 months, 9 months, 12 months, 15 months, 18 months, 24 months, 30 months, or 36 months subsequent to the previous treatment.

In some embodiments, a subject is administered one or more doses, preferably 12 daily doses, of the anti-CD3 antibody such as teplizumab at about 5-1200 μg/m², preferably, 106-850 μg/m² to treat, or slow the progression of or ameliorate one or more symptoms of T1D.

In some embodiments, the subject is administered a treatment regimen comprising two courses of daily doses of an effective amount of the anti-CD3 antibody such as teplizumab, wherein the course of treatment is administered over 2 days, 3 days, 4 days, 5 days, 6 days, 7 days, 8 days, 9 days, 10 days, 11 days or 12 days. In some embodiments, the treatment regimen comprises administering doses of the effective amount every day, every 2nd day, every 3rd day or every 4th day.

In some embodiments, a subject is administered a treatment regimen comprising one or more doses of an effective amount of the anti-CD3 antibody such as teplizumab, wherein the effective amount is 200 ug/kg/day, 175 ug/kg/day, 150 ug/kg/day, 125 ug/kg/day, 100 ug/kg/day, 95 ug/kg/day, 90 ug/kg/day, 85 ug/kg/day, 80 ug/kg/day, 75 ug/kg/day, 70 ug/kg/day, 65 ug/kg/day, 60 ug/kg/day, 55 ug/kg/day, 50 ug/kg/day, 45 ug/kg/day, 40 ug/kg/day, 35 ug/kg/day, 30 ug/kg/day, 26 ug/kg/day, 25 ug/kg/day, 20 ug/kg/day, 15 ug/kg/day, 13 ug/kg/day, 10 ug/kg/day, 6.5 ug/kg/day, 5 ug/kg/day, 3.2 ug/kg/day, 3 ug/kg/day, 2.5 ug/kg/day, 2 ug/kg/day, 1.6 ug/kg/day, 1.5 ug/kg/day, 1 ug/kg/day, 0.5 ug/kg/day, 0.25 ug/kg/day, 0.1 ug/kg/day, or 0.05 ug/kg/day; and/or wherein the prophylactically effective amount is 1200 ug/m²/day, 1150 ug/m²/day, 1100 ug/m²/day, 1050 ug/m²/day, 1000 ug/m²/day, 950 ug/m²/day, 900 ug/m²/day, 850 ug/m²/day, 800 ug/m²/day, 750 ug/m²/day, 700 ug/m²/day, 650 ug/m²/day, 600 ug/m²/day, 550 ug/m²/day, 500 ug/m²/day, 450 ug/m²/day, 400 ug/m²/day, 350 ug/m²/day, 300 ug/m²/day, 250 ug/m² day, 200 ug/m²/day, 150 ug/m²/day, 100 ug/m²/day, 50 ug/m²/day, 40 ug/m² day, 30 ug/m²/day, 20 ug/m²/day, 15 ug/m²/day, 10 ug/m²/day, or 5 ug/m²/day.

In some embodiments, the intravenous dose of 1400 μg/m² or less, 1350 μg/m² or less, 1300 μg/m² or less, 1250 μg/m² or less 1200 μg/m² or less, 1150 μg/m² or less, 1100 μg/m² or less, 1050 μg/m² or less, 1000 μg/m² or less, 950 μg/m² or less, 900 μg/m² or less, 850 μg/m² or less, 800 μg/m² or less, 750 μg/m² or less, 700 μg/m² or less, 650 μg/m² or less, 600 μg/m² or less, 550 μg/m² or less, 500 μg/m² or less, 450 μg/m² or less, 400 μg/m² or less, 350 μg/m² or less, 300 μg/m² or less, 250 μg/m² or less, 200 μg/m² or less, 150 μg/m² or less, 100 μg/m² or less, 50 μg/m² or less, 40 μg/m² or less, 30 μg/m² or less, 20 μg/m² or less, 15 μg/m² or less, 10 μg/m² or less, or 5 μg/m² or less of the anti-CD3 antibody such as teplizumab is administered over about 24 hours, about 22 hours, about 20 hours, about 18 hours, about 16 hours, about 14 hours, about 12 hours, about 10 hours, about 8 hours, about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat or ameliorate one or more symptoms of type 1 diabetes. The total dosage over the duration of the regimen is preferably a total of less than about 14000 μg/m², 13500 μg/m², 13000 μg/m², 12500 μg/m², 12000 μg/m², 11500 μg/m², 11000 μg/m², 10500 μg/m², 10000 μg/m², 9500 μg/m², 9000 μg/m², 8000 μg/m², 7000 μg/m², 6000 μg/m², and may be less than 5000 μg/m², 4000 μg/m², 3000 μg/m², 2000 μg/m², or 1000 μg/m². In some embodiments, the total dosage administered in the regimen is 100 μg/m² to 200 μg/m², 100 μg/m² to 500 μg/m²², 100 μg/m² to 1000 μg/m², or 500 μg/m² to 1000 μg/m².

In some embodiments, the dose escalates over the first three, first 1/4 of the doses (e.g., over the first 3 days of a 12-day regimen of one dose per day) of the treatment regimen until the daily effective amount of the anti-CD3 antibody such as teplizumab is achieved. In some embodiments, a subject is administered a treatment regimen comprising one or more doses of an effective amount of the anti-CD3 antibody such as teplizumab, wherein the effective amount is increased by, e.g., 0.01 ug/kg, 0.02 ug/kg, 0.04 ug/kg, 0.05 ug/kg, 0.06 ug/kg, 0.08 ug/kg, 0.1 ug/kg, 0.2 ug/kg, 0.25 ug/kg, 0.5 ug/kg, 0.75 ug/kg, 1 ug/kg, 1.5 ug/kg, 2 ug/kg, 4 ug/kg, 5 ug/kg, 10 ug/kg, 15 ug/kg, 20 ug/kg, 25 ug/kg, 30 ug/kg, 35 ug/kg, 40 ug/kg, 45 ug/kg, 50 ug/kg, 55 ug/kg, 60 ug/kg, 65 ug/kg, 70 ug/kg, 75 ug/kg, 80 ug/kg, 85 ug/kg, 90 ug/kg, 95 ug/kg, 100 ug/kg, or 125 ug/kg each day; or increased by, e.g., 100 ug/m², 150 ug/m², 200 ug/m², 250 ug/m², 300 ug/m², 350 ug/m², 400 ug/m², 450 ug/m², 500 ug/m², 550 ug/m², 600 ug/m², or 650 ug/m², each day as treatment progresses. In some embodiments, a subject is administered a treatment regimen comprising one or more doses of a effective amount of the anti-CD3 antibody such as teplizumab, wherein the effective amount is increased by a factor of 1.25, a factor of 1.5, a factor of 2, a factor of 2.25, a factor of 2.5, or a factor of 5 until the daily effective amount of the anti-CD3 antibody such as teplizumab is achieved.

In some embodiments, a subject is intramuscularly administered one or more doses of a 200 μg/kg or less, preferably 175 μg/kg or less, 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 ug/kg or less, 2 μg/kg or less, 1.5 ug/kg or less, 1 μg/kg or less, 0.5 ug/kg or less, or 0.2 ug/kg or less of the anti-CD3 antibody such as teplizumab to treat or ameliorate one or more symptoms of T1D.

In some embodiments, a subject is subcutaneously administered one or more doses of a 200 ug/kg or less, preferably 175 ug/kg or less, 150 ug/kg or less, 125 ug/kg or less, 100 ug/kg or less, 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab to treat or ameliorate one or more symptoms of T1D.

In some embodiments, a subject is intravenously administered one or more doses of a 100 ug/kg or less, preferably 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab to treat or ameliorate one or more symptoms of T1D. In some embodiments, the intravenous dose of 100 ug/kg or less, 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, is administered over about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to treat or ameliorate one or more symptoms of T1D.

In some embodiments, a subject is orally administered one or more doses of a 100 ug/kg or less, preferably 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab to treat or ameliorate one or more symptoms of T1D. In some embodiments, the oral dose of 100 ug/kg or less, 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab is administered over about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to treat or ameliorate one or more symptoms of T1D.

In some embodiments in which escalating doses are administered for the first days of the dosing regimen, the dose on day 1 of the regimen is 100-250 μg/m²/day, preferably 106 μg/m²/day and escalates to the daily dose as recited immediately above by day 2, and 3. For example, on day 1, the subject is administered a dose of approximately 106 μg/m²/day, on day 2 approximately 425 μg/m²/day, and on subsequent days of the regimen (e.g., days 3-12) 850 ug/m²/day. In some embodiments, on day 1, the subject is administered a dose of approximately 211 ug/m²/day, on day 2 approximately 423 μg/m²/day, on day 3 and subsequent days of the regimen (e.g., days 3-12) approximately 840 μg/m²/day.

In some embodiments, to reduce the possibility of cytokine release and other adverse effects, the first 1, 2, or 3 doses or all the doses in the regimen are administered more slowly by intravenous administration. For example, a dose of 106 μg/m²/day may be administered over about 5 minutes, about 15 minutes, about 30 minutes, about 45 minutes, about 1 hour, about 2 hours, about 4 hours, about 6 hours, about 8 hours, about 10 hours, about 12 hours, about 14 hours, about 16 hours, about 18 hours, about 20 hours, and about 22 hours. In some embodiments, the dose is administered by slow infusion over a period of, e.g., 20 to 24 hours. In some embodiments, the dose is infused in a pump, preferably increasing the concentration of antibody administered as the infusion progresses.

In some embodiments, a set fraction of the doses for the 106 μg/m²/day to 850 μg/m²/day regimen described above is administered in escalating doses.

In some embodiments, the anti-CD3 antibody such as teplizumab is not administered by daily doses over a number of days, but is rather administered by infusion in an uninterrupted manner over 4 hours, 6 hours, 8 hours, 10 hours, 12 hours, 15 hours, 18 hours, 20 hours, 24 hours, 30 hours or 36 hours. The infusion may be constant or may start out at a lower dosage for, for example, the first 1, 2, 3, 5, 6, or 8 hours of the infusion and then increase to a higher dosage thereafter. Over the course of the infusion, the patient receives a dose equal to the amount administered in the 5 to 20-day regimens set forth above. For example, a dose of approximately 150 μg/m², 200 μg/m², 250 μg/m², 500 μg/m², 750 μg/m², 1000 μg/m², 1500 μg/m², 2000 μg/m², 3000 μg/m², 4000 μg/m², 5000 μg/m², 6000 μg/m², 7000 μg/m², 8000 μg/m², 9000 μg/m², 9500 μg/m², 10000 μg/m², 10500 μg/m², 11000 μg/m², 11500 μg/m², 12000 μg/m², 12500 μg/m², 13000 μg/m², 13500 μg/m² or 14000 μg/m² can be administered. In particular, the speed and duration of the infusion is designed to minimize the level of free anti-CD3 antibody such as teplizumab in the subject after administration. In some embodiments, the level of free anti-CD3 antibody such as teplizumab should not exceed 200 ng/ml free antibody. In addition, the infusion is designed to achieve a combined T cell receptor coating and modulation of at least 50%, 60%, 70%, 80%, 90%, 95% or of 100%.

In some embodiments, the anti-CD3 antibody such as teplizumab is administered chronically to treat, or slow the progression, or ameliorate one or more symptoms of type 1 diabetes. For example, in some embodiments, a low dose of the anti-CD3 antibody such as teplizumab is administered once a month, twice a month, three times per month, once a week or even more frequently either as an alternative to the 6 to 14-day dosage regimen discussed above or after administration of such a regimen to enhance or maintain its effect. Such a low dose may be anywhere from 1 μg/m² to 100 μg/m², such as approximately 5 μg/m², 10 μg/m², 15 μg/m², 20 μg/m², 25 μg/m², 30 μg/m², 35 μg/m², 40 μg/m², 45 μg/m², or 50 μg/m².

In some embodiments, the subject may be re-dosed at some time subsequent to administration of the two course anti-CD3 antibody such as teplizumab dosing regimen, for example, based upon one or more physiological or biomarker parameters or may be done as a matter of course. Such redosing may be administered and/or the need for such redosing evaluated 2 months, 4 months, 6 months, 8 months, 9 months, 1 year, 15 months, 18 months, 2 years, 30 months or 3 years after administration of a dosing regimen and may include administering a course of treatment every 6 months, 9 months, 1 year, 15 months, 18 months, 2 years, 30 months or 3 years or indefinitely.

In some embodiments, before and/or after (e.g., at 1-6 month interval, or 2-5 month interval, or about 3 month interval) the administration of a 12-day course of teplizumab, the level (or relative amounts) of phenotypically exhausted T cells, such as TIGIT+KLRG1+CD8+CD3+ cells with respect to all CD3+ T cells is determined, for example by flow cytometry. In some embodiments, the level of the TIGIT+KLRG1+CD8+CD3+ T-cells can be monitored for example by flow cytometry. In some embodiments, an additional 12-day course of anti-CD3 antibody, such as teplizumab, is administered when the level of the TIGIT+KLRG1+CD8+CD3+ T-cells corresponds to (e.g., returns to) the baseline level. In some embodiments, the determining of TIGIT+KLRG1+CD8+CD3+ T-cells is about 3 months (or about 1-6 months) after the administration of the second 12-day course. In some embodiments, if the subject has more than about 10% TIGIT+KLRG1+CD8+ T-cells in all CD3+ T cells, the monitoring can be annual. In some embodiments, if the subject has less than about 10% TIGIT+KLRG1+CD8+ T-cells in all CD3+ T cells, the monitoring can be every about 3-6 months.

In some embodiments, the re-dosing comprises administering additional (e.g., second, third, or beyond) 12-day course(s) of teplizumab each at a total dose of more than about 9000 μg/m². In some embodiments, the re-dosing comprises administering additional (e.g., second, third, or beyond) 12-day course(s) of teplizumab each at a total dose of between about 9000 and about 14000 μg/m². In some embodiments, the additional 12-day course of teplizumab comprises a first dose of 106 μg/m² teplizumab on day 1, a second dose of 425 μg/m² teplizumab on day 2, and one dose of 850 μg/m² on each of days 3-12, and wherein the total dose is approximately 9031 μg/m². In other embodiments the additional 12-day course of teplizumab comprises a first dose of 211 μg/m² teplizumab on day 1, a second dose of 423 μg/m² teplizumab on day 2, and one dose of 840 μg/m² on each of days 3-12, and wherein the total dose is approximately 9034 pig/m².

In some embodiments, the additional (e.g., second, third, or beyond) 12-day course of anti-CD3 antibody, such as teplizumab, can be administered about 12 month to about a 24 month after the administering of the prior 12-day course, for example 12, 13, 14, 15, 16, 17, 19, 20, 21, 22, 23 or 24 months.

In some embodiments, the effective amount of the antibody comprises a 10 to 14 day course of subcutaneous (SC) injection or intravenous (IV) infusion or oral administration of an effective amount of anti-CD3 antibody at a cumulative dose greater than 10,000 micrograms/meter squared (μg/m²). In some embodiments, the effective amount comprises a 10 to 14-day course of subcutaneous (SC) injection or intravenous (IV) infusion or oral administration of the anti-CD3 antibody at from about 9500 to about 14000 μg/m², from about 9500 to about 13500 μg/m², from about 9500 to about 13000 μg/m², from about 9,500 to about 12500 μg/m², from about 9500 to about 12000 μg/m², from about 9500 to about 11500 μg/m², from about 9500 to about 11,000 μg/m², from about 9500 to about 10500 μg/m², from about 9500 to about 10000 μg/m². For example, the total dosage over the duration of the regimen is about 10935 μg/m², 11235 μg/m², 11640 μg/m², or 12225 μg/m². In some embodiments, the effective amount of the antibody comprises a 10 to 14 day course of subcutaneous (SC) injection or intravenous (IV) infusion or oral administration of the anti-CD3 antibody at about 10000 μg/m², 10500 μg/m², 11000 μg/m², 11500 μg/m², 12000 μg/m², 12500 μg/m², 13000 μg/m², 13500 μg/m², or 14000 μg/m². In some embodiments, the anti-CD3 antibody is or comprises teplizumab.

In some embodiments, the anti-CD3 antibody is administered prophylactically to non-diabetic subject who is at risk for T1D. In some embodiments, the effective amount comprises a 10 to 14 day course of subcutaneous (SC) injection or intravenous (IV) infusion or oral administration of the anti-CD3 antibody at a cumulative dose greater than 10000 μg/m².

In some embodiments, the method comprises administering to the non-diabetic subject who is at risk for T1D a 14-day course IV infusion at about 60 μg/m², about 125 μg/m², about 250 μg/m², and about 500 μg/m², on days 1-4, respectively, and one dose of about 1000 μg/m² on each of days 5-14. In some embodiments, the cumulative dose is about 10935 μg/m².

In some embodiments, the method comprises administering to the non-diabetic subject who is at risk for T1D a 14-day course IV infusion at about 60 μg/m², about 125 μg/m², about 250 μg/m², and about 500 μg/m², on days 1-4, respectively, and one dose of about 1030 μg/m² on each of days 5-14. In some embodiments, the cumulative dose is about 11235 μg/m².

In some embodiments, the method comprises administering to the non-diabetic subject who is at risk for T1D a 14-day course IV infusion at about 100 μg/m², about 425 μg/m², about 850 μg/m², and about 850 μg/m², on days 1-4, respectively, and one dose of about 1000 μg/m² on each of days 5-14. In some embodiments, the cumulative dose is about 12225 μg/m².

In some embodiments, the method comprises administering to the non-diabetic subject who is at risk for T1D a 14-day course IV infusion at about 65 μg/m², about 125 μg/m², about 250 μg/m², and about 500 μg/m², on days 1-4, respectively, and one dose of about 1,070 μg/m² on each of days 5-14. In some embodiments, the cumulative dose is about 11640 μg/m².

In some embodiments, the anti-CD3 antibody such as teplizumab is administered to achieve, or maintain a level of glycosylated hemoglobin (HA1 or HA1 c) less than 8%, less than 7.5%, less than 7%, less than 6.5%, less than 6%, less than 5.5% or 5% or less. At the initiation of treatment, patients have a HA1 or HAIc level of less than 8%, less than 7.5%, less than 7%, less than 6.5%, less than 6%, or, more preferably, from 4%-6% (preferably measured in the absence of other treatment for diabetes, such as administration of exogenous insulin). Such patients preferably have retained at least 95%, 90%, 80%, 70%, 60%, 50%, 40% 30% or 20% of beta-cell function prior to initiation of treatment. In some embodiments, the administration of the anti-CD3 antibodies prevents damage, thereby slowing progression of the disease and reducing the need for insulin administration. In some embodiments, the methods of treatment provided herein result in a level of HA1 or HA1c is 7% or less, 6.5% or less, 6% or less, 5.5% or less, or 5% or less 6 months, 9 months, 12 months, 15 months, 18 months, or 24 months after the previous treatment. In some embodiments, the administration of the anti-CD3 antibodies according to the methods provided herein decreases the average level of HA1 or HA1c in the patient by about 5%, about 10%, about 15%, about 20%, about 25%, about 30%, about 35%, about 40%, about 45%, about 50%, about 55%, about 60%, about 65% or about 70% as compared to pre-treatment levels at 6 months, 9 months, 12 months, 15 months, 18 months, or 24 months after the previous treatment. In some embodiments, the administration of the anti-CD3 antibodies according to the methods provided herein results in an average level of HA1 or HA1c in the patient that only increases by about 0.5%, about 1%, about 2.5%, about 5%, about 10%, about 15%, about 20%, about 25%, about 30%, about 35%, about 40%, about 45%, or about 50% as compared to pre-treatment levels at 6 months, 9 months, 12 months, 15 months, 18 months, or 24 months after the previous treatment.

In some embodiments, administration of the anti-CD3 antibodies, in particular teplizumab according to the methods provided herein slows the loss of β cells and/or preserves 3 cell function (as evidenced by e.g., C-peptide levels, episodes of hypo- or hyper-glycemia, time in range (of glycemia), insulin use, or other assessment method known in the art) over 12 months, 13 months, 14 months, 15 months, 16 months, 17 months, 18 months, 19 months, 20 months, 21 months, 22 months, 2 months, 24 month or more in children and adolescents 8-17 years old who have been diagnosed with T1D in the previous 6 weeks. In some embodiments, administration of the anti-CD3 antibodies, in particular teplizumab according to the methods provided herein slows the loss of β cells and/or preserves β cell function over 18 months (78 weeks) in children and adolescents 8-17 years old who have been diagnosed with T1D in the previous 6 weeks.

In some embodiments, the method comprises administering an effective amount of one or more anti-CD3 antibody, for example teplizumab, in combination with an effective amount of one or more DYRK1A inhibitor.

In some embodiments, the effective amount of DYRK1A inhibitor(s) is administered daily for at least 3 months, for example 3, 4, 5, 6, 7, 8, 9, 10, 11, 12, 13, 14, 15, 16, 17, 19, 20, 21, 22, 23 or 24 months or more. In some embodiments, the effective amount of DYRK1A inhibitor(s) is administered once every few days (e.g. two days, three days, four days, five days, or seven days) or once weekly.

In some embodiments, the effective amount of DYRK1A inhibitor(s) is administered as a monthly treatment once per month, or daily for the first week of the month or daily on the first 14 days, or daily every other week (1 week on treatment and 1 week off).

Alternatively, treatment of T1D may comprise multiple administrations of an effective dose of the DYRK1A inhibitor(s) carried out over a range of time periods, such as four times daily, three times daily, twice daily, daily, once every few days, weekly, monthly or yearly. As a non-limiting example, a combination disclosed herein can be administered once or twice weekly to a patient. The timing of administration can depend upon factors as the severity of the patient's symptoms. For example, an effective dose of the DYRK1A inhibitor(s) can be administered to a patient once every few dates for an indefinite period of time, or until the patient no longer requires therapy.

In some embodiments, the effective amount of DYRK1A inhibitor(s) is administered until evidence of durable beta cell regeneration is obtained. In some embodiments, regeneration of beta cells can be assessed by HbA1c, glycemia, spot C-peptide or OGTT-stimulated C-peptide, in all cases showing values improved (e.g. closer to normal range) compared to the levels prior to therapy.

In some embodiments, the effective amount of DYRK1A inhibitor(s) is administered orally, intraperitoneally, subcutaneously, or by infusion using a pump or any other delivery device known in the art.

IN some embodiments, the anti-CD3 antibodies, such as teplizumab, and the DYRK1A inhibitor(s) are co-administered. In some embodiments, the anti-CD3 antibodies, such as teplizumab, and the DYRK1A inhibitor(s) are administered simultaneously. In some embodiments, the anti-CD3 antibodies, such as teplizumab, and the DYRK1A inhibitor(s) are administered sequentially.

In some embodiments, the combination of anti-CD3 antibody, such as teplizumab, and DYRK1A inhibitor(s) can be formulated as separate compositions which are given at substantially the same time. In some embodiments, the combination of anti-CD3 antibody and DYRK1A inhibitor(s) can be formulated as a single composition such that all of the active agents are administered at a therapeutically effective amount to the subject in need thereof in each dose.

In some embodiments, the combination of anti-CD3 antibody and DYRK1A inhibitor(s) can be administered to the subject in need thereof at different times. The DYRK1A inhibitors may be administered as a single dose daily or as multiple doses daily.

In some embodiments, a pharmaceutical composition comprising anti-CD-3 antibodies such as teplizumab, is administered intravenously and a pharmaceutical composition comprising DYRK1A inhibitor(s) is administered orally, intraperitoneally, subcutaneously, or by infusion using a pump or any other delivery device known in the art. In some embodiments, a pharmaceutical composition comprising anti-CD-3 antibodies (e.g. teplizumab) is administered subcutaneously and a pharmaceutical composition comprising DYRK1A inhibitor(s) is administered orally, intraperitoneally, subcutaneously, or by infusion using a pump or any other delivery device known in the art. In some embodiments, a pharmaceutical composition comprising anti-CD-3 antibodies (e.g. teplizumab) is administered orally and a pharmaceutical composition comprising DYRK1A inhibitor(s) is administered orally, intraperitoneally, subcutaneously, or by infusion using a pump or any other delivery device known in the art. In some embodiments, a pharmaceutical composition comprising a combination of anti-CD-3 antibodies (e.g. teplizumab) and DYRK1A inhibitor(s) is administered intravenously. In some embodiments, a pharmaceutical composition comprising anti-CD-3 antibodies and a pharmaceutical composition comprising DYRK1A inhibitor(s) are administered intravenously as separate pharmaceutical compositions. In some embodiments, a pharmaceutical composition comprising a combination of anti-CD-3 antibodies (e.g. teplizumab) and DYRK1A inhibitor(s) is administered subcutaneously. In some embodiments, a pharmaceutical composition comprising anti-CD-3 antibodies and a pharmaceutical composition comprising DYRK1A inhibitor(s) are administered subcutaneously as separate pharmaceutical compositions. a pharmaceutical composition comprising a combination of anti-CD-3 antibodies (e.g. teplizumab) and DYRK1A inhibitor(s) is administered orally. In some embodiments, a pharmaceutical composition comprising anti-CD-3 antibodies, such as teplizumab, and a pharmaceutical composition comprising DYRK1A inhibitor(s) are administered orally as separate pharmaceutical compositions.

In some embodiments, the methods provided herein comprise administering to the subject in need thereof, after completion of the course of teplizumab, a therapeutically effective amount of DYRK1A inhibitor(s) until the subject's need for exogenous insulin is reduced by at least 20% or fully eliminated. In some embodiments, the therapeutically effective amount of DYRK1A inhibitor(s) is administered from once daily to once every month (e.g. once weekly, once every two weeks etc.). In some embodiments, the therapeutically effective amount of DYRK1A inhibitor(s) is administered orally, subcutaneously, or intravenously.

In some embodiments, the methods provided herein comprise administering to the subject in need thereof a weekly, once every two weeks, once every three weeks to monthly subcutaneous effective amount of DYRK1A inhibitor(s) after the first course of anti-CD-3 antibodies, such as teplizumab. In some embodiments, the method further comprises administering to the subject in need thereof one or more additional course of the combined anti-CD3 antibodies and DYRK1A inhibitor(s). In some embodiments, the additional course of anti-CD3 antibodies and DYRK1A inhibitor(s) can be administered every month to every 12 months, for example every month, every 2 months, every 3 months, every 4 months, every 5 months, every 6 months, every 7 months, every 8 months, every 9 months, every 10 months, every 11 months or every year. In some embodiments, the method further comprises administering an effective amount of DYRK1A inhibitor(s) (e.g. as a monotherapy). In some embodiments, the effective amount of DYRK1A inhibitor(s) can be administered orally, subcutaneously, intravenously. In some embodiments, the effective amount of DYRK1A inhibitor(s) (as a monotherapy) can be administered from one to four times a day, once a week, once every two weeks, once every three weeks or once a month. In some embodiments, the effective amount of DYRK1A inhibitor(s) (as a monotherapy) can be administered orally one to four times a day, once a week, once every two weeks, once every three weeks or once a month. In some embodiments, the effective amount of DYRK1A inhibitor(s) is administered orally from once to several times a day, for one week each month. In some embodiments, the effective amount of DYRK1A inhibitor(s) is administered orally from once to several times a day for two consecutive weeks each month. In some embodiments, the effective amount of DYRK1A inhibitor(s) is administered orally from once to several times a day on alternative weeks. In some embodiments, the effective amount of DYRK1A inhibitor(s) (as a monotherapy) is administered using an infusion pump. In some embodiments, the effective amount of DYRK1A inhibitor(s) is administered continuously for 7-14 days of each month using an infusion pump. In some embodiments, the effective amount of DYRK1A inhibitor(s) is administered by an infusion pump on alternative days. In some embodiments, the effective amount of DYRK1A inhibitor(s) is administered by an infusion pump 1, 2, 3, 4, 5 or 6 days each week.

In some embodiments, patients are monitored, 3 months after initiation of the combination therapy, for (1) the immune effects of the anti-CD3 antibodies (e.g. teplizumab) by, for example, assessment of the level of circulating exhausted T cells; and (2) beta cell regeneration by, for example, OGTT-stimulated C-peptide. Based on the results, a treatment algorithm can be implemented:
If the patient has more than 10% exhausted T cells (TIGIT+KLRG1+CD8+CD3+ cells) vs total CD3+ T cells, exhausted T cells are monitored annually and metabolic assessment is monitored at least quarterly. The level of exhausted T cells can be assessed for example by flow cytometry;
If the patient has less than 10% exhausted T cells vs CD3+ T cells, immunologic monitoring (e.g., by flow cytometry) can be every 6 months.

In some embodiments, when exhausted T cells return to baseline levels, the patient may receive a new course of anti-CD3 antibodies, and the monitoring cycle would start again with the 3-month exhausted T cell assessment. In some embodiments, the new course of anti-CD3 antibodies is a 10-day to 14-day course of teplizumab. In some embodiments, the new course of anti-CD3 antibodies is a 10-day or 12-day course of teplizumab. In some embodiments, the new course of anti-CD3 antibodies is a 12-day course of teplizumab. In some embodiments, the new course of anti-CD3 antibodies is a 14-day course of teplizumab.

In some embodiments anti-CD3 antibodies such as teplizumab can be re-dosed multiple times following the algorithm of the present disclosure or other algorithms or a calendar schedule.

In some embodiments, administration of DYRK1A inhibitor(s) can be discontinued after 3 months upon 2 consecutive quarterly metabolic assessments indicating stable regeneration of beta cell mass enabling freedom from exogenous insulin. Even moderate improvements in beta-cell mass can lead to a reduced requirement for exogenous insulin, improved glycemic control and a subsequent reduction in diabetic complications. The dosage of administered may then be reduced in strength and/or frequency or can be discontinued entirely if the patient continues to produce a normal amount of insulin without further treatment.

In some embodiments, the combination therapy may comprise co-administration of the active agents (anti-CD3 antibodies, DYRK1A inhibitors) during a treatment period and/or separate administration of single active agents during different time intervals in the treatment period.

In some embodiments, the methods of treatment of the disclosure result to at least a partial regeneration of pancreatic cells which contributes to an increase in beta-cell mass and to an improvement of a diabetic state. In some embodiments, administration of the combination therapy reverses the condition of diabetes partially or completely.

In some embodiments, anti-CD3 antibodies and DYRK1A inhibitor(s), or anti-CD3 antibodies and DYRK1A inhibitor(s) when used together can have additive or surprisingly synergistic effects on the treatment of T1D, such that the effect is greater than the effect that can be obtained with either compound alone.

Some aspects of the disclosure relates to a method of treating type 1 diabetes (T1D), comprising: administering to a subject in need thereof a 12-day course to 14-day course of anti-CD3 antibodies at a total dose of more than about 9000 μg/m², and administering to the subject an effective amount of a DYRK1A inhibitor(s). In some embodiments, the total dose of anti-CD3 antibodies is between about 9000 and about 9500 μg/m². In some embodiments, the total dose is between about 9000 and about 14000 μg/m².

In some embodiments, the 12-day course comprises a first dose of 106 μg/m² anti-CD3 antibodies on day 1, a second dose of 425 μg/m² anti-CD3 antibodies on day 2, and one dose of 850 μg/m² anti-CD3 antibodies on each of days 3-12, and wherein the total dose is approximately 9031 μg/m².

In some embodiments, the 12-day course comprises a first dose of 211 μg/m² anti-CD3 antibodies on day 1, a second dose of 423 μg/m² anti-CD3 antibodies on day 2, and one dose of 840 μg/m² anti-CD3 antibodies on each of days 3-12, and wherein the total dose is approximately 9034 μg/m².

In some embodiments, the method can include administering a first and a second 12-day courses of anti-CD3 antibodies. In some embodiments, the first and the second 12-day courses are administered at about 1-6 months, about 2-5 months or about 3 months interval.

In some embodiments, the method can include administering to the subject in need thereof a third or more 12-day course of anti-CD3 antibodies, each course at a total dose of more than about 9000 μg/m². In some embodiments, the method can include administering to the subject in need thereof a third or more 12-day course of anti-CD3 antibodies, each course at a total dose of between about 9030 and about 14000 μg/m².

In some embodiments, the third or more 12-day course of anti-CD3 antibodies comprises a first dose of 106 μg/m² anti-CD3 antibodies on day 1, a second dose of 425 μg/m² anti-CD3 antibodies on day 2, and one dose of 850 μg/m² anti-CD3 antibodies on each of days 3-12, and wherein the total dose of each course is approximately 9031 μg/m².

In some embodiments, the third or more 12-day course of anti-CD3 antibodies comprises a first dose of 211 μg/m² anti-CD3 antibodies on day 1, a second dose of 423 μg/m² anti-CD3 antibodies on day 2, and one dose of 840 μg/m² anti-CD3 antibodies on each of days 3-12, and wherein the total dose of each course is approximately 9034 μg/m².

In some embodiments, the third or more 12-day course of anti-CD3 antibodies is administered at about a 12 month to about a 24-month interval.

In some embodiments, the method can further include determining, about 3 months after the administration, a baseline of a level of TIGIT+KLRGT+CD8+ cells with respect to all CD3+ T cells, monitoring the level of the TIGIT+KLRGT+CD8+CD3+ T-cells and administering an additional 12-day course of anti-CD3 antibodies when the level of the TIGIT+KLRGT+CD8+CD3+ T-cells returns to the baseline level. In some embodiments, the determining of TIGIT+KLRGT+CD8+CD3+ T-cells is by flow cytometry. In some embodiments, if the subject has more than about 10% TIGIT+KLRGT+CD8+ T-cells in all CD3+ T cells, subsequent monitoring is annual. In some embodiments, if the subject has less than about 10% TIGIT+KLRGT+CD8+ T-cells in all CD8+ T cells, subsequent monitoring is every about 3-6 months.

In some embodiments, the administrating step results in reduction by at least 10% of exogenous insulin use, HbA1c levels, hypoglycemic episodes, or combinations thereof as compared to pre-treatment levels.

In some embodiments, each dose of anti-CD3 antibodies is administered parenterally.

In some embodiments, each dose of anti-CD3 antibodies is administered by intravenous infusion.

In some embodiments, the subject in need thereof is about 8 to 17 years old.

In some embodiments, the subject in need thereof is a non-diabetic subject who is at risk for T1D.

In some embodiments, the method comprises administering to the non-diabetic subject who is at risk for T1D a 14-day course IV infusion of anti-CD3 antibodies at about 60 μg/m², about 125 μg/m², about 250 μg/m², and about 500 μg/m², on days 1-4, respectively, and one dose of about 1000 μg/m² on each of days 5-14. In some embodiments, the cumulative dose is about 10935 μg/m².

In some embodiments, the method comprises administering to the non-diabetic subject who is at risk for T1D a 14-day course IV infusion of anti-CD3 antibodies at about 60 μg/m², about 125 μg/m², about 250 μg/m², and about 500 μg/m², on days 1-4, respectively, and one dose of about 1030 μg/m² on each of days 5-14. In some embodiments, the cumulative dose is about 11235 μg/m².

In some embodiments, the method comprises administering to the non-diabetic subject who is at risk for T1D a 14-day course IV infusion of anti-CD3 antibodies at about 100 μg/m², about 425 μg/m², about 850 μg/m², and about 850 μg/m², on days 1-4, respectively, and one dose of about 1000 μg/m² on each of days 5-14. In some embodiments, the cumulative dose is about 12225 μg/m².

In some embodiments, the method comprises administering to the non-diabetic subject who is at risk for T1D a 14-day course IV infusion of anti-CD3 antibodies at about 65 μg/m², about 125 μg/m², about 250 μg/m², and about 500 μg/m², on days 1-4, respectively, and one dose of about 1,070 μg/m² on each of days 5-14. In some embodiments, the cumulative dose is about 11640 μg/m².

In some embodiments, the effective amount of DYRK1A inhibitor(s) is administered orally, intraperitoneally, subcutaneously or by intravenous infusion.

In some embodiments, the subject in need thereof has a peak C-peptide level of ≥0.2 pmol/mL during a mixed meal tolerance test (MMTT).

In some embodiments, the subject receiving anti-CD3 antibodies has a higher mean C-peptide value after treatment, compared with a control receiving placebo.

In some embodiments, the method further includes assessing the area under the time-concentration curve (AUC) of C-peptide following a mixed meal tolerance test (MMTT), at 78 weeks.

In some embodiments, the subject in need thereof has at least 20% of beta-cell function prior the administration of the anti-CD3 antibodies and the DYRK1A inhibitor(s).

In some embodiments, the reduction of insulin use, HbA1c levels, hypoglycemic episodes, or combinations thereof is over a period of 12 months or more.

Aspects of the disclosure relate to methods of preventing or delaying onset of type 1 diabetes (T1D), comprising: administering prophylactically to a subject in need thereof a 14-day course of anti-CD3 antibodieat a total dose of about 9000 μg/m² to about 14000 μg/m² and administering to the subject in need thereof an effective amount of a DYRK1A inhibitor.

In some embodiments, the prophylactically effective amount of anti-CD3 antibodieis administered subcutaneously (SC) or intravenously (IV) or orally.

39 In some embodiments, the method comprises administering a 14 day course IV infusion at about 60 μg/m² on day 1, about 125 μg/m² on day 2, about 250 μg/m² on day 3, and about 500 μg/m² on day 4, and one dose of about 1000 μg/m² on each of days 5-14.

In some embodiments, the method comprises administering a 14 day course IV infusion at about 60 μg/m² on day 1, about 125 μg/m² on day 2, about 250 μg/m² on day 3 and about 500 μg/m² on day 4, respectively, and one dose of about 1030 μg/m² on each of days 5-14.

In some embodiments, the method comprises administering a 14 day course IV infusion at about 100 μg/m² on day 1, about 425 μg/m² on day 2, about 850 μg/m² on day 3, and about 850 μg/m² on day 4, and one dose of about 1000 μg/m² on each of days 5-14.

In some embodiments, the method comprises administering a 14 day course IV infusion at about 65 μg/m² on day 1, about 125 μg/m² on day 2, about 250 μg/m² on day 3, and about 500 μg/m² on day 4, and one dose of about 1070 μg/m² on each of days 5-14.

In some embodiments, the anti-CD3 antibody is teplizumab.

Provided herein is the use of an effective amount of anti-CD3 antibodies and an effective amount of DYRK1A inhibitor(s) for the treatment of type 1 diabetes in a subject in need thereof. In some embodiments, the ani-CD3 antibody is teplizumab.

### EXAMPLES

### Example 1. Teplizumab Population Pharmacokinetic Simulations

### Introduction

Teplizumab is a 150 kD monoclonal antibody that binds the CD3-ε epitope of the T cell receptor (TCR) complex. The primary mechanism of action of the antibody involves binding the CD3 antigen target on T cells. A population pharmacokinetic (PK) model that describes teplizumab concentrations following IV administration was developed. Teplizumab PK was described by a Quasi-Steady-State (QSS) approximation of the Target-Mediated Drug Disposition (TMDD) model. The aim of this investigation was to use the model to simulate and compare concentration-time profiles of teplizumab following several dosing regimens of interest.

### Objectives

The objectives of the analysis were:
To apply the previously developed population PK model to simulate the following three dosing regimens:
   “Herold Dosing Regimen”: Day 1: 51 μg/m²; Day 2: 103 μg/m²; Day 3: 207 μg/m²; Day 4: 413 μg/m²; Days 5-14: 826 μg/m²;
   Regimen 1: Day 1: 211 μg/m²; Day 2: 423 μg/m²; Days 3-12: 840 μg/m²;
   Regimen 2: Day 1: 106 μg/m²; Day 2: 425 μg/m²; Day 3-12: 850 μg/m².
To illustrate and compare concentration-time courses of teplizumab for the 3 dosing regimens listed above.

### Subjects and Methods

### Dosing Regimens

The Herold regimen is a 14-day course of teplizumab consisting of daily intravenous (IV) infusions (over at least 30 minutes) of 51 μg/m², 103 μg/m², 207 μg/m², and 413 μg/m² on Study Days 1-4, respectively, and an infusion of 826 μg/m² on each of Study Days 5-14. The total dose for a 14-day course is approximately 9034 μg/m². For subjects with body surface area (BSA) of 1.92 m², this dosing schedule delivers approximately 17 mg of teplizumab. The maximum amount of drug delivered at steady-state was designed to provide coating of 50% to 80% of the available CD3 on T cells, with no large excesses of free, unbound drug (projected to be <200 ng/mL at steady-state).

The new Regimen 1 is a 12-day course of teplizumab consisting of daily IV infusion (over at least 30 minutes) of 211 μg/m² and 423 μg/m² on Study Days 1 and 2, respectively, and an infusion of 840 μg/m² on each of Study Days 3-12. The total dose for a 12-day course is approximately 9034 μg/m².

The new Regimen 2 is a 12-day course of teplizumab consisting of daily IV infusion (over at least 30 minutes) of 106 μg/m² and 425 μg/m² on Study Days 1 and 2, respectively, and an infusion of 850 μg/m² on each of Study Days 3-12. The total dose for a 12-day course is approximately 9031 μg/m².

As evident, the same total dose is to be delivered by all three regimens, but in Regimens 1 and 2, delivery is over 12 days rather than 14 days of the original Herold regimen.

### Simulations

The final model of the previous analysis was used for simulations. Concentration-time courses were simulated for 40 days (Day 0 to Day 40), with 10 time points each day. The model included the study effect as patients from Protégé Encore study were found to have higher clearance and central volume than patients from Protégé study. Therefore, the simulations were conducted separately for these 2 studies. Covariate values of four typical patients were used for simulations, specifically:
Adult patients with no detected anti-drug antibodies [ADA]: 18 years old, 60 kg males with BSA of 1.67 m²;
Adult patients with high level of ADAs: 18 years old, 60 kg males with BSA of 1.67 m²;
Pediatric patients with no detected ADA: 13 years old, 45 kg males with BSA of 1.33 m²;
Pediatric patients with high level of ADAs: 13 years old, 45 kg males with BSA of 1.33 m²

For each of these patients, population predictions of concentrations over time were computed for each of 3 dosing regimens, and were then compared graphically. Then, the parameters of 1000 similar patients were simulated using the model-estimated inter-individual variability, and individual concentration-time courses were computed using the model. Median and 90% prediction intervals of simulated concentrations at each time point were computed for each regimen, and were compared graphically. In addition, mean and standard deviations of the simulated values at 1 day after the last dose were computed and compared.

### Software

The simulations were conducted using the NONMEM software, Version 7.4.1 (ICON Development Solutions). Computer resources included personal computers with Intel® processors, Windows 7 Professional or later operating system and Intel® Visual Fortran Professional Compiler (Version 11.0). Graphical and all other statistical analyses, including evaluation of NONMEM outputs were performed using R version 3.4.4 for Windows (R project, www.r-project.org/).

### Results

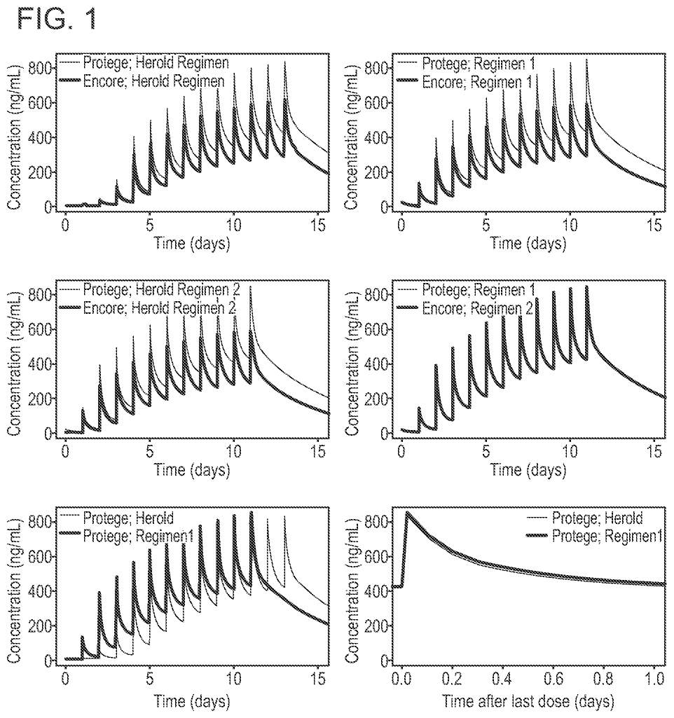

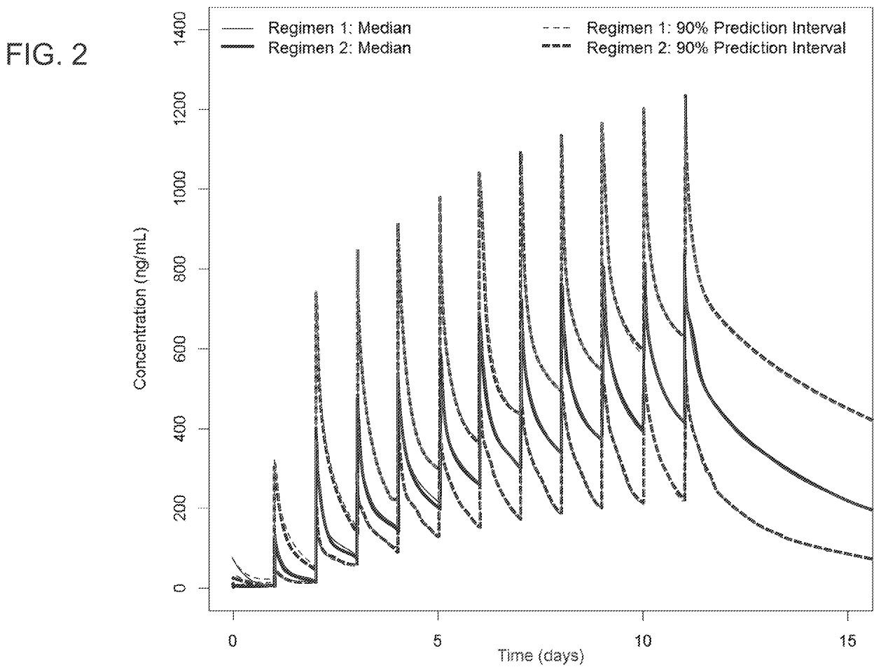

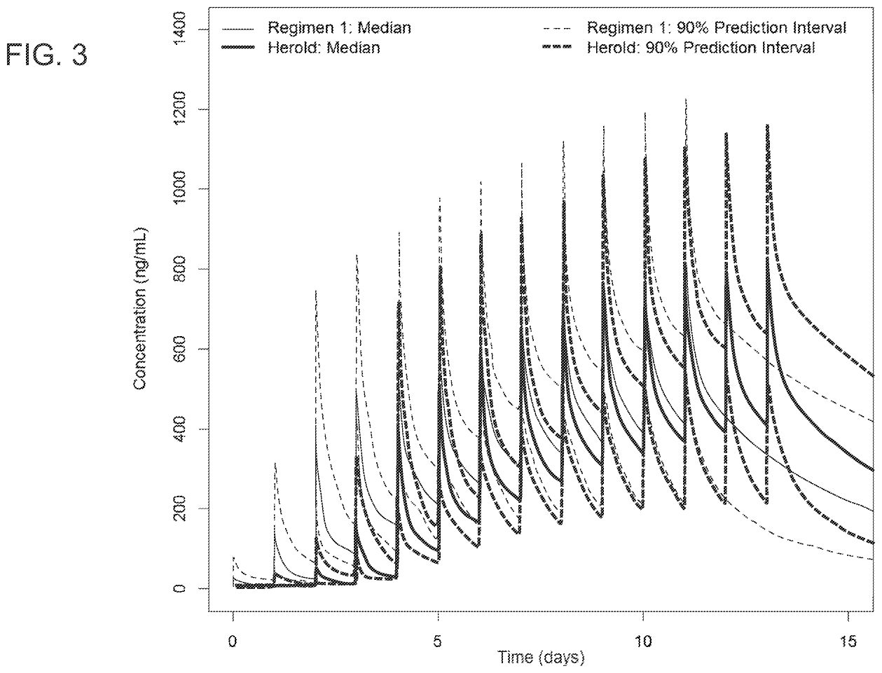

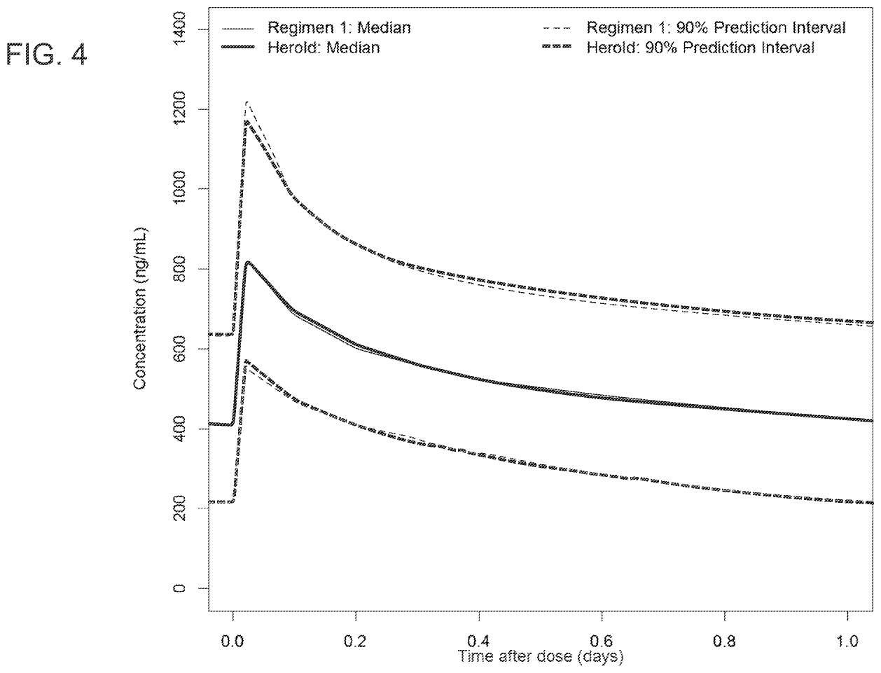

The results of the simulations for typical adult patients with no detected ADAs are shown in FIG. 1. Concentrations in the Protégé study were predicted to be higher than in the Encore study for all dosing regimens. The concentrations in Dosing Regimens 1 and 2 were nearly indistinguishable except for minor differences during the first two days of dosing. During the first 12 days of dosing, concentrations in the Herold dosing regimen were lower compared to Dosing regimens 1 and 2, but they were nearly identical following the last dose (on Day 14 for the Herold regimen, and Day 12 for Regimens 1 and 2). The simulations that included inter-individual variability (FIGS. 2-4, Table 1) confirmed these observations.

TABLE 1
Teplizumab Concentration Predictions: C_{trough} 1 day After the Last Dose Table
illustrates mean and standard deviation of predicted concentrations (ng/mL)
over 1000 simulated subjects from Protégé study

| | | | Mean (standard deviation) | | |
|---|---|---|---|---|---|
| | | | Excludes | | Includes |
| | Dosing | Time | residual | | residual |
| Patient Population | Regimen | Point | error | | error |
| Age = 18 years, WT = 60 | Herold regimen | 14 days | 425 | (130) | 426 (220) |
| kg, BSA = 1.67 m² male | Regimen 1 | 12 days | 432 | (133) | 432 (224) |
| subjects with no ADA | Regimen 2 | 12 days | 435 | (134) | 435 (225) |
| detected | | | | | |
| Age = 18 years, WT = 60 | Herold regimen | 14 days | 184 | (82) | 183 (113) |
| kg, BSA = 1.67 m² male | Regimen 1 | 12 days | 189 | (84) | 197 (123) |
| subjects with extremely | Regimen 2 | 12 days | 191 | (85) | 199 (124) |
| high ADA levels | | | | | |
| (HAHA2 = 10) | | | | | |
| Age = 13 years, WT = 45 | Herold regimen | 14 days | 394 | (120) | 393 (206) |
| kg, BSA = 1.33 m² male | Regimen 1 | 12 days | 410 | (123) | 403 (210) |
| subjects with no ADA | Regimen 2 | 12 days | 413 | (123) | 406 (211) |
| detected | | | | | |
| Age = 13 years, WT = 45 | Herold regimen | 14 days | 171 | (79) | 174 (112) |
| kg, BSA = 1.33 m² male | Regimen 1 | 12 days | 173 | (77) | 173 (107) |
| subjects with extremely | Regimen 2 | 12 days | 175 | (78) | 175 (108) |
| high ADA levels | | | | | |
| (HAHA2 = 10) | | | | | |

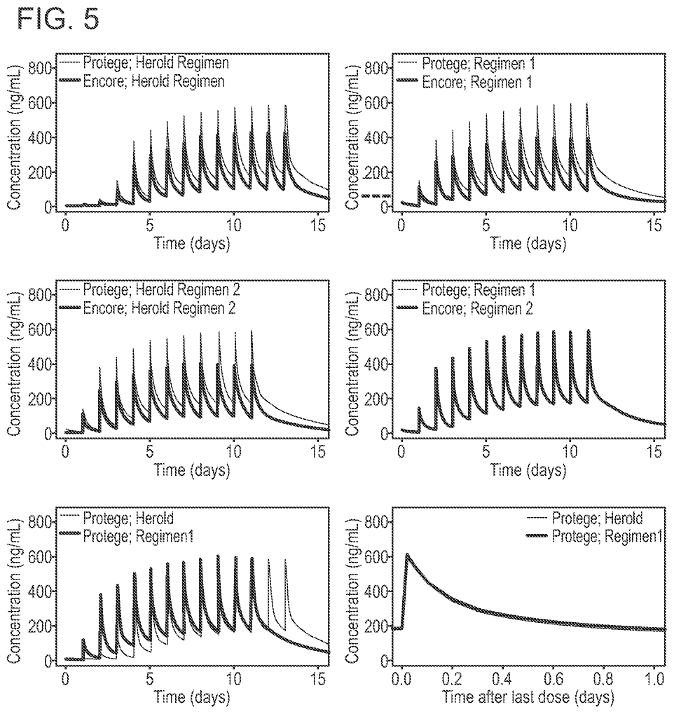

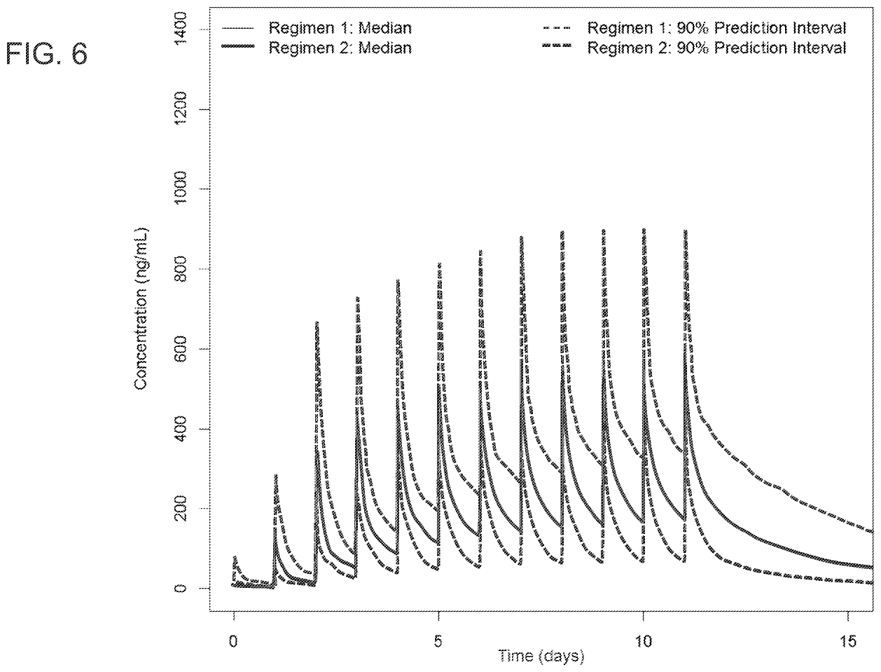

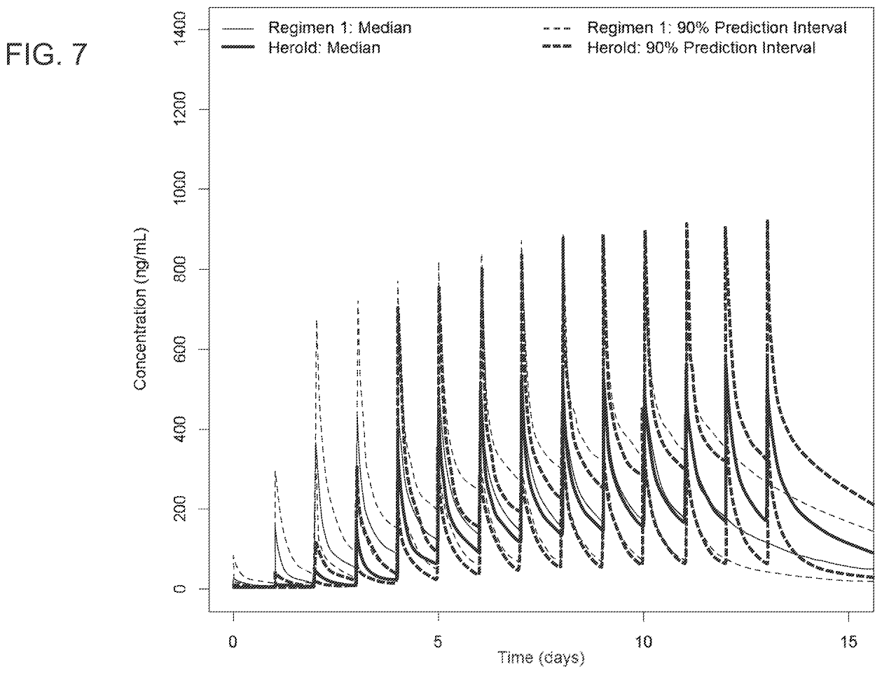

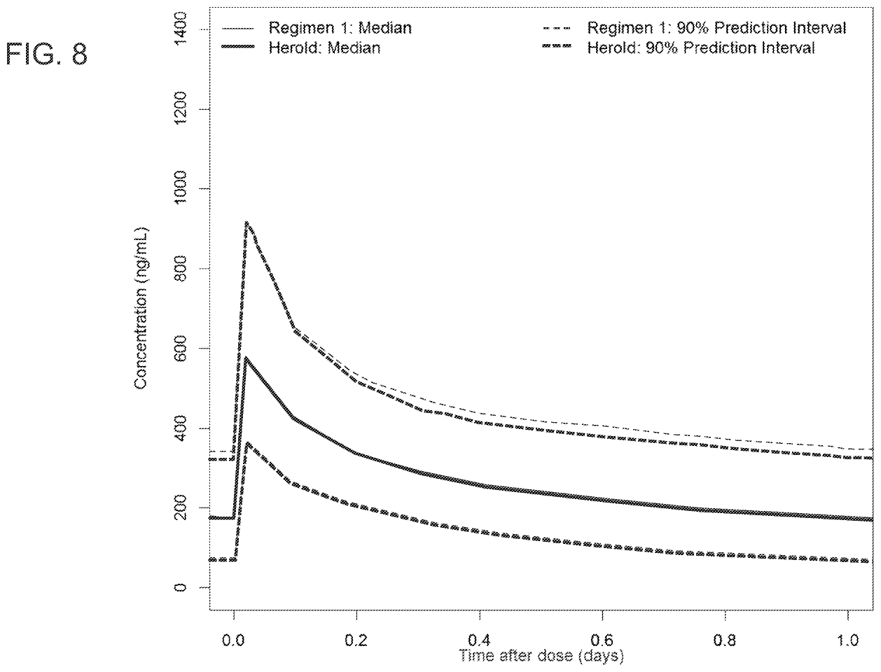

The results of the simulations for typical adult patients with high level of detected ADAs are shown in FIGS. 5-8. As expected, overall teplizumab levels are much lower for subjects with very high immunogenic response, but the conclusions about differences between the three investigated dosing regimens still hold.

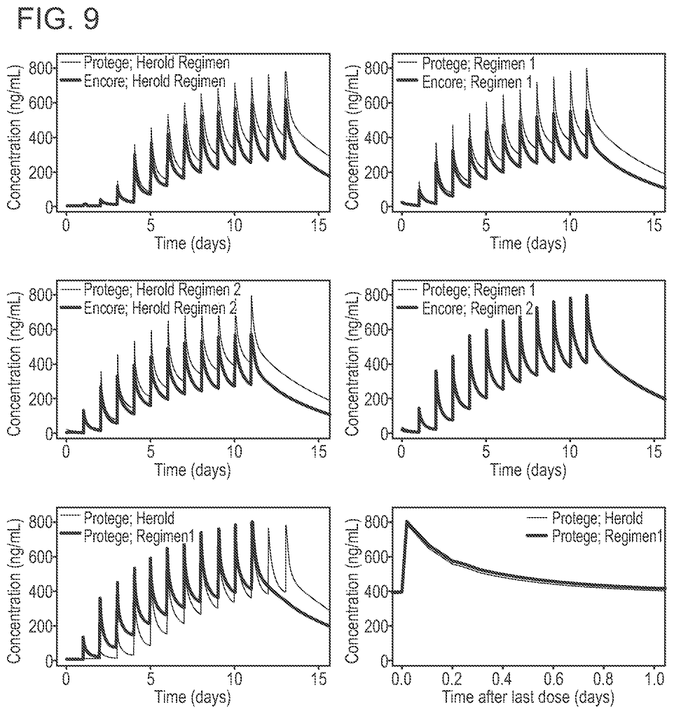

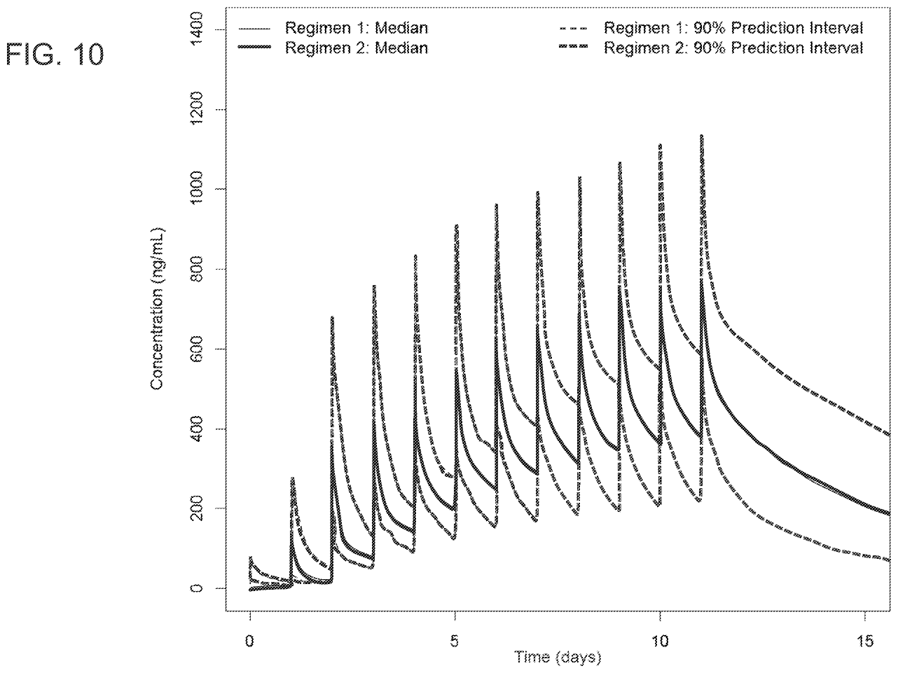

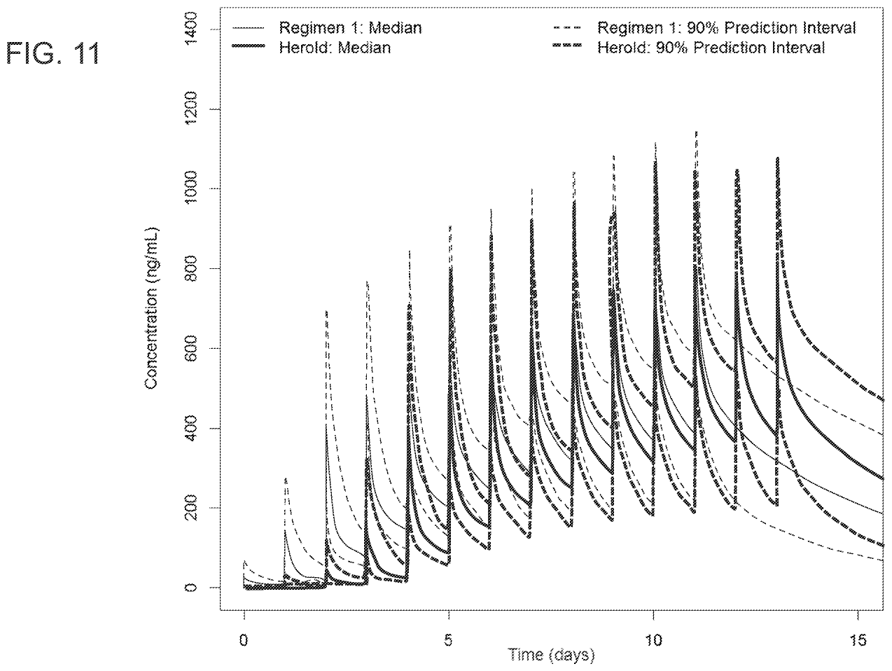

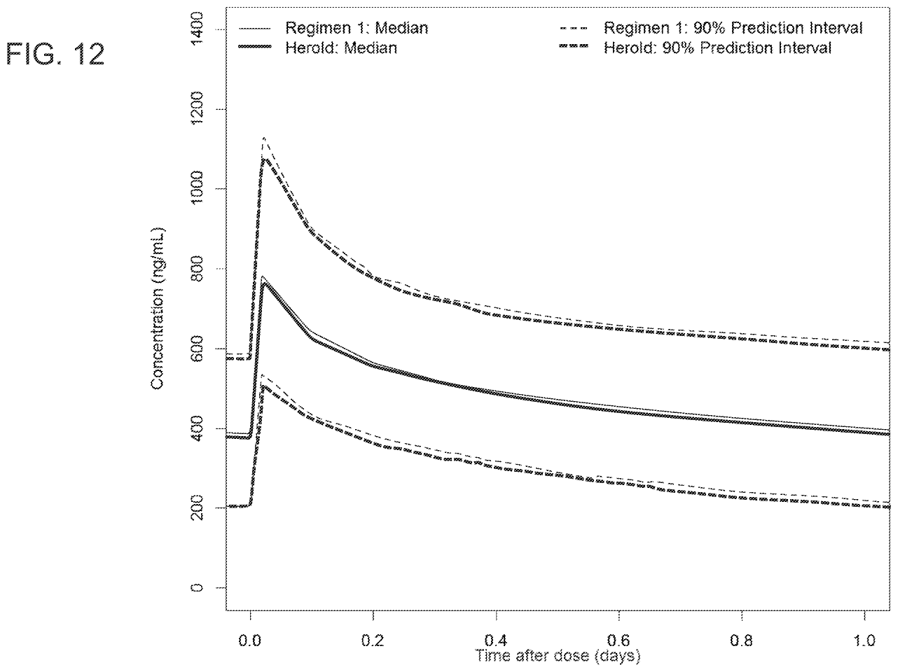

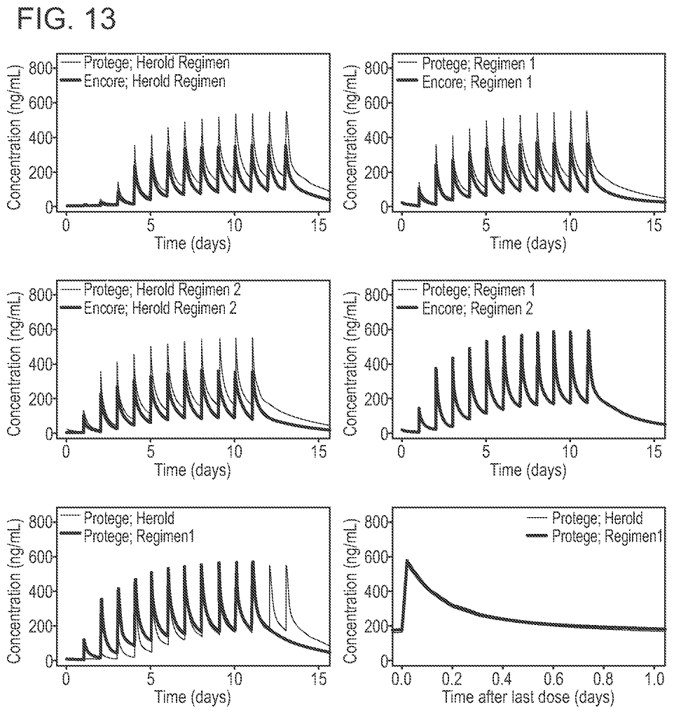

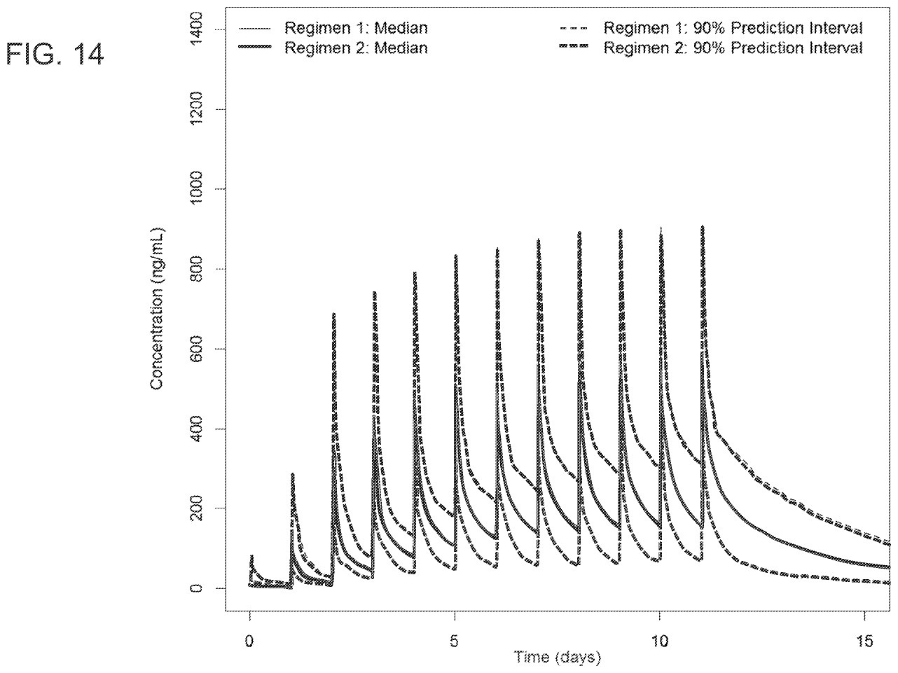

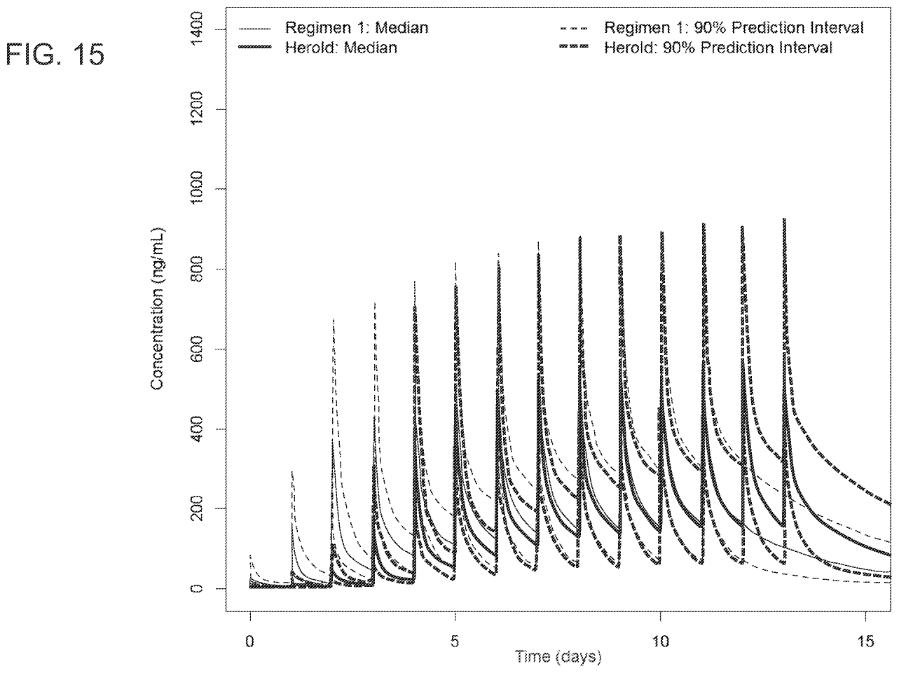

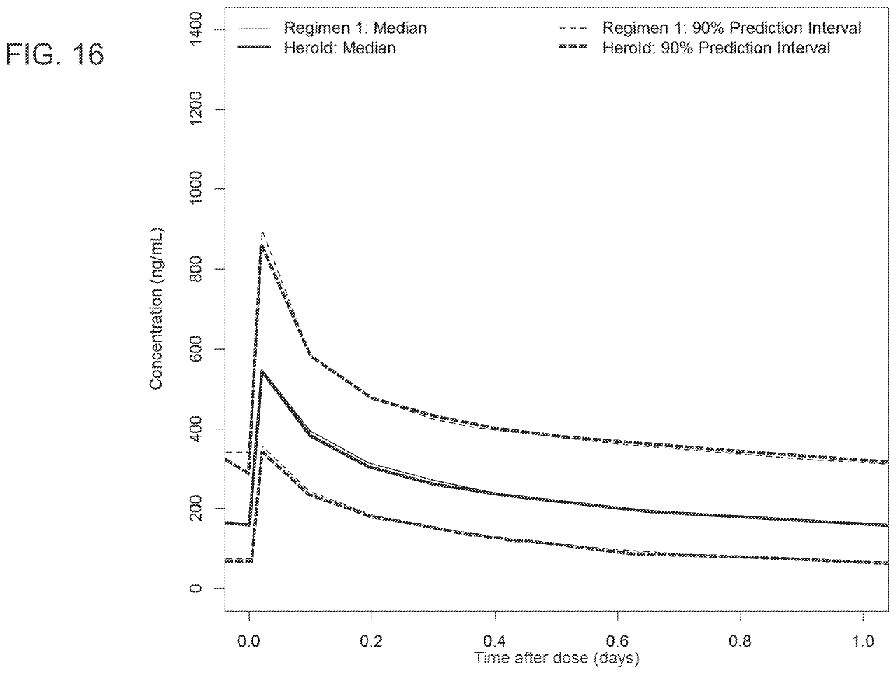

The results of the simulations for typical pediatric patients are shown in FIGS. 9-16. They are very similar to those for adult patients, indicating the BSA-proportional dosing provides similar exposure for pediatric and adult populations.

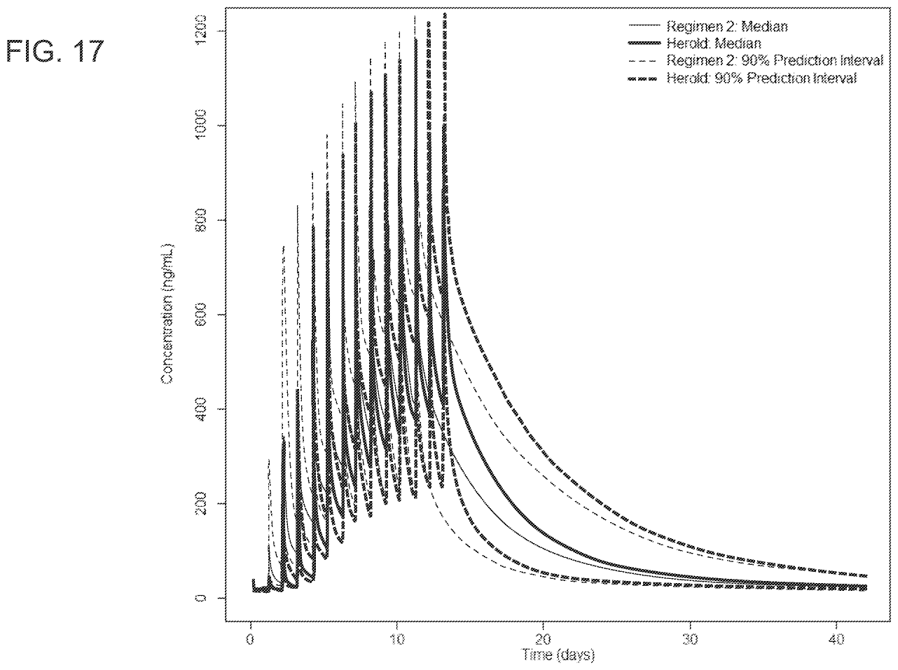

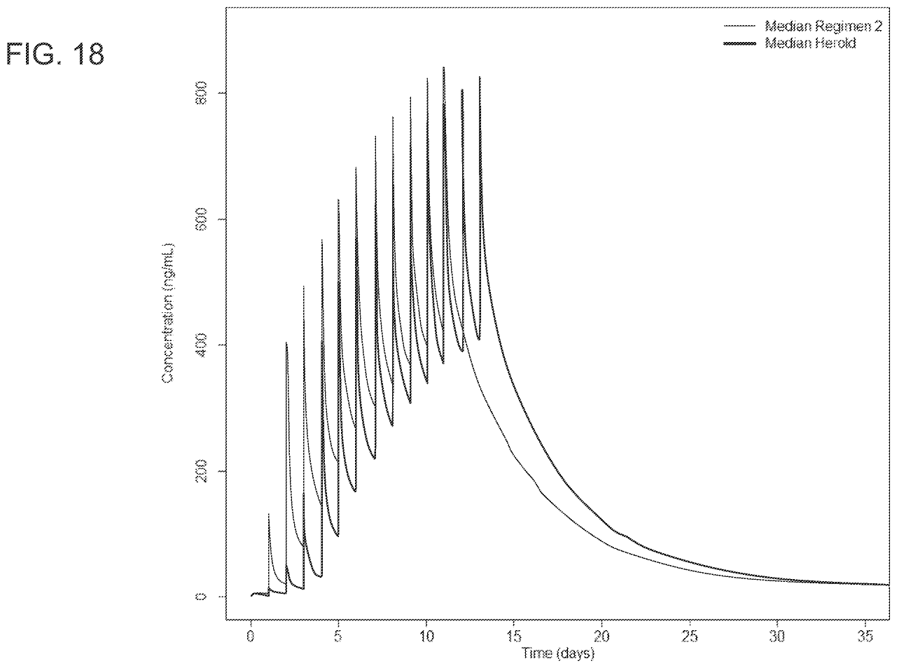

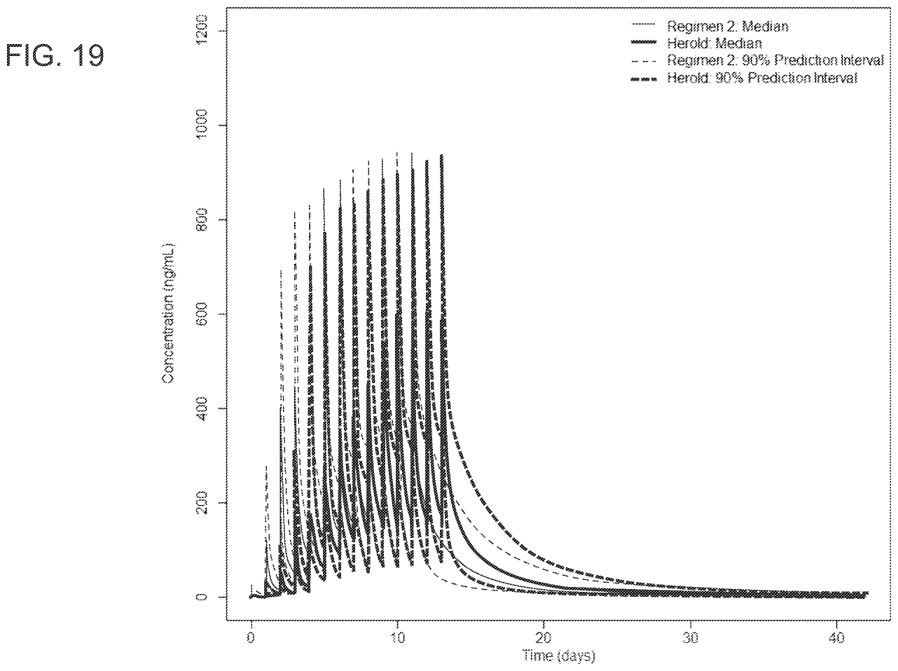

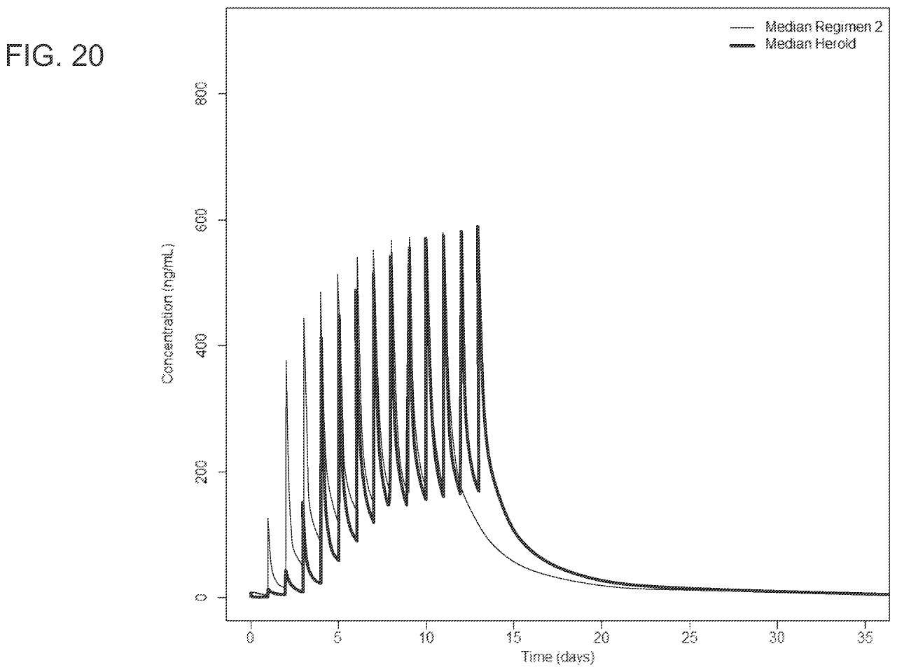

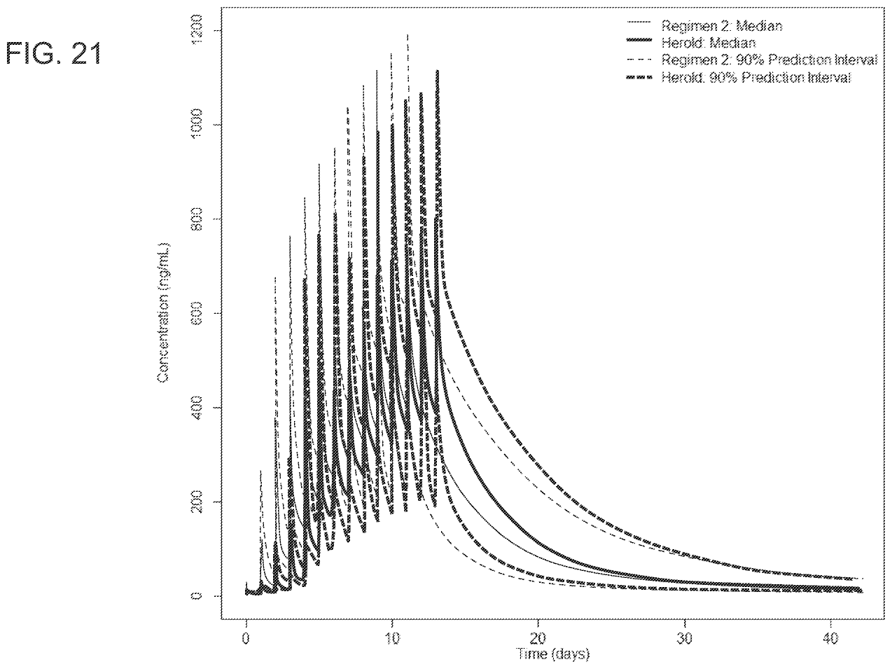

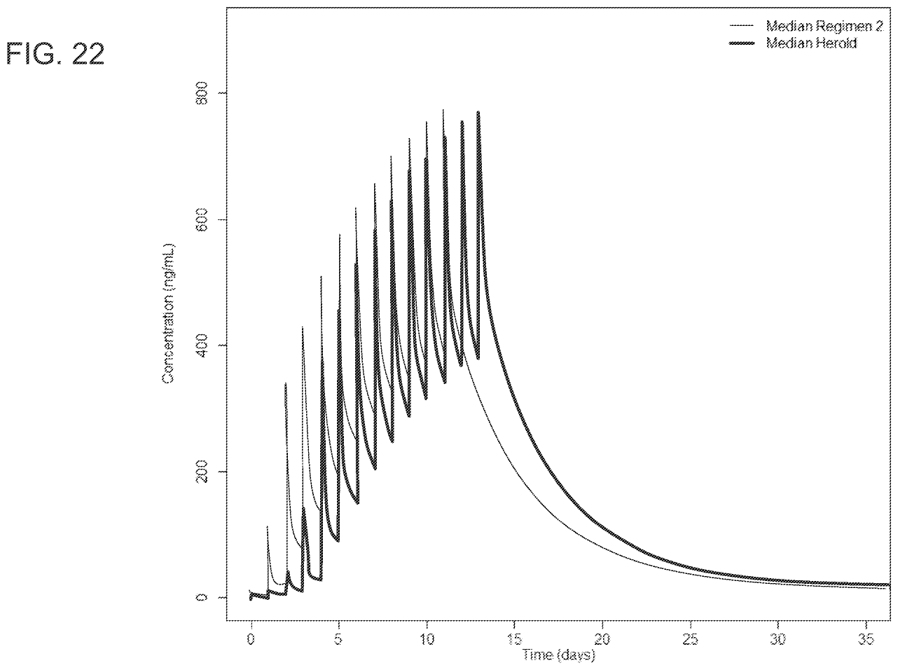

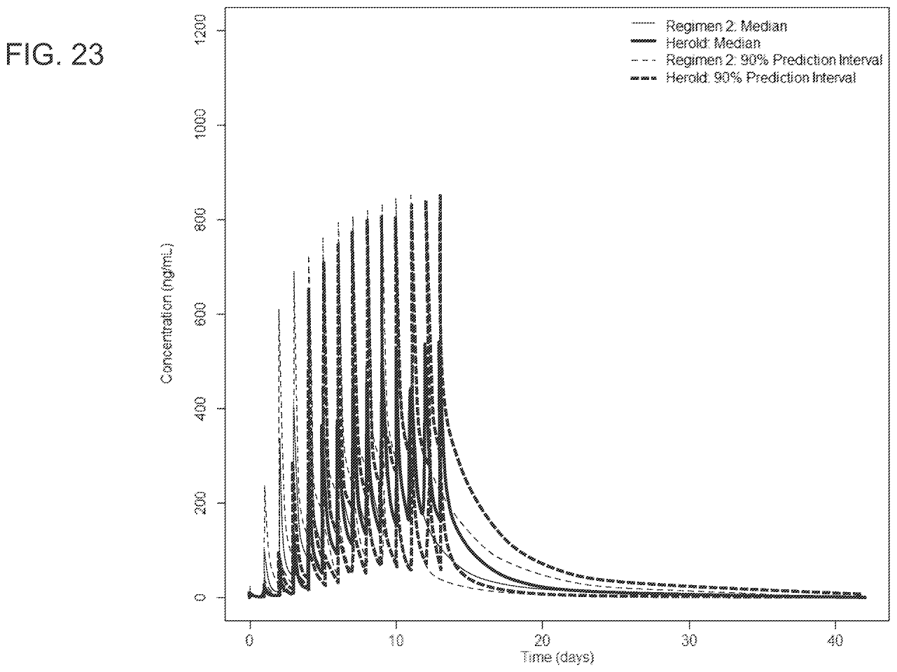

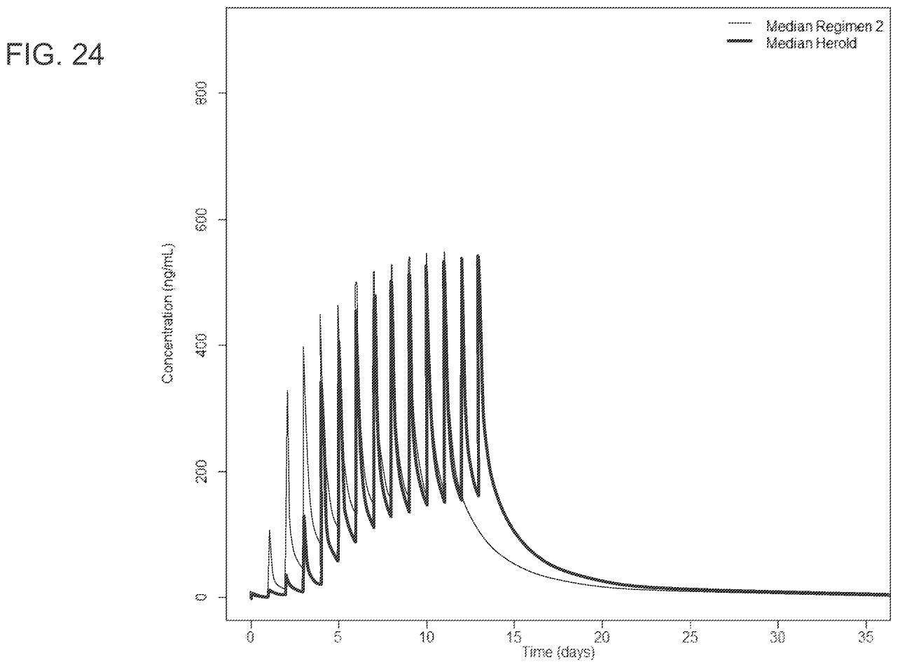

FIGS. 17-24 show concentration profiles comparing Herold Regimen and Regimen 2 for a longer time period and Table 2-Table 3 summarized Cmax and AUC from 0 to 42 days in the simulations. Figures show that by day 42 concentrations are very low, so values for AUC₀₋₄₂ are essentially the same as for AUCinfinity.

TABLE 2
Teplizumab Concentration Predictions: Cₘₐₓ Table illustrates mean and
standard deviation of predicted maximum concentrations (ng/mL) over
1000 simulated subjects using Protégé Model 205

| | | Mean (standard deviation) | |
|---|---|---|---|
| | | Excludes | Includes |
| | Dosing | residual | residual |
| Patient Population | Regimen | error | error |
| Age = 18 years, WT = 60 | Herold regimen | 849 (205) | 850 (405) |
| kg, BSA = 1.67 m² male | Regimen 1 | 855 (200) | 856 (399) |
| subjects with no ADA | Regimen 2 | 863 (202) | 864 (402) |
| detected | | | |
| Age = 18 years, WT = 60 | Herold regimen | 609 (178) | 612 (304) |
| kg, BSA = 1.67 m² male | Regimen 1 | 607 (175) | 610 (318) |
| subjects with extremely | Regimen 2 | 614 (177) | 617 (321) |
| high ADA levels | | | |
| (HAHA2 = 10) | | | |
| Age = 13 years, WT = 45 | Herold regimen | 788 (189) | 785 (377) |
| kg, BSA = 1.33 m² male | Regimen 1 | 792 (199) | 798 (386) |
| subjects with no ADA | Regimen 2 | 799 (200) | 806 (389) |
| detected | | | |
| Age = 13 years, WT = 45 | Herold regimen | 559 (159) | 566 (292) |
| kg, BSA = 1.33 m² male | Regimen 1 | 561 (151) | 552 (271) |
| subjects with extremely | Regimen 2 | 568 (153) | 558 (274) |
| high ADA levels | | | |
| (HAHA2 = 10) | | | |

TABLE 3
Teplizumab Concentration Predictions: AUC₀₋₄₂ Table illustrates
mean and standard deviation of predicted AUC from 0
to 42 days (ng/ml*day) over 1000 simulated subjects
using Protégé Model 205

| | | AUC₀₋₄₂ Mean | |
|---|---|---|---|
| | | standard | |
| Patient Population | Dosing Regimen | (deviation) | |
| Age = 18 years, WT = 60 | Herold regimen | 6548 | (819) |
| kg, BSA = 1.67 m² male | Regimen 1 | 6662 | (2085) |
| subjects with no ADA | Regimen 2 | 6659 | (2084) |
| detected | | | |
| Age = 18 years, WT = 60 | Herold regimen | 3082 | (630) |
| kg, BSA = 1.67 m² male | Regimen 1 | 3099 | (1044) |
| subjects with extremely | Regimen 2 | 3098 | (1044) |
| high ADA levels | | | |
| (HAHA2 = 10) | | | |
| Age = 13 years, WT = 45 | Herold regimen | 5939 | (750) |
| kg, BSA = 1.33 m² male | Regimen 1 | 6032 | (1936) |
| subjects with no ADA | Regimen 2 | 6029 | (1935) |
| detected | | | |
| Age = 13 years, WT = 45 | Herold regimen | 2830 | (557) |
| kg, BSA = 1.33 m² male | Regimen 1 | 2837 | (902) |
| subjects with extremely | Regimen 2 | 2836 | (902) |
| high ADA levels | | | |
| (HAHA2 = 10) | | | |

### CONCLUSIONS

The simulations indicated that:
Predicted concentrations of teplizumab are nearly identical for 2 suggested dosing regimens (Regimen 1 and Regimen 2) except for the first day of dosing;
Predicted concentrations of teplizumab increase faster during dosing for Regimens 1 and 2 compared to Herold regimen, but they are nearly identical for all regimens at the last day of dosing;
Predicted concentrations of teplizumab at 1 day after the last dose are nearly identical for all 3 regimens;
BSA-proportional dosing provides homogeneous exposure levels for adult and pediatric subjects with different body size measures.

### Example 2. A Phase 3, Randomized, Double-Blind, Multinational, Placebo-Controlled Study to Evaluate Efficacy and Safety of Teplizumab (PRV-031), a Humanized, FcR NonBinding, Anti-CD3 Monoclonal Antibody, in Children and Adolescents with Newly Diagnosed Type 1 Diabetes (T1D)

Teplizumab (also known as PRV-031, hOKT3γ1 [Ala-Ala], and MGA031) is a humanized 150-kilodalton monoclonal antibody (mAb) that binds to the CD3-ε epitope of the T cell receptor. Teplizumab was developed when preclinical studies demonstrated that targeting T cells (the cells that are instrumental in initiating and coordinating the autoimmune process responsible for type 1 diabetes [T1D] mellitus) via this mechanism altered diabetes immunopathogenesis and prevented and reversed disease in relevant animal models. The goal of this study is to evaluate teplizumab in children and adolescents very recently diagnosed with T1D. Teplizumab holds the promise to be the first disease modifying therapy available to improve both the medical management and overall outlook in those who suffer the most devastating short- and long-term consequences of this disease.

### Hypothesis

The hypothesis of this study is that teplizumab is safe, well-tolerated, and effective in slowing the loss of β cells and maintaining a clinically relevant level of β cell function in children and adolescents newly diagnosed with T1D while improving key aspects of T1D clinical management over an 18-month period.

### Objectives

The primary objective is:
To determine whether two courses of teplizumab administered 6 months apart slow the loss of β cells and preserve β cell function over 18 months (78 weeks) in children and adolescents 8-17 years old who have been diagnosed with T1D in the previous 6 weeks.

The secondary objectives are:
To evaluate participant improvements in key clinical parameters of diabetes management, including insulin use, glycemic control (including hemoglobin Alc [HbA1c] and time in glycemic target range [TIR]), and clinically important hypoglycemic episodes
To determine the safety and tolerability of two courses of teplizumab, administered intravenously (IV) 6 months apart
To evaluate the pharmacokinetics (PK) and immunogenicity of two courses of IV teplizumab

The exploratory objectives are:
To assess β cell function and T1D-focused clinical parameters
To evaluate immunologic, endocrinologic, molecular, and genetic markers

### Endpoints

1. The primary endpoint is:
   The area under the time-concentration curve (AUC) of C-peptide after a 4-hour (4 h) mixed meal tolerance test (MMTT), a measure of endogenous insulin production and R cell function, at Week 78.
2. The secondary endpoints are as follows:
A. Key Clinical Endpoints:
   Exogenous insulin use: defined as a daily average in units per kilogram per day (U/kg/d), at Week 78
   HbA1c levels: expressed in % and mmol/mol, at Week 78
   TIR: expressed as a daily average of the percentage of time in a 24-hour day a participant's blood glucose (BG) is >70 but≤180 mg/dL (>3.9 to ≤10.0 mmol/L), assessed using continuous glucose monitoring (CGM), at Week 78
   Clinically important hypoglycemic episodes: defined as the total number of episodes of a BG reading of <54 mg/dL (3.0 mmol/L) and/or episodes of severe cognitive impairment requiring external assistance for recovery, from randomization through Week 78
B. Safety Endpoints:
   Incidence of treatment-emergent adverse events (TEAEs), adverse events of special interest (AESIs), and serious adverse events (SAEs)
   Incidence of treatment-emergent infections of special interest, including but not limited to tuberculosis, an infection requiring IV antimicrobial treatment or hospitalization, Epstein-Barr virus (EBV) and cytomegalovirus (CMV) infection, or significant viremia (ie, DNA-based polymerase chain reaction viral load >10,000 copies per mL or 10⁶ cells), and herpes zoster
   Incidence and severity of immediate or delayed study drug infusion-related reactions, such as hypersensitivity reactions, pain requiring interruption or discontinuation of infusions, cytokine release syndrome, and serum sickness
C. PK and Immunogenicity Endpoints:
   Teplizumab serum concentrations
   Incidence and titers of anti-teplizumab antibodies after treatment courses
3. The exploratory endpoints are as follows:
A. Assessments of β cell function and health throughout the study:
   4 h MMTT C-peptide AUC
   Participants with the recognized clinically significant stimulated peak C-peptide of ≥0.2 pmol/mL during 4 h and 2-hour (2 h) MMTTs
   Proinsulin-to-C-peptide ratios, a measure of β cell endoplasmic reticulum stress and dysfunction
B. T1D-focused Clinical Endpoints during the study unless otherwise noted:
   Exogenous insulin use (in U/kg/day)
   HbA1c levels
   Participants with poor glycemic control, defined as HbA1c of ≥9%
   The number of participants who do not require exogenous insulin because they are able to achieve local, regional, or national age-based glycemic management goals for HbA1c and/or routine blood glucose levels
   Evaluations of glycemic control based on BG values obtained from intermittent (ie, spot-check, fingerstick) glucometer readings
   Evaluations of glycemic control based on BG values obtained from CGM readings, including but not limited to TIR; time in hyperglycemia and hypoglycemia ranges; daily, daytime, and nighttime average BG levels and estimated HbA1c; and glycemic variability
   Clinically important hypoglycemic episodes from randomization through Week 39 and from Week 39 through Week 78
   Incidence of “typical” hypoglycemia, defined as BG levels ≥54 mg/dL (3.0 mmol/L) but <70 mg/dL (3.9 mmol/L) and/or non-severe clinical episodes
   Incidence of diabetic ketoacidosis (DKA) requiring medical attention, defined as a hyperglycemic episode with serum or urine ketones elevated beyond upper limit of normal (ULN) along with serum bicarbonate <15 mmol/L or blood pH<7.3, or both, and resulting in outpatient, emergency room visit or hospitalization
   Patient-reported outcomes measured by instruments, such as Quality of Life Inventory™ (PedsQL) Diabetes Module, the Hypoglycemia Fear Scale (HFS), and the Diabetes Treatment Satisfaction Questionnaire (DTSQ)
   Impact on family life, measured by the parent-reported PedsQL Family Impact questionnaire
C. Composite Clinical Endpoints:
   Participants with both HbA1c in the American Diabetic Association (ADA) target range (ie, <7.5%) and exogenous insulin dose in specific ranges (<0.25, 0.25 to <0.50, 0.50 to <0.75, 0.75 to <1.0, 1.0 to <1.25, and ≥1.25 U/k/d)
   Participants with both HbA1c of <6.5% and <7.0% and exogenous insulin dose of <0.5 U/kg/day or 0.25 U/kg/day
D. Other Endpoints during the study:
   Number, type, and titer of T1D autoantibodies
   Association of human leukocyte antigen (HLA) type with clinical, metabolic and immune assessments Overview of Study Design

This is a Phase 3, randomized, double-blind, placebo-controlled, multinational, multicenter study. Approximately 300 participants are enrolled and randomly assigned at a ratio of 2:1 to either the teplizumab group (N=200) or the placebo group (N=100).

To minimize bias in treatment assignment, potential confounders, and enhance the validity of statistical analysis, participants are randomized at a 2:1 ratio using randomly permuted blocks and stratification based on the following criteria:
Peak C-peptide level at screening: within the range of 0.2 (inclusion criterion) to 0.7 pmol/mL (inclusive) versus >0.7 pmol/mL
Age at randomization: within the range of 8 to 12 years (inclusive) versus >12 to 17 years

Teplizumab or matching placebo are administered via IV infusion in two courses, with the first course starting on Day 1 (Week 1) and the second course approximately 6 months later at Day 182 (Week 26). Each course of treatment include daily infusions for 12 days.

The total study duration for each participant is up to 84 weeks. This includes a screening period of up to 6 weeks and a post-randomization period of 78 weeks. The treatment period includes two 12-day treatment courses separated by 6 months and a post-treatment observation period of approximately 52 weeks.

### Study Population

This study enrolls male and female participants 8 to 17 years of age with new-onset T1D who are able to be randomized and initiate study treatment within 6 weeks of their diagnosis. To be eligible for randomization, participants must be positive for at least one T1D-associated autoantibody and have a peak stimulated C-peptide of ≥0.2 pmol/mL at screening. They must also meet all of the specific inclusion criteria and none of the exclusion criteria.

### Dosage and Administration

On the day of randomization (Day 1), each participant receives the first dose of the study drug in the first 12-day treatment course, as shown in the table below. On approximately Day 182, each participant receives the first dose of the second 12-day course. The study drugs (teplizumab or placebo) are administered via IV infusion at the study site or other qualified facility by study-approved personnel. The doses of study drug is calculated based on the participant's body surface area (BSA) measured on the first day of each treatment course. No dose adjustment is permitted.

| Treatment | | |
|---|---|---|
| name | Teplizumab | Placebo |
| Description | Sterile solution for | Sterile solution for |
| | injection | injection |
| Doses in | Day 1: 106 μg/m² | Matching volumes to |
| each course | Day 2: 425 μg/m² | active drug |
| | Days 3-12: 850 μg/m² | |
| | Total per course: 9.0 mg/m² | |
| Frequency | Two courses starting | Two courses starting |
| | at Week 1 and Week 26 | at Week 1 and Week 26 |
| Delivery | IV infusion | IV infusion |
| method | | |

### Key Evaluations

MMTT: In order to quantitate endogenous β cell function, participants undergo standardized provocative metabolic testing for C-peptide (a 1:1 by-product of insulin production). Participants consume a fixed amount of a beverage with known amounts of carbohydrates, fats, and protein. Following consumption, BG, insulin, and C-peptide levels are measured over time. A 2 h MMTT is conducted at screening, and 4 h MMTTs is conducted at randomization and Weeks 26, 52, and 78 for key endpoint assessments.

HbA1c: This is the percent of red blood cells (measured as hemoglobin) that has become non-enzymatic glycated proportional to blood glucose levels. This indicates, on average, approximately a 3-month average of blood glucose values. It is a key clinical target in the management of T1D.

Insulin use: As an average over 7 days of data collected before each specified visit to quantify exogenously injected insulin.

Hypoglycemia: Clinically important and potentially life-threating hypoglycemia is the result of insulin therapy and more likely to occur in patients who are attempting to achieve glycemic control goals. This study ask participants to record information regarding BG levels of <70 mg/dL (3.9 mmol/L) and/or events that are consistent with hypoglycemia. A particular focus is on clinically significant hypoglycemic events that are defined as a reliable glucose reading of <54 mg/dL (3.0 mmol/L) and/or severe cognitive impairment and/or physical status requiring external assistance for recovery.

Glucose Monitoring: Intermittent glucose monitoring (e.g, spot-check or fingerstick) performed by participants or caregivers multiple times a day as a necessary part of glycemic management to gauge insulin dosing and assist in diet and activity. All participants are to bring in their glucometers at all visits for review. In addition to data regarding glycemic control, at specified times during the study, participants report their daily before-meal and before-bedtime BG readings and have glucose levels assessed for 2-week intervals using CGM.

Quality of Life Questionnaires: Surveys is used to assess the general health and wellbeing of participants and the effects of teplizumab, such as the PedsQL Diabetes Module, HFS, DTSQ, and parent-reported PedsQL Family Impact Module.

Pharmacokinetic and Immunogenicity Evaluations: Teplizumab concentrations are analyzed in blood samples collected at specified time points throughout the study. Anti-teplizumab antibodies are determined, including those that are neutralizing antibodies (NAbs).

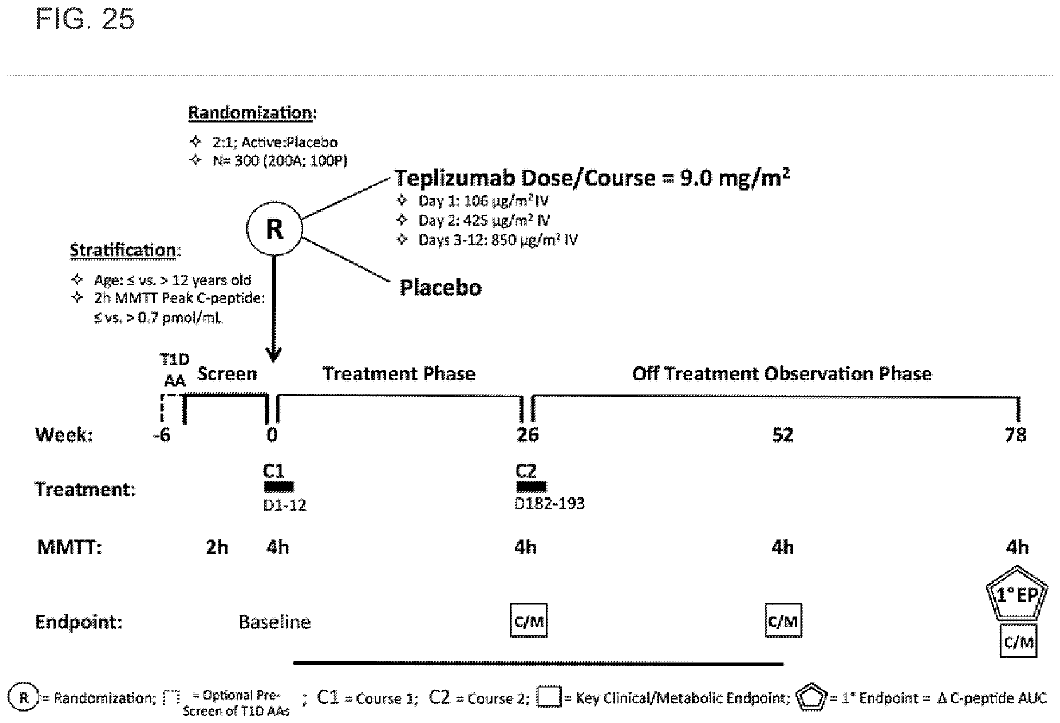

A diagram of the study design is provided in FIG. 25.

The study focuses on individuals who have a significant amount of β cell functional capacity. It is recognized that β cells continue to be lost following T1D diagnosis. To maximize the effect of β cell preservation in patients with a recoverable level endogenous insulin production, this study recruits participants within 6 weeks from T1D diagnosis and a peak C-peptide level of ≥0.2 pmol/mL during a mixed meal tolerance test (MMTT). The value of 0.2 pmol/mL was chosen as it is a key and accepted threshold of C-peptide correlated with clinically important lower rates of T1D-associated short- and long-term complications (Lachin 2014, Palmer 2001, Palmer 2009).

The total study duration for each participant is up to 84 weeks. This includes a screening period of up to 6 weeks and a post-randomization period of 78 weeks. The post-randomization period includes two 12-day treatment courses separated by 6 months and a post-treatment observation period of approximately 52 weeks. The final visit takes place at Week 78.

The overall study length and timepoints for key assessments were chosen due to the natural course of remaining β cell loss following the diagnosis of T1D and study goals to demonstrate durability of effect and to confirm post-treatment safety profiles of teplizumab. At the time of diagnosis there can be substantial β cell reserves, often estimated at 10-20% but in some cases over 40% of normal β cell mass (Matveyenko 2008, Campbell-Thompson 2016). At T1D diagnosis, the majority of this reserve appears to be functionally impaired due to metabolic or immunologic (i.e, cytokine induced) stress. With exogenous insulin treatment and correction of pH, electrolyte and fluid disturbances (ie, DKA) that are often present at diagnosis, some R cell function may return for days, weeks or many months. This observation is often referred to as the “Honeymoon period” where insulin requirements can be substantially reduced and at times independence from exogenous insulin can be achieved. These effects are transient and over time, usually within a year from diagnosis, inevitably full insulin replacement is required due to autoimmune elimination of these remaining β cells. Due to the known individual variability in the natural history of β cell loss, the effect of disease modifying therapies intended to preserve β cell function is difficult to distinguish from the Honeymoon period effects during the first 12 months of T1D diagnosis.

The 18-month time point for the primary and key secondary clinical endpoints provide key data needed for the acceptance of teplizumab as a T1D disease modifying therapy into regular medical practice and is consistent with existing guidelines for endpoints recommended by the EMA and FDA. Data from T1D natural history studies and interventional trials show that 3 cell loss in those with T1D can be quite variable, especially within the weeks to months following diagnosis. As this study is enrolling participants in close proximity to T1D diagnosis (i.e. within 6 weeks) who are younger, there may be the added complexity of the consideration of the Honeymoon phenomenon (or spontaneous, transient partial remission)—that may last up to ˜1 year in the study population (Abdul-Rasoul 2006). The 18-month timing of the primary and key secondary clinical endpoints allows for a substantial amount of the inherent, natural metabolic variability due to different trajectories of β cell loss and/or transiently enhanced β function due to the Honeymoon phenomenon to be minimized—so that the true effect on teplizumab on β cell function and clinical parameters can be differentiated from chance.

Other key assessments are done at randomization, Week 26 (6 months) and Week 52 (12 months) to better understand natural history of β cell decline and the effect of teplizumab in this specific study population.

In addition, the primary and key clinical endpoints are assessed approximately 1 year after the last dose of study drug administration. The length of effect is recognized as an important property of an intermittent disease modifying therapy for T1D. A 12-month off-therapy period whilst maintaining positive metabolic and clinical effects can, at this time, be considered a reasonable time frame to substantiate an assertion of a metabolically and clinically relevant durable benefit.

Throughout this study, participants are assessed regularly via in-person interviews and physical exams, self-reports, and laboratory examinations. Assessments occur daily during the two 12-day treatment courses and regularly during the 6-month interval between courses and the 12 months following the second treatment course. The on- and off-therapy observation times in this study are well within, if not significantly beyond, the periods traditionally used to assess for safety and side effects for immune therapies approved for other autoimmune conditions, including those for pediatric indications. In doses and regimens similar to that being used in this study, teplizumab has overall been well tolerated with minimal side effects and no signals of significant short- or long-term adverse effects. It is anticipated that with additional, confirmatory data from this study, the side-effect profile of teplizumab will continue to be considered acceptable for its integration into care plans for children and adolescents newly diagnosed with T1D.

In some embodiments, T1D diagnosis is according to ADA criteria. In some embodiments, the patient diagnosed with T1D has a positive result on testing for at least one of the following T1D-related autoantibodies: Glutamic acid decarboxylase 65 (GAD65) autoantibodies, Islet antigen 2 (IA-2) autoantibodies, Zinc transporter 8 (ZnT8) autoantibodies Islet cell cytoplasmic autoantibodies (ICA) or Insulin autoantibodies (if testing obtained within the first 14 days of insulin treatment).

At the beginning of each 12-day course of study drug administration (Day 1 and Day 182), the participant's current BSA is calculated using the Mosteller formula, BSA=square root [height (cm)×weight (kg)/3600], using the height and weight of the obtained on that day.

Teplizumab and placebo are prepared according to the Pharmacy Manual provided to the site.

Polyvinyl chloride (PVC) infusion bags and tubing and normal saline should be used for study agent preparation and administration.

Two (2) mL of study drug should be drawn from the study drug vial and slowly reconstituted in 18 mL of 0.9% sodium chloride solution for injection by gentle mixing. The resulting 20 mL of 1:10 dilution is used as the initial study drug solution, which contains either placebo or teplizumab at a concentration of 100 μg/mL. This initial drug solution should then be added to a PVC infusion bag containing 25 mL 0.9% sodium chloride solution. Finally, this resulting preparation should be gently mixed before administration to the participant.

This study requires two courses of intravenous infusions and blood draws over 12 days. It is recognized that intravenous access (for infusions and blood draws for laboratory sampling) in the pediatric population that is the focus of this study may pose a challenge. Children have smaller veins than adults, veins that may be more challenging to insert catheters and they may have a significant resistance to catheter placement and/or phlebotomy.

In recognition of the above, in addition to the use of “traditional” intravenous peripheral catheters, this study permits the use of temporary, intermediate term approaches for vascular access. Specifically, “midlines” or peripherally inserted central catheter (PICC) lines may be used for study drug infusions and blood draws (if appropriate according to the properties of the access line and local, regional or national guidance).

All enrolled participants, with assistance of their health-care providers, should receive intensive diabetes management of their T1D using approved therapies according to the recommendations of American Diabetes Association (ADA) or local, regional, or national recommendations to achieve glucose levels that appear to decrease some of the short-term and long-term sequelae of TiD. Currently the glycemic targets by the ADA are focused at management strategies to achieve a HbA1c level of <7.5% (58 mmol/mol) for individuals 17 years old and younger, and <7.0% (53 mmol/mol) 18 years and older while minimizing severe or frequent hypoglycemic events.

The glycemic goal should be attempted through proper glycemic monitoring, administration of exogenous insulin, and monitoring of activity level and diet. Exogenous insulin may include rapid, intermediate, and/or long-acting insulins, administered intermittently or via the use of a personal insulin pump. Blood glucose levels should be measured at least 4 times a day, including before meals and before bedtime.

Insulin use, including the type of products, dosages, and dosing schedules, is expected to change during the course of the study. As part of routine T1D clinical care, if the caring physician judges it to be clinically appropriate, a participant's insulin dose may be increased, reduced, or even discontinued.

If participants are not meeting the glycemic goals, the study team should contact the participant's primary clinical care team about possible adjustments in the insulin regimen, referral to a registered dietitian, or other approaches that may improve the glucose control.

### Insulin Discontinuation

If a participant has achieved a HbA1c level of ≤6.5% with insulin use of ≤0.25 U/kg/day, insulin therapy can be discontinued. The participant's blood glucose and HbA1c levels should continue to be monitored per protocol, and urine ketones should be monitored once a day. During routine blood glucose monitoring, if the participant's blood glucose level exceeds 200 mg/dL (11.1 mmol/L) and/or urine ketone is moderate or greater, the participant should consult with their primary physician and/or the clinical site staff for further evaluation. If the fasting blood glucose exceeds 126 mg/dL (7 mmol/L) or HbA1c exceeds 6.5%, as documented by repeat testing, the resumption of insulin therapy should be considered.

Dosing of the study drug (teplizumab or placebo) is based on the BSA using the height and weight obtained at this visit and the Mosteller formula (BSA=square root [height (cm)×weight (kg)/3600].)

### Study Visit Week 1

Patient receives premedication of an NSAID (e.g., ibuprofen) (acetaminophen if NSAID is contraindicated) and an antihistamine (e.g., diphenhydramine) for at least the first 5 days of the treatment course, unless contraindicated by drug allergy or sensitivity. After at least 30 minutes following the premedication administration, the infusion of study drug can begin. Because there is no preservative and drug loss may occur overtime, administration of study drug should begin as soon as possible after preparation and no later than 2 hours after preparation. Study drug should be planned to be administered intravenously over 30 minutes according to standard practices, but it may be slowed if there are signs or symptoms of intolerance. When the contents of the infusion bag have been completely administered, an additional volume of saline equal to the volume contained in the infusion tubing, at the same constant rate is to be infused to ensure that all study drug has been cleared from the infusion tubing. The starting and ending times for the infusion are to be recorded.

### Day 2-12: Continued Treatment Course 1 Infusions

If there are no clinical or laboratory concerns, the patient can proceed with the next infusion as described above at least 30 minutes following administration of prophylactic NSAID (acetaminophen if NSAID is contraindicated) and antihistamine. Close monitoring is to occur during the infusions and for 60 minutes following the infusions for any signs or symptoms of intolerability or infusion reactions.

### Day 2-11

On Days 2-11, the patient is then able to leave the facility and return the following day for the next study drug infusion.

### Days 12

The Day 12 following the completion of the final infusion for this course and at least a 30-minute observation a continuous Glucose Monitoring (CGM) sensor is applied and the participant is to be given instructions on CGM monitoring care and use.

### Study Visits Weeks 4, 8, 12, and 20

The visit window for these study visits are 4 days from the target visit day. During these visits, participants return to the site for their scheduled visit and have clinical and/or laboratory assessments conducted. Of note at Week 12, a CGM sensor is applied and the participant is to be given instructions on CGM monitoring care and use.

At the Week 20 visit, give participant instructions for Week 26 4 h MMTT including overnight fast and pre-MMTT insulin dosing.

### Study Visit Week 26: 4 h MMTT and Treatment Course 2

The visit window for these study visits are ±3 days from the target visit day.

### Days 182-193

Day 182 clinical and laboratory assessments (including a 4 h MMTT) and for initiation of the second course of study drug administration.

Of specific note, height and weight are to be obtained at this visit and used for the BSA based dosing calculation for course 2. Following the guidance as with study drug course 1, the patient is to be premedicated with an oral NSAID (acetaminophen if NSAID is contraindicated) and antihistamine at least 30 minutes before the first 5 study drug infusions is started (and on an as needed basis with subsequent infusions), administration of study drug should begin as soon as possible after preparation and no later than 2 hours after preparation, and an additional volume of saline equal to the volume contained in the infusion tubing is to be infused. During the infusions and for an additional 60 minutes following the infusions participants are to be monitored for signs or symptoms of infusion reactions.

On certain days, two blood draws are obtained for teplizumab serum levels. One within 30 minutes before study agent infusion and the other within 30 minutes following study agent infusion and flush.

### Days 183-192 (Day 2-11 of Course 2 Dosing)

On Days 183-192, the participant may leave the facility and return the following day for the next study drug infusion.

### Day 193 (Day 12 of Course 2 Dosing)

Following the completion of the final infusion for this course and at least a 30-minute observation, a CGM sensor is applied and the patient is to be given instructions on CGM monitoring care and use.

### Study Visits Week 30, 34, 39, 52 and 65

The visit window for these study visits are ±4 days from the target visit day. At the Week 52 visit, a 4 h MMTT is conducted.

At the end of the Week 39, 52, and 65 visits, a CGM sensor is to be applied and additional training and instruction updates on CGM care and use is given as needed.

### Study Visits Week 39 and Week 65

Give patient instructions for Week 52 and Week 78, respectively, 4 h MMTT including overnight fast and pre-MMTT insulin dosing. At the Week 65 visit, dispense to patient CGM equipment for home application to start around Week 76.

### Study Visit Week 78

The visit window for this study visit is ±7 days from the target visit day. During this visit the 4 h MMTT is conducted.

### Mixed Meal Tolerance Tests

A 2 h MMTT is performed at screening to determine study eligibility (based on peak C-peptide level). A 4 h MMTTs is performed at randomization and at Weeks 26, 52, and 78 to obtain 4 h C-peptide AUCs and other data. A 4 h MMTT is used at and post-randomization as it has been shown to be more precise and reliable in assessing the MMTT-induced C-peptide AUC than the 2 h MMTT (Boyle 2015, Rigby 2013, Rigby 2016). Alternatively, the 2 h-MMTT is used at screening as it is sufficient to capture the peak C-peptide level needed for study entry. Samples from these assessments are assessed for C-peptide, serum glucose, and insulin. Samples are stored for potential future evaluations including but not limited to proinsulin levels. The measurements of C-peptide and glucose in serum samples are done. MMTTs are to take place in the morning between approximately 7:00 a.m. and 10:00 a.m. after an overnight fast with strict guidance on insulin use. The 2-hour MMTT takes approximately 130 minutes to perform, and the 4-hour MMTT takes approximately 250 minutes.

### Hemoglobin A1c

HbA1c is assessed as a blood test at select study visits

### Insulin Use

Patient's daily insulin use is documented by the participant in an eDiary at select times for 7 days prior to randomization and at about Weeks 12, 26, 39, 52, 65 and 78 visits. The patient records all short-, intermediate- and long-acting insulin administered as intermittent injections or use with an “insulin pump” during this time. Insulin use data are not recorded on the day before or the day of the study visit. If a patient forgets to record insulin use on one or more days before a visit, they should continue to record insulin use for up to 72 hours post-dose to obtain up to 7 days of data. Every effort should be made to collect a total of 7 days of insulin use data for all the aforementioned visits except Week 78 (final visit), as patients return the eDiary at the final visit.

### Episodes of Hypoglycemia

Clinically important and other non-severe and non-serious hypoglycemic episodes are recorded throughout the study by participants and through evaluation of glucometer readings.

### Glucose Monitoring

### (1) Intermittent Glucose Monitoring (Fingerstick)

Blood glucose levels outside of MMTT and CGM are recorded and analyzed as an endpoint at various times. As part of routine care, BG levels are usually measured by a fingerstick glucometer at least 4 times a day, including before each meal and at bedtime. At screening, participants are offered a study-supplied glucometer and glucometer strips, but participants are permitted to use their own glucometers if they choose, in which case glucose monitoring strips are not supplied. Each participant is instructed to bring their glucometer (or glucometers if they use more than one, e.g., at home and in school) to each visit for review. In addition, approximately 7 times throughout the study, participants record their BG levels before breakfast, lunch, and dinner and at bedtime for 7 consecutive days in their study eDiary prior to the randomization visit and the Weeks 12, 26, 39, 52, 65, and 78 visits. Like the recording of the insulin use data, BG data on the day before and the day of the study visit are not recorded. If a participant forgets to record fingerstick glucose measurements before a visit, they should do so for 72 hours immediately after the visit. Every effort should be made to collect a total of 7 days of BG data for all the aforementioned visits except Week 78 (final visit), as participants return the eDiary at the final visit.

### (2) Continuous Glucose Monitoring

“Continuous” glucose monitors record interstitial glucose levels (which closely approximate blood glucose values) at regular intervals, eg every 5-15 minutes depending on device. Increasingly clinical studies are supporting that such measurements and their assessments provides valuable and unique insights to glycemic control in diabetes. In this study, CGM assessments are conducted to provide key secondary clinical and exploratory endpoint data to address if and how teplizumab affects glycemic control, such as glucose excursions, time in select glucose ranges, and average daily glucose values (Steck 2014, Helminen 2016, Danne 2017). A recent international consensus statement on CGM monitoring supported the use of percentages of time in ranges (target, hypoglycemia, and hyperglycemia) and measurement of glycemic variability as key diabetes control metrics in clinical trials (Danne 2017).

CGM are used to assess glycemic control approximately 7 times throughout the study: after the completion of treatment courses at randomization and Week 26; after the visit at Weeks 12, 39, 52, and 65; and before the visit at Week 78. CGM sensors are placed by qualified study staff, and education and training on CGM use and care are given. Sensors remain in place for up to 2 weeks. If during that 2-week period a sensor comes off, it can be replaced by the participant, a knowledgeable family member/guardian, or a qualified medical professional.

To reduce any confounding factors of glucose measurements during study drug infusions, CGM sensors are placed on participants after the study drug administration has completed for Course 1 and Course 2 and other clinical and laboratory assessments have been made on the days specified in the Schedule of Events tables. At the Weeks 12, 39, 52, and 65 visits, the sensor is placed on participants after all clinical and laboratory assessments and the MMTT have completed.

Study CGM readings are not intended for medical management of participant's diabetes but can be under the supervision of a participant's health care team. Of note, routine use of the personal CGM under guidance of a participant's regular healthcare provider is permitted.

Spot-check and CGM blood glucose assessments are anticipated to include but are not be limited to mean BG, glycemic variability (BG standard deviation [SD]), maximum and minimum BG values over time and incidence and/or percent time with BG >70 but ≤180 mg/dL (>3.9 but ≤10.0 mmol/L, Level 1 (>180 but ≤250 mg/dL (>10 but ≤13.9 mmol/L)) and Level 2 HYPERglycemia (>250 mg/dL (>13.9 mmol/L)) and Level 1 (≤70 but ≥54 mg/dL (≤3.9 but ≥3.0 mmol/L)) and Level 2 (<54 mg/dL (<3.0 mmol/L)) HYPOglycemia (Seaquist 2013, International Hypoglycaemia Study Group [IHSG]2017, Agiostratidou 2017).

### Example 3. Meta-Analysis of C-peptide in Five Stage 3 T1D Studies

### Summary

Confirmatory evidence in the form of a meta-analysis was conducted using pooled C-peptide data from 5 supportive studies, all of which were randomized clinical studies: Protégé, Encore, Study 1, AbATE, and Delay. These 5 studies compared teplizumab to either placebo or standard of care in newly diagnosed patients with Stage 3 clinical T1D and had similar designs that allowed for cross-study comparisons (Table 5).

The meta-analysis evaluated the change from baseline in C-peptide AUC in a 4-hour mixed meal tolerance test (MMTT). Analysis of covariance (ANCOVA) was used to predict mean C-peptide values (least square means) as well as respective treatment differences. The meta-analysis had 2 components: one analysis was performed on all 5 studies through 1 year of follow-up, and a second analysis was performed on the 3 studies that had 2 years of follow-up.

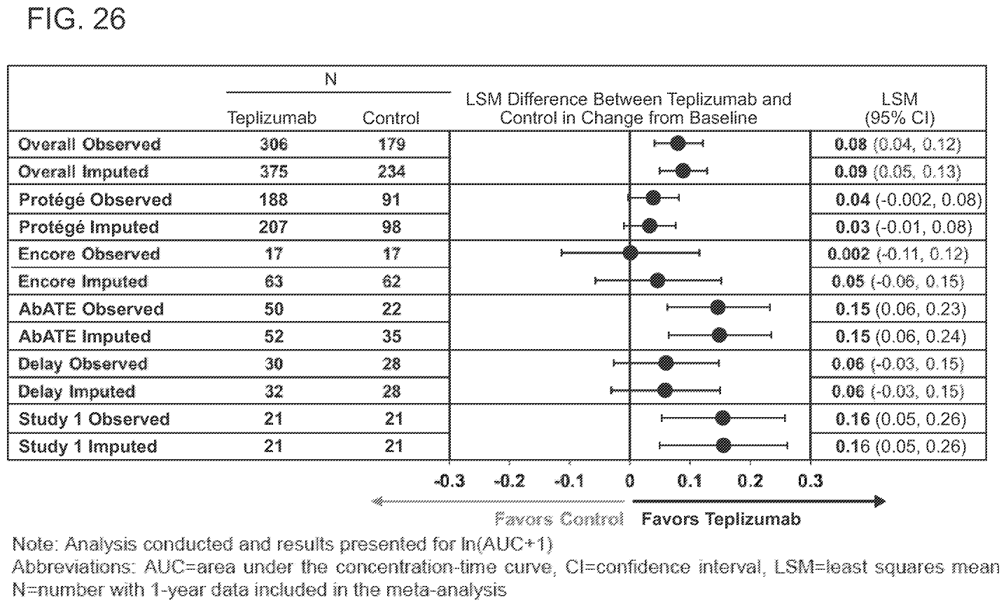

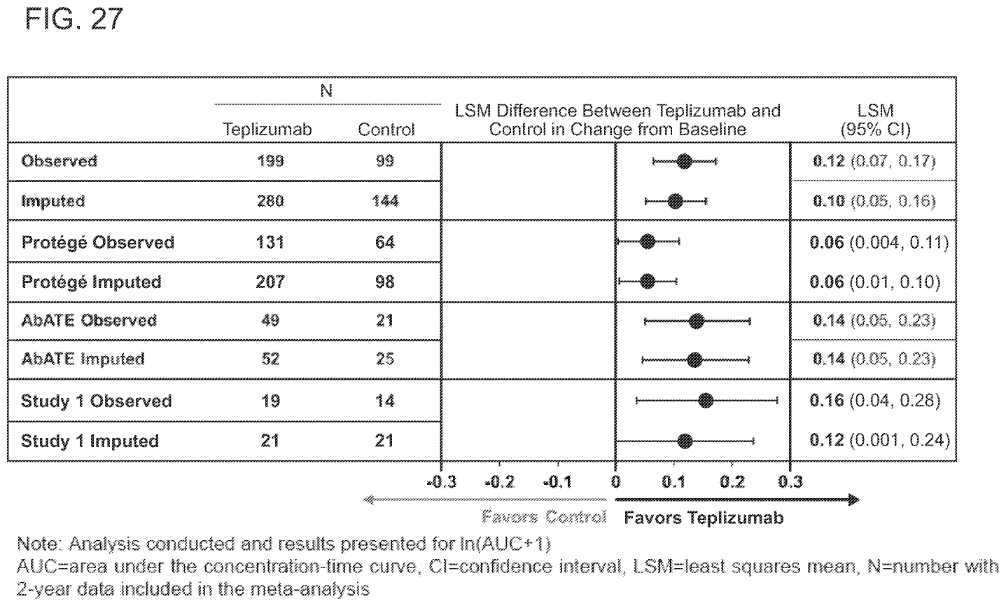

In the meta-analysis of the 1-year (FIG. 26) and 2-year C-peptide data (FIG. 27), patients treated with teplizumab showed significantly higher C-peptide levels compared to control (p<0.001 for both). This effect was consistent for observed and imputed data at both 1 year and 2 years, as well as in sensitivity analyses that assigned control data to missing data in the teplizumab group.

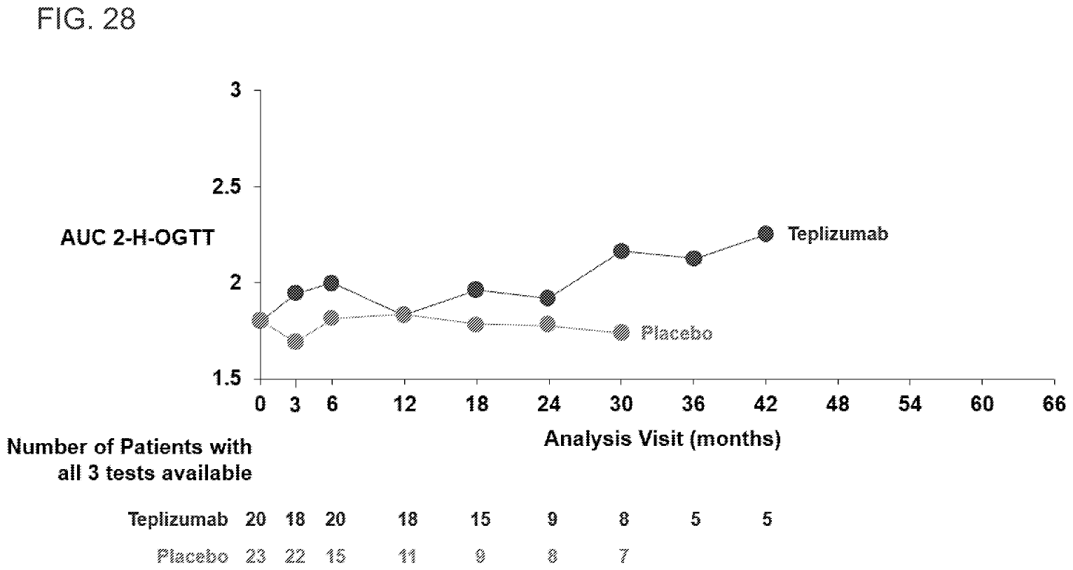

In order to assess whether C-peptide differed between those who were free of T1D and those who developed T1D, separate plots of mean C-peptide over time were developed. As can be seen in FIG. 28, those treated with teplizumab who remain free of T1D or ultimately develop T1D during the study had higher mean C-peptide values compared with their respective controls.

### Study Design

Confirmatory evidence in the form of a meta-analysis was conducted using pooled C-peptide data from 5 supportive randomized clinical studies: Protégé, Encore, Study 1, AbATE, and Delay. C-peptide AUC levels were obtained from a 4-hour MMTT.

Table 5 shows study design features across these 5 studies in Stage 3 T1D patients. These studies were chosen because they represented all the randomized studies conducted with teplizumab in Stage 3 T1D and used either placebo or standard of care as controls. A similar 14-day escalating dose regimen was used across the studies. In Study 1, a 14-day dosing regimen based on weight was subsequently modified to a 12-day dosing regimen based on BSA. However, due to apparently more AEs occurring during the early dosing period in the 12-day regimen with a 2-day ramp-up period, a 14-day regimen with a 4-day ramp-up period was adopted in subsequent clinical studies. Patients received two 14-day treatment courses in Protégé, Encore, and AbATE, and a single course of treatment at baseline in Delay and Study 1. The Protégé and Encore studies enrolled newly diagnosed patients with Stage 3 T1D in 4 treatment arms: placebo and 3 teplizumab dosing regimens (full-dose 14 days [9.0 mg/m² cumulative dose], one-third dose 14 days [˜3.0 mg/m² cumulative dose] and truncated 6-days [˜2.5 mg/m² cumulative dose]). In the meta-analysis, C-peptide data from the full-dose 14-day regimen was used. Study 1, AbATE, and Delay studies all used the full-dose 14-day regimen (9.0 mg/m² cumulative dose).

TABLE 5
Study Design Features Across Supportive Studies

| | Protégé | Encore | Study 1 | AbATE | Delay |
|---|---|---|---|---|---|
| Follow-up | Baseline, | Baseline, | Baseline, | Baseline, | Baseline, |
| | Day 140, | Day 140, | Months 6, | Months 6, | Months 6, |
| | Months 12, | Months 12, | 12, 18, 24 | 12, 18, 24 | 12 |
| | 18, 24 | 18, 24 | | | |
| C-peptide | Secondary, | Secondary, | Primary, at | Primary, at | Primary, at |
| endpoint | time not | time not | 2 years | 2 years | 1 year |
| status | specified | specified | | | |
| Dosing | 14 days | 14 days | 12 or 14 days | 14 days | 14 days |
| schedule | | | | | |
| Regimen | 2 courses: | 2 courses: | 1 course: | 2 courses: | 1 course: |
| | Baseline, | Baseline, | Baseline | Baseline, | Baseline |
| | 6 months | 6 months | | 12 months | |
| Design | Randomized, | Randomized, | Randomized, | Randomized, | Randomized, |
| | double- | double-blind | open-label | open-label | double-blind |
| | blind | | | | |
| Control | Placebo | Placebo | Standard care | Standard care | Placebo |
| group | | | | | |
| Number of | Placebo: 98 | Placebo: 62 | Control: 21 | Control: 25 | Placebo: 27 |
| patients* | Teplizumab: 453 | Teplizumab: 192 | Teplizumab: 21 | Teplizumab: 52 | Teplizumab: 31 |

The patients enrolled in these studies (Table 7) were representative of the newly diagnosed TTD patient population, excluding those with significant medical history, clinical abnormalities, or active infection. Key inclusion criteria were similar across the studies. C-peptide level at study entry was 0.2 nmol/L in AbATE, Delay, and Study 1 and detectable levels for Protege and Encore.

TABLE 7
Key Inclusion Criteria Across the Supportive Studies

| | Protégé | | Encore | | Study 1 | | AbATE | | Delay | |
|---|---|---|---|---|---|---|---|---|---|---|
| Age at entry | 8-35 | years | 8-35 | years | 7.5-30 | years | 8-30 | years | 8-30 | years |
| Time of T1D | ≤12 | weeks | ≤12 | weeks | ≤6 | weeks | ≤8 | weeks | 4-12 | months |

| diagnosis to | | | | | | | | |
|---|---|---|---|---|---|---|---|---|
| treatment | | | | | | | | |
| Autoantibodies1 | anti-ICA512, | anti-ICA512, | anti-GAD65, | | ICA, anti- | | ICA, anti- | |
| | IA-2, anti- | IA-2, anti- | anti-ICA512, | | GAD65, anti- | | GAD65, anti- | |
| | GAD65, IAA | GAD65, IAA | IAA | | ICA512 | | ICA512 | |
| C-peptide level | detectable | detectable | ≥0.2 | nmol/L | ≥0.2 | nmol/L | ≥0.2 | nmol/L |

| | | | | | | | | | |
|---|---|---|---|---|---|---|---|---|---|
| Weight | ≥36 | kg | ≥36 | kg | N/A | ≥25 | kg | ≥27.5 | kg |

The primary endpoint of the meta-analyses was the change from baseline in C-peptide AUC in a 4-hour MMTT. Each study used the same sample collection time points during the MMTTs to calculate C-peptide AUC.

Meta-Analysis of Change from Baseline in C-Peptide AUC in a 4-Hour MMTT

Patients in the teplizumab group had greater preservation of C-peptide (ie, smaller decreases from baseline) at 1 year and 2 years of follow-up. This effect was consistent for observed data and imputed data (p<0.0001 for both analyses). Furthermore, the conservative sensitivity analysis applying control-based imputation (assigning control data to missing teplizumab data) was also significant (p<0.0001).

The results for the 1-year and 2-year meta-analyses are shown in forest plots in FIG. 26 and FIG. 27, respectively. Both forest plots show that the observed (existing) and imputed analyses yielded consistent effects of teplizumab in preserving C-peptide AUC levels. In the 1-year forest plot, teplizumab treatment was consistently more effective than placebo in all studies, except Encore. The result in the Encore study was expected, as the study was modified before its completion due the companion Phase 3 study, Protégé, not meeting its 1-year primary endpoint, resulting in a large amount of missing data requiring the largest amount of imputation. Approximately 75% (93/125) of the MMTTs were missing. The primary endpoint of the Protégé study was a novel unvalidated composite endpoint focused on metabolic parameters (HbA1c and insulin use).

In the forest plot of 2-year data (FIG. 27), teplizumab treatment significantly preserved C-peptide AUC levels compared with placebo in all 3 studies with 2-year data.

### Example 4. Insulin Use in Five Stage 3 T1D Studies

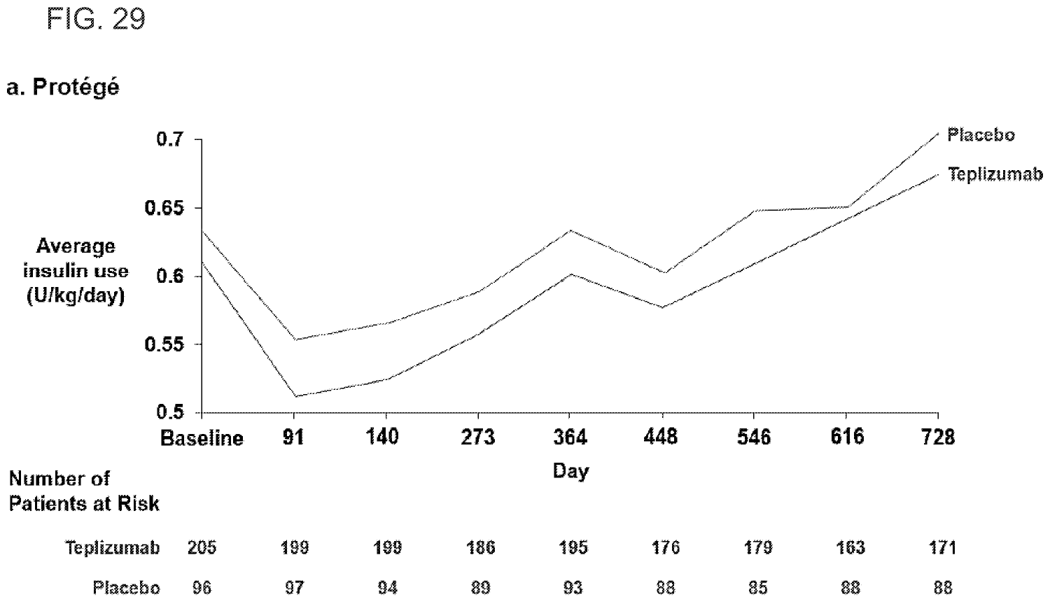

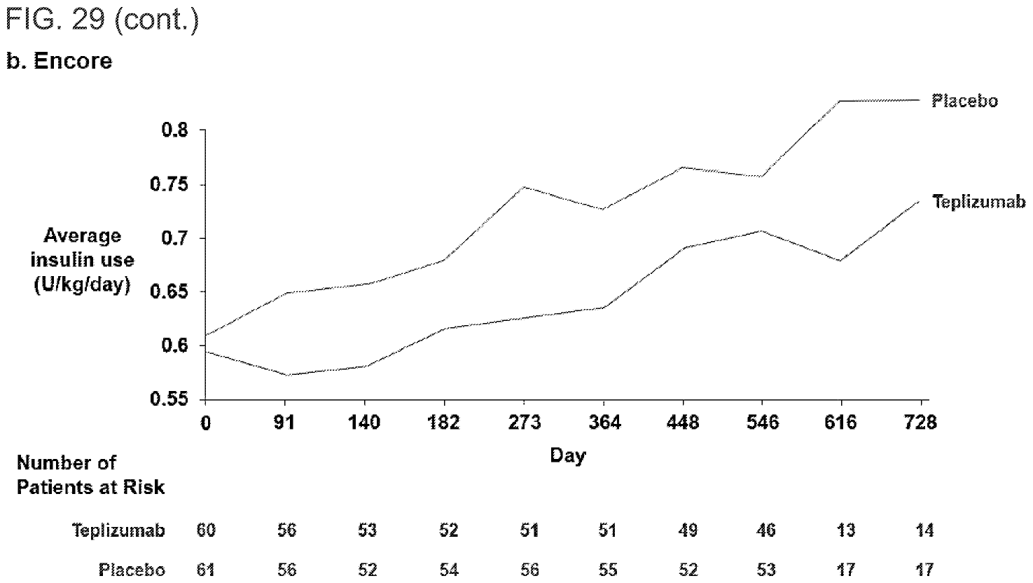

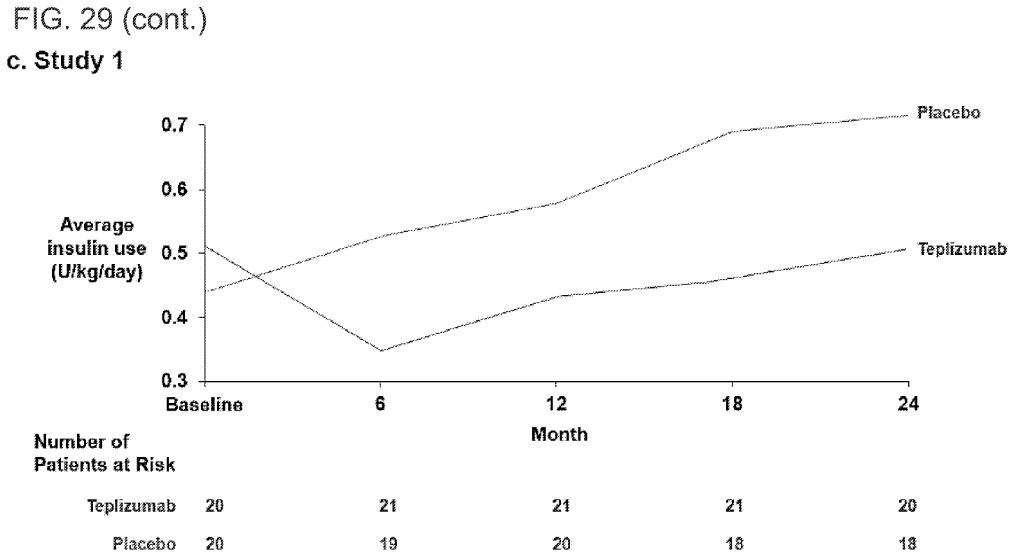

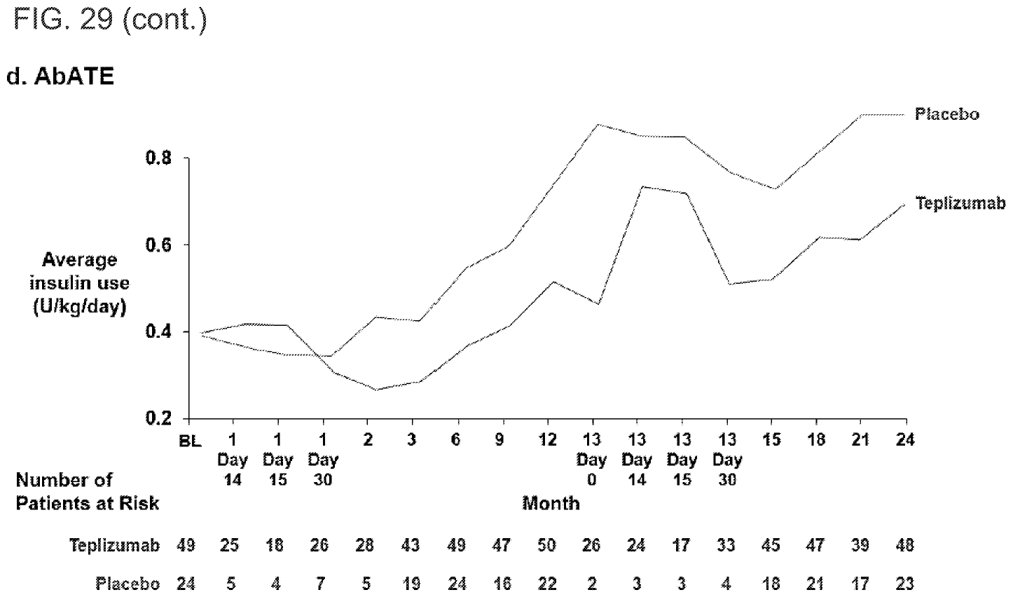

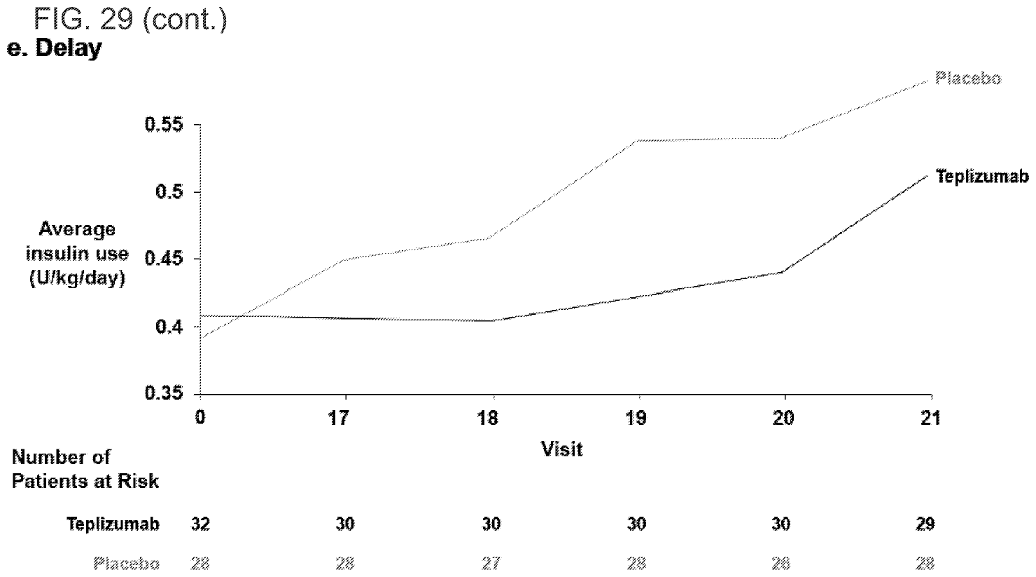

In the same 5 studies included in the C-peptide meta-analysis in Example 3, exogenous insulin use was evaluated individually in each study. The mean insulin use over each timepoint in each study was numerically lower in teplizumab-treated patients compared to placebo (FIG. 29). In 2 of the studies (AbATE, Study 1) the difference was statistically significant.

Specifically, in all 5 studies, the mean insulin use over each timepoint was lower in teplizumab patients compared to placebo patients (FIG. 29). Three studies (AbATE, Delay, and Study 1) showed that teplizumab treatment consistently led to statistically significantly lower levels of insulin requirement compared with placebo (Herold et al 2013a; Herold et al 2005; Herold et al 2013b). The insulin use in the teplizumab group was also lower compared to the placebo group but did not achieve statistical significance in the Protégé and Encore studies. Thus, teplizumab treatment preserves C-peptide levels as reflected by greater endogenous insulin production and less exogenous insulin requirement.

Overall, these data support that teplizumab preserves beta cell function, as measured by C-peptide levels, and correspondingly, endogenous insulin production, resulting in a lower need for exogenous insulin.

### Example 5: Clinical Pharmacokinetics and Pharmacodynamics

Mechanism of Action: Teplizumab is a humanized monoclonal antibody that targets the cluster of differentiation 3 (CD3) antigen, which is co-expressed with the T-cell receptor (TCR) on the surface of T lymphocytes. Though the mechanism of action of teplizumab for the proposed indication has not been confirmed, it appears to involve weak agonistic activity on signaling via the TCR-CD3 complex, which is thought to expand regulatory T-cells and re-establish immune tolerance.

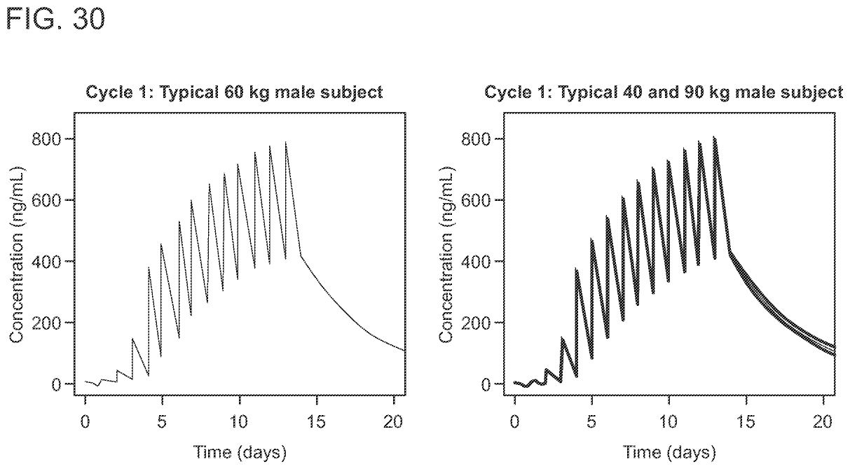

Pharmacokinetics: FIG. 30 shows plots of predicted mean teplizumab concentrations over time using a 14-day intravenous (IV) dosing regimen with a 4-day ramp-up followed by repeated doses of 826 μg/m² on Days 5 to 14. The left panel represents a typical 60 kg male subject and the right panel represents a typical 40 kg and 90 kg male subject. Body surface area (BSA)-based dosing normalizes the exposure across body size.

The repeated IV infusions resulted in increasing serum teplizumab levels, although steady-state PK was not achieved at the end of dosing (Day 14 with this dosing regimen). The average accumulation ratio for area under the curve (AUC) between Day 5 and Day 14 was 3.4. The predicted mean (+SD) total AUC for the 14-day dosing regimen was 6421±1940 ng·day/mL with Cmax and Cmin of 826±391 and 418±225 ng/mL, respectively, on Day 14.

Distribution: The central and peripheral volume of distribution from population PK analysis was 3.4 L and 6.9 L, respectively.

Elimination: Teplizumab clearance is not dose-proportional, likely driven by its saturable binding to CD3 receptors on the T-cell surface. Teplizumab is expected to be degraded into smaller peptide fragments by catabolic pathways. The clearance of teplizumab following the 14-day dosing regimen was estimated from population PK analysis to be 2.3 L/day, with a terminal half-life of approximately 4 days.

The planned commercial drug product is manufactured in a different facility from the clinical trial product and was not used in the clinical studies submitted to support efficacy and safety. A single-dose PK bridging study was conducted in healthy volunteers that evaluated the biocomparability of the commercial drug product with the clinical trial drug product. The mean AUC0-inf for the commercial product was less than half (48.5%, 90% CI: 43.6 to 54.1) of the AUC0-inf for the product used in the primary efficacy study. The reason for this difference seems to be faster clearance of the drug from circulation rather than differences in the strength of the product, as similar concentrations were observed immediately following IV infusion (Cmax of the commercial product was 94.5% (90% CI: 84.5 to 106) of that observed in the clinical trial drug product).

### Example 6. Adverse Events

Adverse events associated with teplizumab administration are also being studied. Notably, while teplizumab does not have an overall infection safety signal to date, patients receiving the 12-day dosing regimen (1 or 2 courses) instead of 14-day regimen appear to report fewer numeric adverse events of infection, based on the data from completed studies (Table 6).

TABLE 6
TEAEs by number of doses, SOC and Preferred t [symbol] [symbol] Safety population [symbol]
-- Event count

| | | All doses | 12 doses | Teplizu [symbol] |
|---|---|---|---|---|
| | | received | received | Full Regimen |
| | | [symbol] N = 6370 [symbol] | [symbol] N = 52 [symbol] | [symbol] N = [symbol] 345 [symbol] |
| System organ class | Preferred term | n [symbol] % [symbol] | n [symbol] % [symbol] | n [symbol] % [symbol] |
| Skin and subcutaneous tissue disorders | Vitiligo | 0 | 0 | 1 (0.0) |
| Infe [symbol] ions and infestations | -Total | 526 (0.3) | 2 (3.0) | 600 (7.9) |
| | Upper respiratory tract infection | 149 (2.3) | 0 | 184 (2.2) |
| | Na [symbol] ph [symbol] ry [symbol] tis | 44 (0.7) | 0 | 6 [symbol] (0. [symbol] ) |
| | [symbol] yngitis | 36 (0.6) | 0 | 39 (0.5) |
| | Sinusitis | 22 (0.3) | 0 | 2 [symbol] (0.3) |
| | Rhinitis | 17 (0.3) | 0 | 20 (0.2) |
| | Gast [symbol] tis viral | 13 (0.2) | 0 | 10 (0.2) |
| | [symbol] yngitis streptococcal | 12 (0.2) | 0 | 1 [symbol] (0.2) |
| | Epstein-Barr vir [symbol] | 10 (0.2) | 0 | 1 [symbol] (0.2) |
| | Influenza | 14 (0.2) | 0 | 14 (0.2) |
| | Conjunctivitis | 11 (0.2) | 0 | 13 (0.2) |
| | Ear infection | 0 (0.1) | 0 | 13 (0.2) |
| | Epstein-Barr virus [symbol] nfection | 11 (0.2) | 1 (1.9) | 13 (0.2) |
| | Viral upper respiratory tract infection | 11 (0.2) | 0 | 12 (0.1) |
| | Gastro [symbol] tis | [symbol] (0.1) | 0 | 10 (0.1) |

Note:
The denominator in percent calculation is the number of subjects in each treatment group and subgroup level for subject count and the number of events in each treatment group and subgroup level for event count.
Note:
Delay subjects initially randomized to the Placebo arm, who were later also eligible for the open-label cycle 2 administration of [symbol] , arm included only for the [symbol] period.
Note:
Subjects were considered under ‘All doses [symbol] ’ if they had 14 doses administered during cycle 1, or they had 14 doses administered during both cycle 1 and 2 if they participated in two cycles. If subjects had 12 doses administered during cycle 1 or the sum of doses during cycles 1 and 2 was 12, they were considered under [symbol] 12 doses received [symbol] .
Note: [symbol]
organ classes [symbol] and preferred terms are based on Med [symbol] version 23.0.
Note:
TEAE—Treatment-Emergent Adverse Event. [symbol]
indicates data missing or illegible when filed

An established (>1 year post-diagnosis) Stage-4 T1D patient with residual beta-cell mass, as evidenced by stimulated C-peptide levels lower than about 1 ng/ml in blood yet detectable, receives a 12-day course of teplizumab IV to reduce immunological auto-reactivity against beta cells while starting a daily oral treatment with a DYRK1A inhibitor at an effective dose. The DYRK1A inhibitor acts on the small but existing residual beta-cells inducing proliferation. As a result of the teplizumab treatment at the start of the therapy, the newly formed beta cells are not attacked by the immune system and the patient's needs for exogenous insulin is either reduced by at least 20% or fully eliminated.

In a variation of this example, the patient receives a second or more treatment courses (e.g. 12-day course) of teplizumab separated by a period ranging from about 6 months to about 1 year to prevent the immune system from attacking newly formed beta cells. The new course of teplizumab occurs while the patient continues expanding their beta cell mass by taking orally a daily effective dose of DYRK1A inhibitors. Alternatively, the new course of teplizumab occurs after the patient has stopped the DYRK1A inhibitors treatment and is administered to ensure that the immune system does not react against the expanded beta-cell mass.

### Example 8: Treatment of Newly Diagnosed, Symptomatic, Insulin-Dependent Stage 3 T1D Patient

A newly diagnosed, symptomatic, insulin-dependent stage 3 T1D patient with significant residual beta-cell mass as evidenced by C-peptide levels higher than about 0.2 ng/ml in blood receives a 12-day course with daily subcutaneous administrations of a pharmaceutical composition comprising a combination of anti-CD3 antibody (e.g. teplizumab) and DYRK1A inhibitors such that the active agents are administered at a therapeutically effective amount to the subject in need thereof in each dose. After that the 12-day course, the patient continues receiving a weekly dose of a therapeutically effective subcutaneous amount of DYRK1A inhibitors or a therapeutically effective oral amount of DYRK1A inhibitors until the patient's need for exogenous insulin is reduced by at least 20% or fully eliminated.

In a variation of this example, the patient receives from weekly to monthly subcutaneous doses of DYRK1A after the first 12-day course of teplizumab. In another variation, the patient receives additional 12-day course of the combined anti-CD3 and DYRK1A inhibitors every 6 months while staying on the from weekly to monthly treatment of DYRK1A inhibitors monotherapy.

### Example 9: Treatment of Pre-Symptomatic Stage-2 T1D Patient

A pre-symptomatic stage-2 T1D patient with reduced functional beta-cell mass as evidenced by the presence of dysglycemia upon glycemic challenge receives a 12-day course of teplizumab IV to reduce immunological auto-reactivity against beta cells and prevent further progression of the disease. One month later, the patient starts DYRK1A inhibitors therapy orally to expand the pre-existing beta cell mass until dysglycemia is reduced by about 50% compared to baseline or eliminated.

In a variation of this example, an additional 12-day course of teplizumab is administered after from about 6 months to about 1 year after the first 12-day teplizumab course to avoid rejection of the newly formed beta cells by the patient's immune system.

In a variation of this example, the effective amount of DYRK1A inhibitors is administered orally one to four times a day, orally once a week, orally once every two weeks, orally once every three weeks or orally once a month.

Alternatively, the effective amount of DYRK1A inhibitors is administered orally from once to several times a day for one week each month.

Alternatively, the effective amount of DYRK1A inhibitors is administered orally from once to several times a day for two consecutive weeks each month.

Alternatively, the effective amount of DYRK1A inhibitors is administered orally from once to several times a day on alternative weeks.

### Example 10: Treatment of Established Stage-4 T1D Patient

An established (>1 year post-diagnosis) Stage-4 T1D patient with residual beta-cell mass as evidenced by stimulated levels of C-peptide detectable yet lower than about 1 ng/ml in blood receives a 12-day course of teplizumab IV while starting a continuous administration of an effective dose of DYRK1A inhibitors using an infusion pump.

In a variation of this example, the patient receives a second or more courses of teplizumab (e.g. 12-day course) separated by from about 6 months to about 1 year to prevent the immune system from attacking newly formed beta cells. The second or more courses of teplizumab could occur while the patient continues expanding their beta cell mas while wearing the infusion pump.

In a variation of this example, the effective amount of DYRK1A inhibitors is administered by an infusion pump that is worn continuously for 7-14 days of each month.

Alternatively, the effective amount of DYRK1A inhibitors is administered by an infusion pump that is worn alternative days.

Alternatively, the effective amount of DYRK1A inhibitors is administered by an infusion pump that is worn 1-5 days each week.

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

### REFERENCES

Aarnodt K I. Aramandla R, Brown J J, Fiaschi-Taesch N, Wang P, Stewart A F, Brissova M, Powers A C. Development of a reliable automated screening system to identify small molecules and biologics that promote human β-cell regeneration. Am J Physiol Endocrinol Metab. 2016 Nov. 1; 311(5):E859-E868.
Abdolazimi Y, Zhao Z, Lee S, et al. CC-401 Promotes n-Cell Replication via Pleiotropic Consequences of DYRK1A/B Inhibition. Endocrinology. 2018; 159(9): 3143-3157.
Abdul-Rasoul M, Habib H, Al-Khouly M. “The honeymoon phase” in children with type 1 diabetes mellitus: frequency, duration, and influential factors. Pediatr Diabetes. 2006; 7(2):101-107.
Ackeifi C, Swartz E, Kumar K, Liu H, Chalada S, Karakose E, Scott D K, Garcia-Oca5a A, Sanchez R, DeVita R J, Stewart A F, Wang P. Pharmacologic and genetic approaches define human pancreatic β cell mitogenic targets of DYRK1A inhibitors. JCI Insight. 2020 Jan. 16; 5(1):e132594.
Ablamunits V, Bisikirska B, Herold K C. Acquisition of regulatory function by human CD8+ T cells treated with anti-CD3 antibody requires TNF. Eur J Immunol. 2010; 40(10):2891-2901.
Agiostratidou G, Anhalt H, Ball D, et al. Standardizing clinically meaningful outcome measures beyond HbA1c for type 1 diabetes: a consensus report of the American Association of Clinical Endocrinologists, the American Association of Diabetes Educators, the American Diabetes Association, the Endocrine Society, JDRF International, The Leona M. and Harry B. Helmsley Charitable Trust, the Pediatric Endocrine Society, and the T1D Exchange. Diabetes Care. 2017; 40(12):1622-1630.
Akirav E M, Kushner J A, Herold K C. β mass and type 1 diabetes: Going, going, gone?Diabetes. 2008; 57:2883-2888.
Ambery P, Donner T W, Biswas N, et al. Efficacy and safety of low-dose otelixizumab anti-CD3 monoclonal antibody in preserving C-peptide secretion in adolescent type 1 diabetes: DEFEND-2, a randomized, placebo-controlled, double-blind, multi-centre study. Diabetic Medicine. 2013; 31(4): 399-402.
American Diabetes Association. 12. Children and Adolescents: Standards of medical Care in Diabetes-2018. Diabetes Care. 2018; 41 (Suppl. 1):S126-S136.
Atkinson M A. Type 1 diabetes. Lancet. 2014; 383(9911):69-82 Aronson R, Gottlieb P, Christiansen J, et al. Low-Dose Otelixizumab Anti-CD3 Monoclonal Antibody DEFEND-1 Study: Results of the Randomized Phase III Study in Recent-Onset Human Type 1 Diabetes. Emerging Technologies and Therapeutics. 2014; 27(10):2746-2754.
Bettini R, Cocconi D. Handbook of Pharmaceutical Excipients. Pharmaceutical Press. 2001; 71(3):352-353.
Bisikirska B, Colgan J, Luban J, Bluestone, J A, Herold K C. TCR stimulation with modified anti-CD3 mAb expands CD8+ T cell population and induces CD8+CD25+ Tregs. J Clin Invest. 2005; 115(10):2904-2913.
Bingley, Polly J. Interactions of Age, Islet Cell Antibodies, Insulin Autoantibodies, and First-Phase Insulin Response in Predicting Risk of Progression to IDDM in ICA+ Relatives: The ICARUS Data Set. Diabetes. 1996; 45(12):1720-1728.
Bluestone J A, Herold K, and Eisenbarth G. Genetics, pathogenesis and clinical interventions in type 1 diabetes. Nature. 2010; 464(7293):1293-1300.
Bolt S, Routledge E, Lloyd I et al. The generation of a humanized, non-mitogenic CD3 monoclonal antibody which retains in vitro immunosuppressive properties. European Journal of Immunology. 1993; 23(2):403-411.
Boyle K D, Keyes-Elstein L, Ehlers M R, et al. Two- and four-hour tests differ in capture of C-peptide responses to a mixed meal in type 1 diabetes. Diabetes Care. 2016; 39:e76-78.
Bradley C, Loewenthal K, Woodcock A, et al. Development of the diabetes treatment satisfaction questionnaire (DTSQ) for teenagers and parents: the DTSQ-Teen and the DTSQ-Parent. Diabetologia. 2009; 52: (Suppl 1) S397, Abstract 1013.
Buchwald H, Rohde T D, Schneider P D, et al. Long-term, continuous intravenous heparin administration by an implantable infusion pump in ambulatory patients with recurrent venous thrombosis. Surgery. 1980; 88(4):507-16.
Campbell-Thompson M, Fu A, Kaddis J S, et al. Insulitis and β-cell mass in the natural history of type 1 diabetes. Diabetes. 2016; 65:719-731.
Cleek et al. Biodegradable Polymeric Carriers For A bFGF Antibody For Cardiovascular Application. Pro. Int'l. Symp. Control. Rel. Bioact. Mater. 1997; 24:853-854.
Daifotis A, koeing S, Chatenoud L, et al. Anti-CD3 clinical trials in type 1 diabetes mellitus.
Clinical Immunology. 2013; 149(3) Part A: 268-278.
Danne T, Nimri R, Battelino T, et al. International consensus on use of continuous glucose monitoring. Diabetes Care 2017; 40:1631-1640.
Diabetes Control and Complications Trial Research Group. Hypoglycemia in the Diabetes Control and Complications Trial. Diabetes. 1997; 46(2): 271-286.
Diabetes Control and Complications Trial Research Group. The effect of intensive treatment of diabetes on the development and progression of long-term complications in insulin-dependent diabetes mellitus. N Engl J Med. 1993; 329(14):977-86.
DiMeglio L A, Acerini C L, Codner E, et al. ISPAD Clinical Practice Consensus Guidelines 2018: Glycemic control targets and glucose monitoring for children, adolescents, and young adults with diabetes. Pediatr Diabetes. 2018 October; 19 Suppl 27:105-114.
Dirice E, Walpita, D, Vetere A et al. Inhibition of DYRK1A Stimulates Human R-Cell Proliferation. Diabetes. 2016; 65(6):1660-1671.
Driscoll K A, Raymond J, Naranjo D, et al. Fear of hypoglycemia in children and adolescents and their parents with type 1 diabetes. Curr Diab Rep. 2016; 16(8):77.
During M J, Freese A, Sabel B A, et al. Controlled release of dopamine from a polymeric brain implant: In vivo characterization. Annals of Neurology. 1989; 25(4): 351-356.
European Medicines Agency. Ethical Considerations for Clinical Trials on Medicinal Products Conducted with the Paediatric Population. 2008. https://ec.europa.eu/health/sites/health/files/files/eudralex/vol-10/ethical_considerations_en.pdf. Accessed Sep. 27, 2018.
Fonolleda M, Murillo M, Vizquez F, et al. Remission phase in paediatric type 1 diabetes: new understanding and emerging biomarkers. Horm Res Paediatr. 2017; 88(5):307-315.
Gitelman S E, Gottlieb P A, Rigby M R, et al. Antithymocyte globulin treatment for patients with recent-onset type 1 diabetes: 12-month results of a randomised, placebo-controlled, phase 2 trial. Lancet Diabetes Endocrinol. 2013; 1(4):306-16.
Gonder-Frederick L, Nyer M, Shepard J A. Assessing fear ofhypoglycemia in children with Type 1 diabetes and their parents. Diabetes Manag (Lond). 2011; 1(6): 627-639.
Greenbaum C J, Beam C A, Boulware D et al. Fall in C peptide during first 2 years from diagnosis: evidence of at least two distinct phases from composite type 1 diabetes TrialNet data. Diabetes. 2012; 61:2066-2073.
Greenbaum C J, Cuthbertson D, Krischer J P, et al. Type 1 Diabetes Manifested Solely by 2-h Oral Glucose Tolerance Test Criteria. Diabetes. 2001; 50(2):470-476.
Guglielmi C, Williams S R, Del Toro R et al. Efficacy and safety of otelixizumab use in new-onset type 1 diabetes mellitus. Drug Evaluation. 2016; 16(6): 841-846.
Hagopian W, Ferry R J Jr, Sherry N et al, Protégé Trial Investigators. Teplizumab preserves C-peptide in recent-onset type 1 diabetes: two-year results from the randomized, placebo-controlled Protégé trial. Diabetes. 2013; 62(11):3901-8. doi: 10.2337/db13-0236. Epub 2013 June 25.
Helminen O, Pokka T, Tossavainen P, et al. Continuous glucose monitoring and HbA1c in the evaluation of glucose metabolism in children at high risk for type 1 diabetes mellitus. Diabetes Res Clin Pract. 2016; 120:89-96.
Herold K C, Hagopian W, Auger J A, Poumian-Ruiz E, Taylor L, Donaldson D, Gitelman S E, Harlan D M, Xu D, Zivin R A, Bluestone J A. Anti-CD3 monoclonal antibody in new-onset type 1 diabetes mellitus. N Engl J Med. 2002 May 30; 346(22):1692-1698.
Herold K C, Gitelman S E, Umest M et al. A single course of anti-CD3 monoclonal antibody hOKT3γ1 (Ala-Ala) results in improvement in C-peptide responses and clinical parameters for at least 2 years after onset of type 1 diabetes. Diabetes. 2005; 54:1-7.
Herold K C, Gitelman S E, Ehlers M R, et al. Teplizumab (anti-CD3 mAb) treatment preserves C-peptide responses in patients with new-onset type 1 diabetes in a randomized controlled trial: metabolic and immunologic features at baseline identify a subgroup of responders. Diabetes. 2013(a); 62(11):3766-3774.
Herold K C, Gitelman S E, Willi S M, et al. Teplizumab treatment may improve C-peptide responses in participants with type 1 diabetes after the new-onset period: a randomised controlled trial. Diabetologia. 2013(b); 56(2):391-400.
Herold K C, Bundy B N, Long S A, et al. An Anti-CD3 Antibody, Teplizumab, in Relatives at Risk for Type 1 Diabetes. N Engl J Med. 2019 Jun. 9. doi: 10.1056/NEJMoa1902226. [Epub ahead of print]
Howard M A, Gross A, Grady M S et al. Intracerebral drug delivery in rats with lesion-induced memory deficits. Journal of Neurosurgery. 1989; 71(1):105-112.
Huo L, Harding J L, Peeters A, et al. Life expectancy of type 1 diabetic patients during 1997-2010: a national Australian registry-based cohort study. Diabetologia. 2016; 59(6):1177-85.
International Hypoglycaemia Study Group. Glucose Concentrations of Less Than 3.0 mmol/L (54 mg/dL) Should Be Reported in Clinical Trials: A Joint Position Statement of the American Diabetes Association and the European Association for the Study of Diabetes. Diabetes Care. 2016; dc162215.
Joint Commission. High Alert Medications and Patient Safety. Sentinel Event Alert. Issue 11. Nov. 19, 1999. https://www.jointcommission.org/assets/i/18/SEA_11.pdf. Accessed Aug. 21, 2018.
Karvonen M, Viik-Kajander M, Moltchanova E, et al. Incidence of childhood type 1 diabetes worldwide. Diabetes Mondiale (DiaMond) Project Group. Diabetes Care. 2000; 23(10):1516-1526.
Bart Keymeulen, M. D., Ph.D., Evy Vandemeulebroucke, M. D., Anette G. Ziegler, M. D., Ph.D., et al. Insulin Needs after CD3-Antibody Therapy in New-Onset Type 1 Diabetes. The New England Journal of Medicine. 2005; 352:2598-2608.
Keymeulen B, Walter M, Mathieu C, et al. Four-year metabolic outcome of a randomised controlled CD3-antibody trial in recent-onset type 1 diabetic patients depends on their age and baseline residual beta cell mass. Diabetologia. 2010; 53:614-623.
Keymeulen B, Candon S, FafipKremer S, et al. Transient Epstein-Barr virus reactivation in CD3 monoclonal antibody-treated patients. Blood. 2010; 115(6):1145-1155.
Kuhtreiber W M, Washer S L, Hsu E, et al. Low levels of C-peptide have clinical significance for established Type 1 diabetes. Diabet Med. 2015 October; 32(10):1346-53.
Kumar K, Wang P, Sanchez R, Swartz E A, Stewart A F, DeVita R J. Development of Kinase-Selective, Harmine-Based DYRK1A Inhibitors that Induce Pancreatic Human beta-Cell Proliferation. J Med Chem. 2018 Sep. 13; 61(17):7687-7699.
Kumar K, Wang P, Wilson J, Zlatanic V, Berrouet C, Khamrui S, Secor C, Swartz E A, Lazarus M, Sanchez R, Stewart A F, Garcia-Ocana A, DeVita R J Synthesis and Biological Validation of a Harmine-Based, Central Nervous System (CNS)-Avoidant, Selective, Human beta-Cell Regenerative Dual-Specificity Tyrosine Phosphorylation-Regulated Kinase A (DYRK1A) Inhibitor. J Med Chem. 2020 Mar. 26; 63(6):2986-3003.
Lachin J M, McGee P, Palmer J P; DCCT/EDIC Research Group. Impact of C-peptide preservation on metabolic and clinical outcomes in the Diabetes Control and Complications Trial. Diabetes. 2014; 63(2):739-748.
Laitinen O H, Honkanen H, Pakkanen 0, et al. Coxsackievirus B1 is associated with induction of β-cell autoimmunity that portends type 1 diabetes. Diabetes. 2014; 63(2):446-455.
Lam et al. Microencapsulation of recombinant humanized monoclonal antibody for local delivery. Proc. Int'l. Symp. Control Rel. Bioact. Mater. 1997; 24:759-760.
Langer, Robert. New Methods of Drug Delivery. Science. 1990; 249(4976):1527-1533.
Langer R, Peppas N. Chemical and Physical Structure of Polymers as Carriers for Controlled Release of Bioactive Agents: A Review. Journal of Macromolecular Science, Part C >Polymer Reviews. 1983; 23(1):61-126.
Langer R S, Wise D L. Medical Applications of Control Release. 1985; 2(1): 115-138.
Lebastchi J, Deng S, Lebastchi A H, et al. Immune therapy and β-cell death in type 1 diabetes. Diabetes. 2013; 62(5):1676-1680.
Levy R J, Wolfru, J, Schoen F J, et al. Inhibition of Calcification of Bioprosthetic Heart Valves by Local Controlled-Release Diphosphonate. Science. 1985; 228(4696): 190-192.
Lin A, Northam E A, Werther G A, Cameron F J. Risk factors for decline in I Q in youth with type 1 diabetes over the 12 years from diagnosis/illness onset. Diabetes Care. 2015; 38:236-242.
Lind M, Svensson A M, Kosiborod M, et al. Glycemic control and excess mortality in type 1 diabetes. N Engl J Med. 2014; 371:1972-1982.
Long S A, Thorpe J, DeBerg H A, et al. Partial exhaustion of CD8 T cells and clinical response to teplizumab in new-onset type 1 diabetes. Sci. Immunol. 2016; 1(5):1-23.
Long S A, Thorpe J, Herold K C, et al. Remodeling T cell compartments during anti-CD3 immunotherapy of type 1 diabetes. Cell Immunol. 2017 September; 319:3-9.
Lopez-Berestein G, Fidlet I, et al. Liposomes in the therapy of infectious diseases and cancer. Food and Agriculture Organization of the United Nations. 1989; 353-365.
Ludvigsson J, Carlsson A, Deli A, et al. Decline of C-peptide during the first year after diagnosis of Type 1 diabetes in children and adolescents. Diabetes Res Clin Pract. 2013; 100(2):203-209.
Masharani U B, Becker J. Teplizumab therapy for type 1 diabetes. Expert Opin Biol Ther. 2010; 10(3): 459-465.
Matveyneko A V and Butler P C. Relationship between β cell mass and diabetes onset. Diabetes Obes Metab. 2008; 10:23-31.
Mayfield, Jennifer. Diagnosis and Classification of Diabetes Mellitus: New Criteria. American Family Physician. 1998; 58(6): 1355-62, 1369-70.
McCulloch D K, Bingley P J, Colman P G, et al. Comparison of Bolus and Infusion Protocols for Determining Acute Insulin Response to Intravenous Glucose in Normal Humans. Diabetes Care. 1993; 16(6):911-915.
Mittermayer F, Caveney E, De Oliveira C, et al. Addressing Unmet Medical Needs in Type 1 Diabetes: A Review of Drugs Under Development. Curr Diabetes Rev. 2017; 13(3):300-314.
Monaghan M, Helgeson V, Wiebe D. Type 1 diabetes in young adulthood. Curr Diabetes Rev. 2015; 11(4):239-250.
Mortensen H B, Hougaard P, Swift P, et al. New definition for the partial remission period in children and adolescents with type 1 diabetes. Diabetes Care. 2009; 32:1384-1390.
National Cancer Institute. Common Terminology Criteria for Adverse Events (CTCAE). https://ctep.cancer.gov/protocolDevelopment/electronic_applications/ctc.htm. Updated Mar. 1, 2018. Accessed Sep. 27, 2018.
Ning S, Trislet K, Brown D M et al. Intratumoral radioimmunotherapy of a human colon cancer xenograft using a sustained-release gel. Radiotheray and Oncology. 1996; 39(2):179-189.
Orban T, Bundy B, Becker D J, et al. Co-stimulation modulation with abatacept in patients with recent-onset type 1 diabetes: a randomised, double-blind, placebo-controlled trial. Lancet 2011; 378(9789):412-419.
Palmer J P, Fleming G A, Greenbaum C J, et al. C-peptide is the appropriate outcome measure for type 1 diabetes clinical trials to preserve beta-cell function: report of an ADA workshop, 21-22 Oct. 2001. Diabetes. 2004; 53(1):250-264.
Palmer J P. C-peptide in the natural history of type 1 diabetes. Diabetes Metab Res Rev. 2009; 25(4):325-328.
Rawshani A, Rawshani A, Franzen S, et al. Mortality and Cardiovascular Disease in Type 1 and Type 2 Diabetes. N Engl J Med. 2017; 376(15):1407-1418.
Rawshani A, Sattar N, Franzen S, et al. Excess mortality and cardiovascular disease in young adults with type 1 diabetes in relation to age at onset: a nationwide, register-based cohort study. Lancet. 2018; 392(10146):477-486.
Rigby M R, DiMeglio L A, Rendell M S, et al. Targeting of memory T cells with alefacept in new-onset type ldiabetes (TIDAL study): 12-month results of a randomised, double-blind, placebo-controlled phase 2 trial. Lancet Diabetes Endocrinol. 2013; 1(4):284-294.
Rigby M R, Ehlers M R. Targeted Immune Interventions for Type 1 Diabetes: Not as Easy as it Looks! Curr Opin Endocrinol Diabetes Obes. 2014; 21(4): 271-278.
Rigby M R, Harris K M, Pinckney A, et al. Alefacept provides sustained clinical and immunological effects in new-onset type 1 diabetes patients. J Clin Invest. 2015; 125(8):3285-3296.
Roark C L, Anderson K M, Simon L J, et al. Multiple HLA epitopes contribute to type 1 diabetes susceptibility. Diabetes. 2014; 63(1):323-331.
Rosenzweig M, Rosenthal C A, Torres V M, Vaickus L. Development of a quantitative assay to measure EBV viral load in patients with autoimmune type 1 diabetes and healthy subjects. J Virol Methods. 2010; 164(1-2):111-115.
Sandbom W, Colombel J F, Frankel M, et al. Anti-CD3 antibody visilizumab is not effective in patients with intravenous corticosteroid-refractory ulcerative colitis. Inflammatory bowel disease. 2010; 59(11): 1485-1492.
Saudeck C D, Salem J L, Pitt H A, et al. A Preliminary Trial of the Programmable Implantable Medication System for Insulin Delivery. The New England Journal of Medicine. 1989; 321:574-9.
Seaquist E R, Anderson J, Childs B, et al. Hypoglycemia and diabetes: a report of a workgroup of the American Diabetes Association and the Endocrine Society. Diabetes Care. 2013; 36(5):1384-1395.
Secrest A M, Becker D J, Kelsey S F, LaPorte R E, and Orchard T J. Characterising sudden death and dead-in-bed syndrome in Type 1 diabetes: analysis from 2 childhood-onset Type 1 diabetes registries. Diabet Med. 2011; 28(3): 293-300.
Sefton, M V. Implantable pumps. Crit Rev Biomed Eng. 1987; 14(3):201-40.
Shen W, Taylor B, Laffitte B. Inhibition of DYRK1A and GSK3B induces human 3-cell proliferation. Nature Communications. 2015; 6(8372):1-11.
Sherry N, Hagopian W, Ludvigsson J, et al., Protégé Trial Investigators. Teplizumab for treatment of type 1 diabetes (Protégé study): 1-year results from a randomised, placebo-controlled trial. Lancet. 2011; 378(9790):487-97.
Smolen V F, Ball L. Controlled Drug Bioavailability: Drug Product Design and Performance. Neurotransmitter Receptors. 1984.
Song Y K, Liu D, Maruyama K et al. Antibody Mediated Lung Targeting of Long-Circulating Emulsions. PDA Journal of Pharmaceutical Science and Technology. 1996; 50(6):372-377.
Sorensen J S, Johannesen J, Pociot F, et al. Residual β-cell function 3-6 years after onset of type 1 diabetes reduces risk of severe hypoglycemia in children and adolescents. Diabetes Care. 2013; 36:3454-3459.
Sosenko J M, Skyler J S, Herold K C, et al. The metabolic progression to type 1 diabetes as indicated by serial oral glucose tolerance testing in the Diabetes Prevention Trial-type 1. Diabetes. 2012; 61(6):1331-7.
Sprangers B, Van der Schueren B, Gillard P, et al. Otelixizumab in the treatment of Type 1 diabetes mellitus. Immunotherapy. 2011; 3(11): 1303-1316.
Steck A K, Dong F, Taki I, et al. Early hyperglycemia detected by continuous glucose monitoring in children at risk for type 1 diabetes. Diabetes Care. 2014; 37:2031-2033.
Steffes M W, Sibley S, Jackson M, Thomas W. Beta-cell function and the development of diabetes-related complications in diabetes control and complications trial. Diabetes Care. 2003; 26:832-836.
Streisand R. Young children with type 1 diabetes: challenges, research, and future directions. Curr Diab Rep. 2014; 14(9):520. pp 1-16.
Tahtouh T, Elkins J M, Filippakopoulos P, et al. Selectivity, Cocrystal Structures, and Neuroprotective Properties of Leucettines, a Family of Protein Kinase Inhibitors Derived from the Marine Sponge Alkaloid Leucettamine B. Journal of Medicinal Chemistry. 2012; 55(21):9312-9330.
Trancone A, Bonfanti R, Iafusco D, et al. Evaluating the experience of children with type 1 diabetes and their parents taking part in an artificial pancreas clinical trial over multiple days in a diabetes camp setting. Diabetes Care. 2016; 39:2158-2164.
Tsai C, Robinson P V, Spencer C A, et al. Ultrasensitive Antibody Detection by Agglutination-PCR (ADAP). ACS Central Science. 2016; 2(3):139-147.
Varni J W, Delamater A M, Hood K K, et al. PedsQL 3.2 Diabetes Module for children, adolescents, and young adults: reliability and validity in type 1 diabetes. Diabetes Care. 2018; dc172707.
Verkruyse L A, Storch G A, Devine S M, et al. Once daily ganciclovir as initial pre-emptive therapy delayed until threshold CMV load>or =10000 copies/ml: a safe and effective strategy for allogeneic stem cell transplant patients. Bone Marrow Transplant. 2006; 37(1):51-6.
Vlasakakis G, Napolitano A, Barnard R et al. Target engagement and cellular fate of otelixizumab: a repeat dose escalation study of an anti-CD3E mAb in new-onset type 1 diabetes mellitus patients. British Journal of Clinical Pharmacology. 2019; 85(4):704-714.
Wherrett D K, Chiang J L, Delamater A M, et al. Defining pathways for development of disease-modifying therapies in children with type 1 diabetes: a consensus report. Diabetes Care. 2015; 38(10):1975-85.
Waldron-Lynch F, Henegariu O, Deng S, et al. Teplizumab induces human gut-tropic regulatory cells in humanized mice and patients. Sci Transl Med. 2012; 4(118):118ra12.
Wang P, Alvarez-Perez J C, Felsenfeld D P, Liu H, Sivendran S, Bender A, Kumar A, Sanchez R, Scott D K, Garcia-Oca a A. Stewart A F. A high-throughput chemical screen reveals that harmine-mediated inhibition of DYRK1A increases human pancreatic beta cell replication. Nat Med. 2015 April; 21(4):383-8.
Wang Y J, Golson M L, Schug J, et al. Single-Cell Mass Cytometry Analysis of the Human Endocrine Pancreas. Cell Metabolism. 2016; 24(4):616-626.
Wiczling P, Rosenzweig M, Vaickus L et al. Pharmacokinetics and Pharmacodynamics of a Chimeric/Humanized Anti-CD3 Monoclonal Antibody, Otelixizumab (TRX4), in Subjects With Psoriasis and With Type 1 Diabetes Mellitus. The Journal of Clinical Pharmacology. 2010; 50(5):494-506.
Yu L, Rewers M, Gianani R et al. Antiislet autoantibodies usually develop sequentially rather than simultaneously. The Journal of Clinical Endocrinology & Metabolism. 1996; 81(12):4264-4267.
Ziegler A G and Nepom G T. Prediction and pathogenesis in type 1 diabetes. Immunity. 2010; 32:468-478.

1. A method of treating clinical type 1 diabetes (T1D), comprising administering to a subject in need thereof:
a 12-day to 14-day course of teplizumab at a total dose of about 9000 μg/m² to about 14000 μg/m²; and
an effective amount of a DYRK1A inhibitor,
optionally wherein the total teplizumab dose is about 9000 μg/m² to about 9500 μg/m².

2. (canceled)

3. The method of claim 1, comprising administering to the subject in need thereof a 12-day course of teplizumab, wherein the 12-day course comprises
i) a first dose of 106 μg/m² teplizumab on day 1, a second dose of 425 μg/m² teplizumab on day 2, and one dose of 850 μg/m² teplizumab on each of days 3-12, and wherein the total dose is approximately 9031 μg/m² or
ii) a first dose of 211 μg/m² teplizumab on day 1, a second dose of 423 μg/m² teplizumab on day 2, and one dose of 840 μg/m² teplizumab on each of days 3-12, and wherein the total dose is approximately 9034 μg/m².

4. (canceled)

5. The method of claim 1, comprising administering two 12-day courses of teplizumab, optionally wherein the two courses are administered at an interval of about 6 months.

6. (canceled)

7. The method of claim 5, further comprising administering to the subject in need thereof additional one or more 12-day courses of teplizumab, each additional course at a total dose of about 9000 μg/m² to about 9500 μg/m², optionally wherein each additional course is administered at about 6 months to about 24 months after the preceding course.

8. The method of claim 7, wherein each additional 12-day course of teplizumab comprises
i) a first dose of 106 μg/m² teplizumab on day 1, a second dose of 425 μg/m² teplizumab on day 2, and one dose of 850 μg/m² teplizumab on each of days 3-12, and wherein the total dose of each course is approximately 9031 μg/m², or
ii) a first dose of 211 μg/m² teplizumab on day 1, a second dose of 423 μg/m² teplizumab on day 2, and one dose of 840 μg/m² teplizumab on each of days 3-12, and wherein the total dose of each course is approximately 9034 Ig/m².

9-10. (canceled)

11. The method of claim 1, comprising
determining, optionally by flow cytometry, at baseline and about 3 months after administration, a level of TIGIT+KLRG1+CD8+ T cells with respect to all CD3+ T cells,
monitoring the level of the TIGIT+KLRG1+CD8+CD3+ T-cells, and
administering an additional 12-day course of teplizumab when the level of the TIGIT+KLRG1+CD8+CD3+ T-cells returns to the baseline level.

12-13. (canceled)

14. The method of claim 11, wherein
if the subject has more than about 10% TIGIT+KLRG1+CD8+ T-cells in all CD3+ T cells, subsequent monitoring is annual; and
if the subject has less than about 10% TIGIT+KLRG1+CD8+ T cells in all CD3+ T cells, subsequent monitoring is about every 6 months.

15. (canceled)

16. The method of claim 1, wherein the administering results in reduction of exogenous insulin use, HbA1c levels, hypoglycemic episodes, or combinations thereof by at least 10% as compared to pre-treatment levels, optionally wherein the reduction is over a period of 12 months or more.

17. The method of claim 1, wherein each dose of teplizumab is administered parenterally, optionally subcutaneously or by intravenous infusion.

18-20. (canceled)

21. The method of claim 1, wherein the DYRK1A inhibitor is administered orally, intraperitoneally, subcutaneously or by intravenous infusion.

22. The method of claim 1, wherein teplizumab and the DYRK1A inhibitor are co-administered to the subject in need thereof.

23. The method of claim 1, wherein the subject
i) is about 8 to 17 years old,
ii) is non-diabetic and at risk for T1D,
iii) has a peak C-peptide level of ≥0.2 pmol/mL during a mixed meal tolerance test (MMTT),
iv) has at least 20% of beta-cell function prior to administration of teplizumab and the DYRK1A inhibitor, and/or
v) has a higher mean C-peptide value after treatment with teplizumab and the DYRK1A inhibitor compared with a control receiving placebo.

24-25. (canceled)

26. The method of claim 1, comprising assessing the area under the time-concentration curve (AUC) of C-peptide following a mixed meal tolerance test (MMTT) at 78 weeks.

27-28. (canceled)

29. The method of claim 1, comprising administering a 14 day course of teplizumab, wherein the 14-day course comprises
i) a first dose of about 60 μg/m² teplizumab on day 1, a second dose of about 125 μg/m² teplizumab on day 2, a third dose of about 250 μg/m² teplizumab on day 3, a fourth dose of about 500 μg/m² teplizumab on day 4, and one dose of about 1000 μg/m² teplizumab on each of days 5-14,
ii) a first dose of about 60 μg/m² teplizumab on day 1, a second dose of about 125 μg/m² teplizumab on day 2, a third dose of about 250 μg/m² teplizumab on day 3, a fourth dose of about 500 μg/m² teplizumab on day 4, and one dose of about 1030 μg/m² teplizumab on each of days 5-14;
iii) a first dose of about 100 μg/m² teplizumab on day 1, a second dose of about 425 μg/m² teplizumab on day 2, a third dose of about 850 μg/m² teplizumab on day 3, a fourth dose of about 850 μg/m² teplizumab on day 4, and one dose of about 1000 μg/m² teplizumab on each of days 5-14; or
iv) a first dose of about 65 μg/m² teplizumab on day 1, a second dose of about 125 μg/m² teplizumab on day 2, a third dose of about 250 μg/m² teplizumab on day 3, a fourth dose of about 500 μg/m² teplizumab on day 4, and one dose of about 1070 μg/m² teplizumab on each of days 5-14.

30-37. (canceled)

38. A method of preventing or delaying the onset of type 1 diabetes (T1D), comprising:
administering to a subject in need thereof a 14-day course of teplizumab at a total dose of about 9000 μg/m² to about 14000 μg/m², and
administering to the subject in need thereof an effective amount of a DYRK1A inhibitor.

39. The method of claim 38, wherein teplizumab is administered subcutaneously (SC) or intravenously (IV).

40. The method of claim 38, wherein the 14 day course comprises
i) a first IV dose of about 60 μg/m² teplizumab on day 1, a second IV dose of about 125 μg/m² teplizumab on day 2, a third IV dose of about 250 μg/m² teplizumab on day 3, a fourth IV dose of about 500 μg/m² teplizumab on day 4, and one IV dose of about 1,000 g/m² teplizumab on each of days 5-14;
ii) a first IV dose of about 60 μg/m² teplizumab on day 1, a second IV dose of about 125 ag/m² teplizumab on day 2, a third IV dose of about 250 μg/m² teplizumab on day 3, a fourth IV dose of about 500 μg/m² teplizumab on day 4, and one IV dose of about 1,030 μg/m² teplizumab on each of days 5-14;
iii) a first IV dose of about 100 μg/m² teplizumab on day 1, a second IV dose of about 425 μg/m² teplizumab on day 2, a third IV dose of about 850 μg/m² teplizumab on day 3, a fourth IV dose of about 850 μg/m² teplizumab on day 4, and one IV dose of about 1,000 μg/m² teplizumab on each of days 5-14; or
iv) a first IV dose of about 65 μg/m² teplizumab on day 1, a second IV dose of about 125 ag/m² teplizumab on day 2, a third IV dose of about 250 μg/m² teplizumab on day 3, a fourth IV dose of about 500 μg/m² teplizumab on day 4, and one IV dose of about 1,070 μg/m² teplizumab on each of days 5-14.

41-43. (canceled)

44. A method of treating type 1 diabetes (T1D), comprising:
administering intravenously or subcutaneously to a subject in need thereof a 12-day course of teplizumab at a total dose of about 9000 μg/m² to about 14000 μg/m²; and
administering to the subject in need thereof an effective amount of a DYRK1A inhibitor.

45-47. (canceled)

48. The method of claim 1, wherein the DYRK1A inhibitor is selected from the group consisting of harmine, INDY, DANDY, FINDY, leucettine, GNF4877, 5-iodotubericidin, CC-401, epigallocatechin, quinalizarine, peltogynoids Acanilol A and B, benzocoumarins (dNBC), indolocarbazoles (e.g., staurosporine and rebeccamycin), pyrazolidinediones, amino-quinazolines, meriolins, pyridine and pyrazines, chromenoidoles, 11H-indolo[3,2-c]quinoline-6-carboxylic acids, and thiazolo[5,4-f]quinazolines (EHT 5372), and derivatives and pharmaceutically acceptable salts thereof.
